(19) United States
(12) Patent Application Publication
(10) Pub. No.: US 2017/0275298 A1
(43) Pub. Date: Sep. 28, 2017

(54) INHIBITORS OF THE RENAL OUTER MEDULLARY POTASSIUM CHANNEL

(71) Applicant: Merck Sharp & Dohme Corp., Rahway, NJ (US)

(72) Inventors: Alexander Pasternak, Princeton, NJ (US); Shuzhi Dong, Plainsboro, NJ (US); Xin Gu, Scotch Plains, NJ (US); Jinlong Jiang, Scotch Plains, NJ (US); Zhi-Cai Shi, Monmouth Junction, NJ (US); Shawn P. Walsh, Bridgewater, NJ (US); Zhicai Wu, Montvale, NJ (US); Yang Yu, Clark, NJ (US); Ronald Ferguson, II, Scotch Plains, NJ (US); Zhiqiang Guo, Morganville, NJ (US); Jessica Frie, Harleysville, PA (US); Takao Suzuki, Shanghai (CN); Timothy A. Blizzard, Princeton, NJ (US); Qinghong Fu, Plainsboro, NJ (US); Kelsey F. Vangelder, Philadelphi, PA (US)

(21) Appl. No.: 15/505,254

(22) PCT Filed: Oct. 26, 2015
(86) PCT No.: PCT/US15/57283

§ 371 Date: Feb. 21, 2017

(30) Foreign Application Priority Data
Oct. 30, 2014 (CN) PCT/CN2014/089930

(51) Int. Cl. C07D 498/10; A61K 31/435; A61K 31/501; A61K 31/506; A61K 31/444; A61K 31/5386; A61K 31/497; A61K 31/41; A61K 31/4184; A61K 31/4178; A61K 31/4422; A61K 31/55; A61K 31/401; A61K 31/675; A61K 31/551; A61K 31/4965; A61K 31/585; C07D 471/10

(52) U.S. Cl. CPC C07D 498/10; C07D 471/10; A61K 31/435; A61K 31/501; A61K 31/506; A61K 31/444; A61K 31/5386; A61K 31/497; A61K 31/41; A61K 31/4184; A61K 31/4178; A61K 31/4422; A61K 31/55; A61K 31/401; A61K 31/675; A61K 31/551; A61K 31/4965; A61K 31/585

(57) ABSTRACT

The present invention provides compounds of Formula I and the pharmaceutically acceptable salts thereof, which are inhibitors of the ROMK (Kir1.1) channel. The compounds may be used as diuretic and/or natriuretic agents and for the therapy and prophylaxis of medical conditions including cardiovascular diseases such as hypertension, heart failure and chronic kidney disease and conditions associated with excessive salt and water retention.

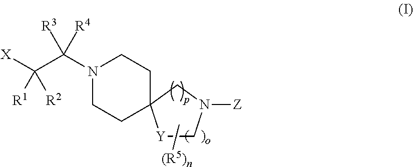

### BACKGROUND OF THE INVENTION

The Renal Outer Medullary Postassium (ROMK) channel Kir1.1) (see e.g., Ho, K., et al., Cloning and expression of an inwardly rectifying ATP-regulated potassium channel, Nature, 1993, 362(6415): p. 31-8.1, 2; and Shuck, M. E., et al., Cloning and characterization of multiple forms of the human kidney ROM-K potassium channel, J Biol Chem, 1994, 269(39): p. 24261-70) is a member of the inward rectifier family of potassium channels expressed in two regions of the kidney: thick ascending loop of Henle (TALH) and cortical collecting duct (CCD) (see Hebert, S. C., et al., Molecular diversity and regulation of renal potassium channels, Physiol Rev, 2005, 85(1): p. 319-713). At the TALH, ROMK participates in potassium recycling across the luminal membrane which is critical for the function of the Na⁺/K⁺/2Cl⁻ co-transporter, the rate-determining step for salt reuptake in this part of the nephron. At the CCD, ROMK provides a pathway for potassium secretion that is tightly coupled to sodium uptake through the amiloride-sensitive sodium channel (see Reinalter, S. C., et al., Pharmacotyping of hypokalaemic salt-losing tubular disorders, Acta Physiol Scand, 2004, 181(4): p. 513-21; and Wang, W., Renal potassium channels: recent developments, Curr Opin Nephrol Hypertens, 2004, 13(5): p. 549-55). Selective inhibitors of the ROMK channel (also referred to herein as inhibitors of ROMK or ROMK inhibitors) are expected to represent novel diuretics for the treatment of hypertension and other conditions where treatment with a diuretic would be beneficial with potentially reduced liabilities (i.e., hypo- or hyperkalemia, new onset of diabetes, dyslipidemia) over the currently used clinical agents (see Lifton, R. P., A. G. Gharavi, and D. S. Geller, Molecular mechanisms of human hypertension, Cell, 2001, 104(4): p. 545-56). Human genetics (Ji, W., et al., Rare independent mutations in renal salt handling genes contribute to blood pressure variation, Nat Genet, 2008, 40(5): p. 592-9; and Tobin, M. D., et al., Common variants in genes underlying monogenic hypertension and hypotension and blood pressure in the general population, Hypertension, 2008, 51(6): p. 1658-64) and genetic ablation of ROMK in rodents (see Lorenz, J. N., et al., Impaired renal NaCl absorption in mice lacking the ROMK potassium channel, a model for type II Bartter's syndrome, J Biol Chem, 2002, 277(40): p. 37871-80 and Lu, M., et al., Absence of small conductance K+ channel (SK) activity in apical membranes of thick ascending limb and cortical collecting duct in ROMK (Bartter's) knockout mice, J Biol Chem, 2002, 277(40): p. 37881-7) support these expectations. To our knowledge, the first publicly disclosed small molecule selective inhibitors of ROMK, including VU590, were reported from work done at Vanderbilt University as described in Lewis, L. M., et al., High-Throughput Screening Reveals a Small-Molecule Inhibitor of the Renal Outer Medullary Potassium Channel and Kir7.1, Mol Pharmacol, 2009, 76(5): p. 1094-1103. The compound VU591 was later reported in Bhave, G. et al., Development of a Selective Small-Molecule Inhibitor of Kir1.1, the Renal Outer Medullary Potassium Channel, Mol Pharmacol, 2011, 79(1), p. 42-50, the text of which states that “ROMK (Kir1.1), is a putative drug target for a novel class of loop diuretics that would lower blood pressure without causing hypokalemia.”

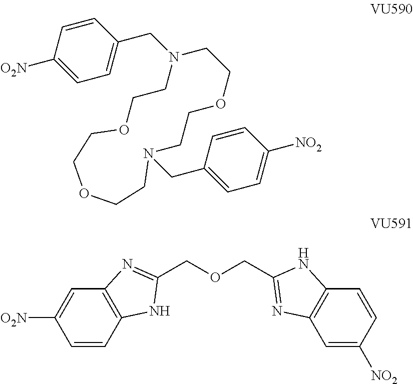 [SMILES: O=[N+]([O-])c1ccc(CN2CCOCCOCCN(Cc3ccc([N+](=O)[O-])cc3)CCOCC2)cc1 ; O=[N+]([O-])c1ccc2[nH]c(COCc3nc4cc([N+](=O)[O-])ccc4[nH]3)nc2c1]

Patent application publication number WO2010/129379, published Nov. 11, 2010 having common representative Merck Sharp & Dohme Corp., (also published as US2010/0286123 on same date), describes ROMK inhibitors having the generic formula:

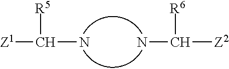

and, e.g., an embodiment

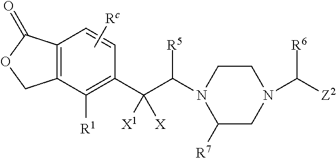

wherein R⁵ and R⁶ are independently —H, —C₁₋₆ alkyl, —C₃₋₆ cycloalkyl, —CF₃, —CHF₂, —CH₂F or —CH₂OH; X is —H, —OH, —OC₁₋₃alkyl, —F, oxo, NH₂ or —CH₃; and X¹ is —H or —CH₃.

Patent application publication number WO2012/058134, published May 3, 2012, having common representative Merck Sharp & Dohme Corp., describes ROMK inhibitors having the generic formula:

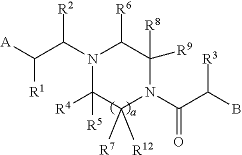

wherein A and B are mono and/or bicyclic aromatic groups; R² is —H,
—C₁₋₆ alkyl, —C₃₋₆ cycloalkyl, CF₃, —CH₂OH, or —CO₂R, or R² can be joined to R¹ or R^{10a} to form a ring; R³ is —H, —C₁₋₆ alkyl, —C₃₋₆ cycloalkyl, —OH, —F, —OC₁₋₃ alkyl, or —CH₂OH, or R³ can be joined to R^{10a} to form a ring.

Patent application publication number WO2012/058116, published May 3, 2012, having common representative Merck Sharp & Dohme Corp., describes ROMK inhibitors having the generic formula:

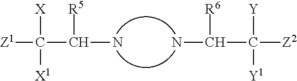

and, e.g., an embodiment

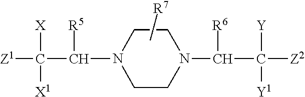

wherein R⁵ and R⁶ are independently —H, —C₁₋₆ alkyl or —C(O)OC₁₋₃alkyl; and X, X¹, Y and Y¹ are independently —H or —C₁₋₆alkyl; or Y¹ can be joined together with Z² to form a fused ring system. Additional published patent applications to Merck Sharp and Dohme Corp., which describe ROMK inhibitors, include: WO2013/028474; WO2013/039802; WO2013/062892; WO2013/066714; WO2013/066717; WO2013/066718; and WO2013/090271.

However, continuing discovery of selective small molecule inhibitors of ROMK is still needed for the development of new treatments for hypertension, heart failure, edematous states and related disorders. The compounds of Formula I and salts thereof of this invention are selective inhibitors of the ROMK channel and could be used for the treatment of hypertension, heart failure and other conditions where treatment with a diuretic or natriuretic would be beneficial.

### SUMMARY OF THE INVENTION

The present invention provides for compounds of the Formula of the formula

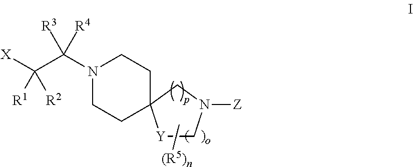

or a pharmaceutically acceptable salt thereof,

wherein:

X is

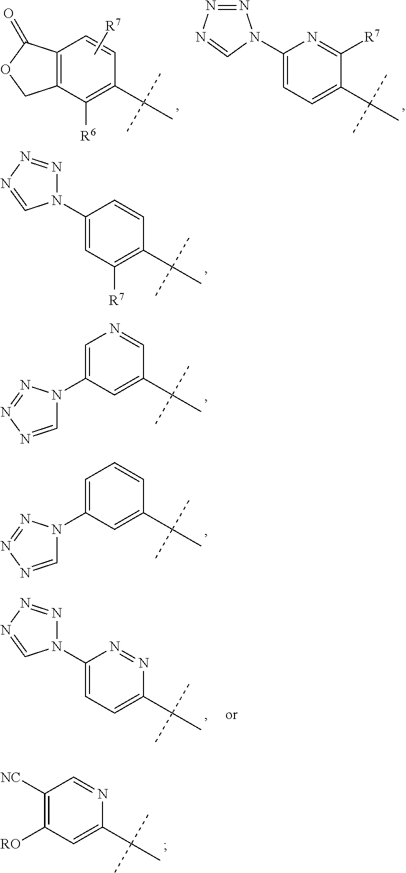

Y is —O—, —NH— or a bond;

Z is

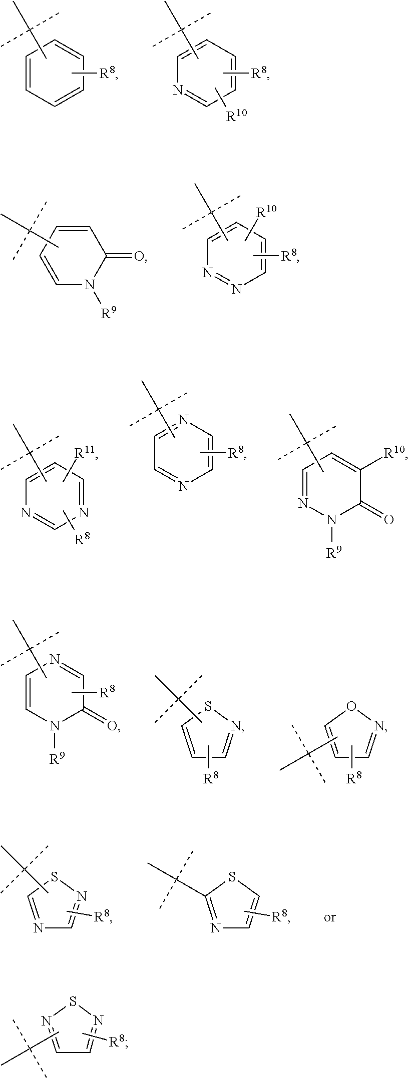

R is independently H, alkyl (e.g., methyl or ethyl) or haloalkyl (e.g., —CHF₂ or —CF₃);

R¹ is H, D or —OH;

R² is H or D;

R³ is H or D;

R⁴ is H, D, or alkyl (e.g., methyl or ethyl);

R⁵ is independently oxo or alkyl optionally substituted by 1-5 fluorine atoms;

R⁶ is H or alkyl (e.g., methyl or ethyl);

R⁷ is H or alkyl (e.g., methyl or ethyl);

R⁸ is H; halo; alkyl (e.g., methyl or ethyl) optionally substituted by —OR, —C(O)OR¹², —OC(O)—R¹², or 1-5 halogen atoms (e.g., —CHF₂ or —CF₃); —OR (e.g., methoxy or ethoxy); phenyl; —C(O)OR¹³; —N(R¹⁴)(R¹⁵); furanyl; or —OCD₃;

R⁹ is H, alkyl (e.g., methyl or ethyl) optionally substituted by 1-5 halogen atoms (e.g., —CHF₂ or —CF₃) or cycloalkyl e.g. (cyclopropyl);

R¹⁰ is H, halo, or alkyl (e.g., methyl or ethyl) optionally substituted by 1-5 halogen atoms (e.g., —CHF₂ or —CF₃);

R¹¹ is H, alkyl (e.g., methyl or ethyl) optionally substituted by 1-5 halogen atoms (e.g., methyl or ethyl) or —OR (e.g., methoxy or ethoxy);

R¹² is H or alkyl (e.g., methyl or ethyl);

R¹³ is H or alkyl (e.g., methyl or ethyl);

R¹⁴ is H, alkyl (e.g., methyl or ethyl);

R¹⁵ is H or alkyl (e.g., methyl or ethyl);

n is 0, 1 or 2;

o is 1, 2 or 3; and

p is 1 or 2.

The compound of Formula I are inhibitors of the ROMK (Kir1.1) channel. As a result, the compounds of Formula I could be used in methods of treatment, inhibition or amelioration of one or more disease states that could benefit from inhibition of ROMK. The compounds of this invention could be used in methods of treatment which comprise administering a therapeutically or prophylactically effective amount of a compound of Formula I to a patient in need of a diuretic and/or natriuretic agent. Therefore, the compounds of Formula I could be valuable pharmaceutically active compounds for the therapy, prophylaxis or both of medical conditions, including, but not limited to, cardiovascular diseases such as hypertension and heart failure as well as chronic kidney disease, and conditions associated with excessive salt and water retention. The compounds of this invention could further be used in combination with other therapeutically effective agents, including but not limited to, other drugs which are useful for the treatment of hypertension, heart failure and conditions associated with excessive salt and water retention. The invention furthermore relates to processes for preparing compounds of Formula I, and pharmaceutical compositions which comprise compounds of Formula I. These and other aspects of the invention will be evident from the description contained herein.

### DETAILED DESCRIPTION OF THE INVENTION

An embodiment of this invention is compounds of Formula I or pharmaceutically acceptable salts thereof.

Another embodiment of this invention is a compound of Formula I having the structural formula

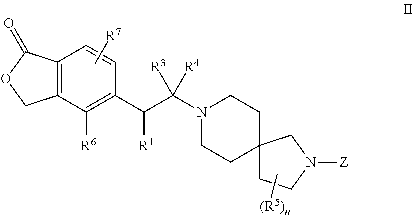

or a pharmaceutically acceptable salt thereof.

wherein R¹, R³, R⁴, R⁵, R⁶, R⁷, Z, and n are as defined in Formula I.

Another embodiment of this invention is a compound of Formula II having the structural formula:

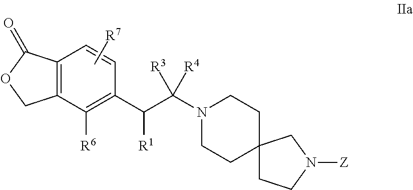

wherein:

Z is

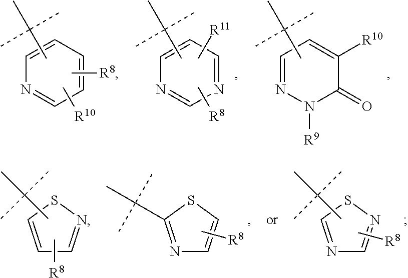

and

R¹, R³, R⁴, R⁶, R⁷, R⁸, R⁹, R¹⁰, and R¹¹ are as defined in Formula I.

Another embodiment of this invention is a compound of Formula II having the structural formula:

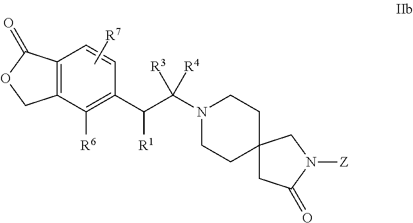

wherein

Z is

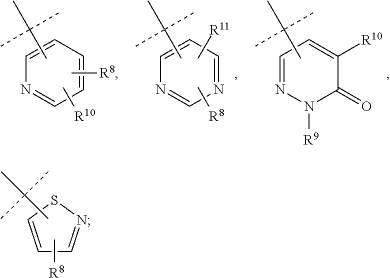

and

R¹, R³, R⁴, R⁶, R⁷, R⁸, R⁹, R¹⁰, and R¹¹ are as defined in Formula I.

Another embodiment of this invention is compound of Formula II having the structural formula:

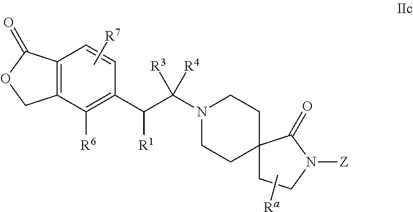

wherein:

R^{a} is H or alkyl;

Z is

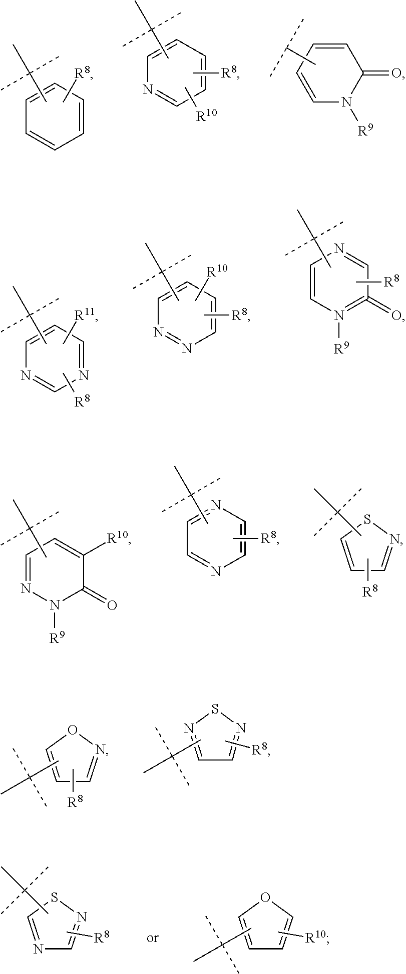

and

R¹, R³, R⁴, R⁶, R⁷, R⁸, R⁹, R¹⁰, and R¹¹ are as defined in Formula I.

Another embodiment of this invention is compound of Formula I having the structural formula:

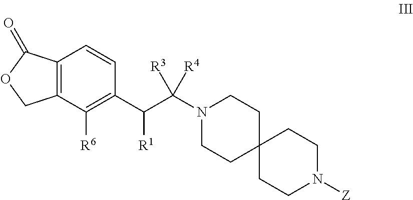

or a pharmaceutically acceptable salt thereof

wherein:

Z is

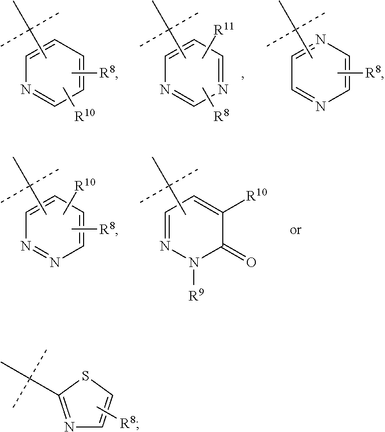

and

R¹, R³, R⁴, R⁶, R⁸, R⁹, R¹⁰, and R¹¹ are as defined in Formula I.

Another embodiment of this invention is compound of Formula I having the structural formula:

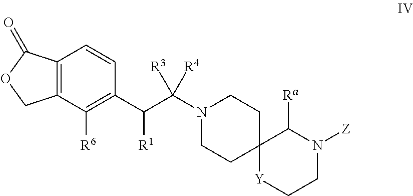

or a pharmaceutically acceptable salt thereof

wherein:

R^{a} is H or oxo;

Y is —O— or —NH—;

Z is

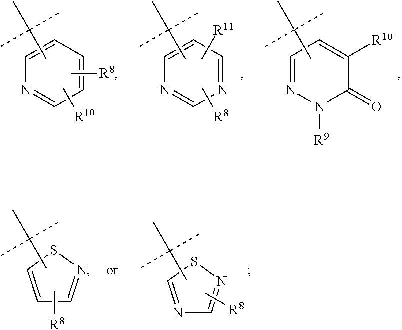

and

R¹, R³, R⁴, R⁶, R⁸, R⁹, R¹⁰, and R¹¹ are as defined in Formula I.

Another embodiment of this invention is a compound of Formula I having the structural formula:

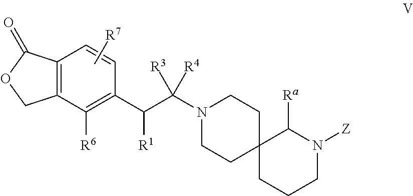

or a pharmaceutically acceptable salt thereof

wherein:

R^{a} is H or oxo;

Y is —O— or —NH—;

Z is

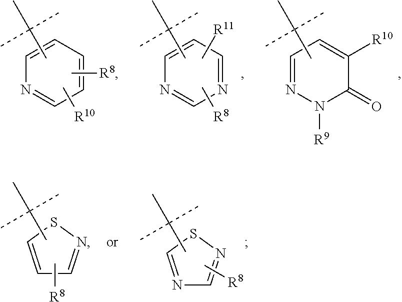

and

R¹, R³, R⁴, R⁶, R⁷, R⁸, R⁹, R¹⁰, and R¹¹ are as defined in Formula I.

Another embodiment of this invention is a compound of Formula I having the structural formula:

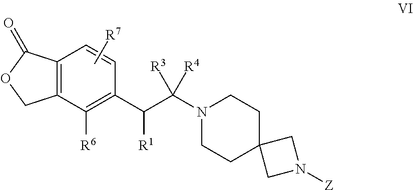

or a pharmaceutically acceptable salt thereof,

wherein:

Z is

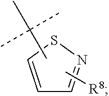

and

R¹, R³, R⁴, R⁶, R⁷, and R⁸ are as defined in Formula I.

Another embodiment of this invention is a compound of Formula I having the structural formula:

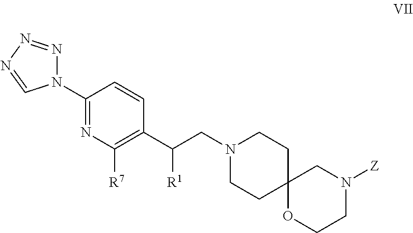

or a pharmaceutically acceptable salt thereof
wherein:

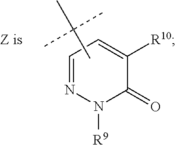

and

R¹, R⁷, R⁹ and R¹⁰ are as defined in Formula I.

Another embodiment of this invention is a compound of Formula I having the structural formula:

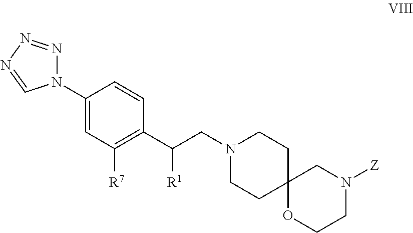

or a pharmaceutically acceptable salt thereof,
wherein:

Z is

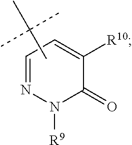

and

R¹, R⁷, R⁹ and R¹⁰ are as defined in Formula I.

Another embodiment of this invention is a compound of Formula I having the structural formula:

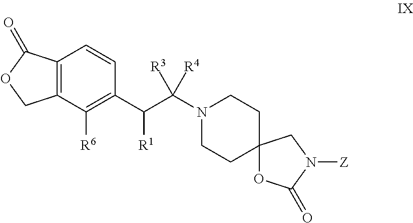

or a pharmaceutically acceptable salt thereof

wherein:

Z is

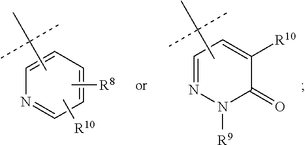

and

R¹, R³, R⁴, R⁶, R⁸, R⁹, and R¹⁰ are as defined in Formula I.

Another embodiment of this invention is a compound of Formula I having the structural formula:

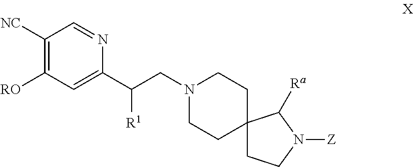

or a pharmaceutically acceptable salt thereof,

wherein:

R^{a} is H or oxo; and

Z is

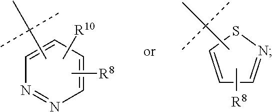

and

R, R¹, R⁸, and R¹⁰ are as defined in Formula I.

Another embodiment of this invention is a compound of Formula I having the structural formula:

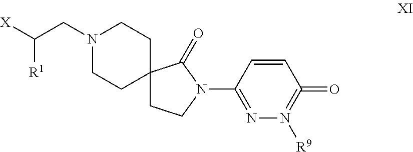

or a pharmaceutically acceptable salt thereof

wherein:

X is

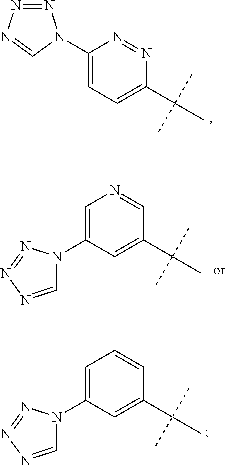

and

R¹ and R⁹ are as defined in Formula I.

Another embodiment of this invention is a compound of Formula I having the structural formula:

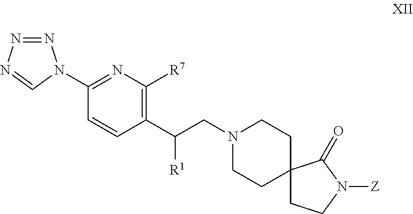

or a pharmaceutically acceptable salt thereof,

wherein:

Z is

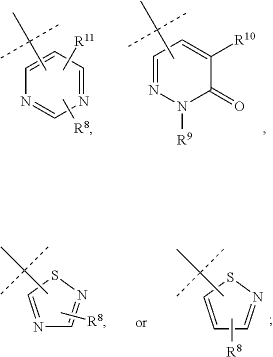

and

R¹, R⁷, R⁸, R⁹, R¹⁰, and R¹¹ are as defined in Formula I.

Another embodiment of this invention is a compound of Formula I having the structural formula:

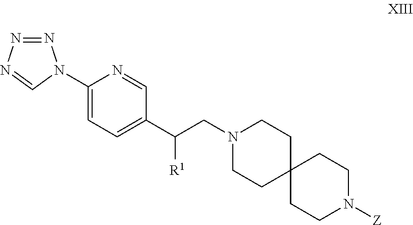

or a pharmaceutically acceptable salt thereof

wherein:

R¹ is H or —OH

Z is

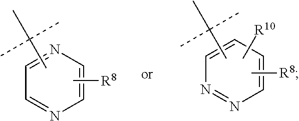

and

R⁸ is as defined in Formula I.

Another embodiment of the present invention is compounds of Formulae I, II, IIa, IIb, IIc, V, and VI above or their pharmaceutically acceptable salts wherein R¹ is —OH, R³ is H and R⁴ is H, R⁶ is methyl and R⁷ is H.

Another embodiment of the present invention is compounds of Formulae I, X, XI, or XII or their pharmaceutically acceptable salts wherein R¹ is H.

Another embodiment of the present invention is compounds of Formula I, II, IIa, IIb, IIc, III, IV, V. VII, VIII, IX and XII wherein Z is:

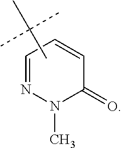

Another embodiment is a compound which is:
(R)-8-(2-hydroxy-2-(4-methyl-1-oxo-1,3-dihydroisobenzofuran-5-yl)ethyl)-2-(isothiazol-5-yl)-2,8-diazaspiro[4.5]decan-1-one;
(R)-8-(2-hydroxy-2-(4-methyl-1-oxo-1,3-dihydroisobenzofuran-5-yl)ethyl)-2-(pyridin-4-yl)-2,8-diazaspiro[4.5]decan-1-one;
(R)-8-(2-hydroxy-2-(4-methyl-1-oxo-1,3-dihydroisobenzofuran-5-yl)ethyl)-2-(3-methylisothiazol-5-yl)-2,8-diazaspiro[4.5]decan-1-one
(R)-8-(2-hydroxy-2-(4-methyl-1-oxo-1,3-dihydroisobenzofuran-5-yl)ethyl)-2-(3-methyl-1,2,4-thiadiazol-5-yl)-2,8-diazaspiro[4.5]decan-1-one
(R)-8-(2-hydroxy-2-(4-methyl-1-oxo-1,3-dihydroisobenzofuran-5-yl)ethyl)-2-(isothiazol-4-yl)-2,8-diazaspiro[4.5]decan-1-one;
(R)-8-(2-hydroxy-2-(4-methyl-1-oxo-1,3-dihydroisobenzofuran-5-yl)ethyl)-2-(2-methoxypyridin-4-yl)-2,8-diazaspiro[4.5]decan-1-one;
(R)-8-(2-hydroxy-2-(4-methyl-1-oxo-1,3-dihydroisobenzofuran-5-yl)ethyl)-2-(3-methoxy-1,2,4-thiadiazol-5-yl)-2,8-diazaspiro[4.5]decan-1-one;
(R)-8-(2-hydroxy-2-(4-methyl-1-oxo-1,3-dihydroisobenzofuran-5-yl)ethyl)-2-(pyridin-4-yl)-2,8-diazaspiro[4.5]decan-3-one;
(R)-8-(2-hydroxy-2-(4-methyl-1-oxo-1,3-dihydroisobenzofuran-5-yl)ethyl)-2-(2-methoxypyrimidin-4-yl)-2,8-diazaspiro[4.5]decan-1-one;
(R)-8-(2-hydroxy-2-(4-methyl-1-oxo-1,3-dihydroisobenzofuran-5-yl)ethyl)-2-(2-methoxypyrimidin-4-yl)-2,8-diazaspiro[4.5]decan-1-one (methoxy-d₃);
(R)-8-(2-hydroxy-2-(4-methyl-1-oxo-1,3-dihydroisobenzofuran-5-yl)ethyl)-2-(2-(methylamino)pyrimidin-4-yl)-2,8-diazaspiro[4.5]decan-1-one;
(R)-8-(2-hydroxy-2-(4-methyl-1-oxo-1,3-dihydroisobenzofuran-5-yl)ethyl)-2-(1-methyl-6-oxo-1,6-dihydropyridazin-3-yl)-2,8-diazaspiro[4.5]decan-1-one;
(R)-8-(2-hydroxy-2-(4-methyl-1-oxo-1,3-dihydroisobenzofuran-5-yl)ethyl-d₃)-2-(1-methyl-6-oxo-1,6-dihydropyridazin-3-yl)-2,8-diazaspiro[4.5]decan-1-one;
(R)-8-((1R,2S)-1-hydroxy-1-(4-methyl-1-oxo-1,3-dihydroisobenzofuran-5-yl)propan-2-yl)-2-(1-methyl-6-oxo-1,6-dihydropyridazin-3-yl)-2,8-diazaspiro[4.5]decan-1-one;
(R)-8-((1S,2S)-1-hydroxy-1-(4-methyl-1-oxo-1,3-dihydroisobenzofuran-5-yl)propan-2-yl)-2-(1-methyl-6-oxo-1,6-dihydropyridazin-3-yl)-2,8-diazaspiro[4.5]decan-1-one; or
(R)-9-(2-hydroxy-2-(4-methyl-1-oxo-1,3-dihydroisobenzofuran-5-yl)ethyl)-4-(3-methoxy-1,2,4-thiadiazol-5-yl)-1-oxa-4,9-diazaspiro[5.5]undecan-5-one
   or a pharmaceutically acceptable salt thereof.

All structural formulae, embodiments and classes thereof described herein include the pharmaceutically acceptable salts of the compounds defined therein.

As used herein except if noted otherwise, “D refers to deuterium and when it is used with respect to a specific position in a formula or structure, it means that the dueterium in this position is enriched with deuterium that is above the level of naturally distribution of deuterium.

“Alkyl” is intended to include both branched- and straight-chain saturated aliphatic hydrocarbon groups having, e.g., 1-12, 1-6 or 1-4 carbon atoms. Commonly used abbreviations for alkyl groups are used throughout the specification. For example the term “C₁₋₆ alkyl” (or “C₁-C₆ alkyl”), means linear or branched chain alkyl groups, including all isomers, having the specified number of carbon atoms and includes all of the hexyl and pentyl isomers as well as n-, iso-, sec- and tert-butyl (butyl, s-butyl, i-butyl, t-butyl; Bu=butyl), n- and i-propyl (Pr=propyl), ethyl (Et) and methyl (Me).

“Halogen” means a fluorine, chlorine, bromine or iodine atom. “Halo” means —F, —Cl, —Br, or —I. A non-limiting example includes fluorine or fluoro.

“Haloalkyl” means a halo-alkyl group in which the halo and alkyl groups are as previously defined. The bond to the parent moiety is through the alkyl group. Non-limiting examples include —CH₂CF₃ and —CF₃.

“Cycloalkyl” is a cyclized alkyl ring having 3-12 or 3-6 carbon atoms. Examples of cycloalkyl include cyclopropyl, cyclobutyl, cyclopentyl and cyclohexyl.

“Oxo” is a “C=(0)” functional group, that is a carbonyl group.

Unless expressly depicted or described otherwise, variables depicted in a structural formula with a “floating” bond, such as R⁵ and R⁸, are permitted on any available carbon atom in the ring to which the variable is attached.

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Where a substituent or variable has multiple definitions, it is understood that the substituent or variable is defined as being selected from the group consisting of the indicated definitions.

The compounds of Formula I may have one or more chiral (asymmetric) centers. The present invention encompasses all stereoisomeric forms of the compounds of Formula I. Centers of asymmetry that are present in the compounds of Formula I can all independently of one another have (R) or (S) configuration. When bonds to a chiral carbon are depicted as straight lines in the structural Formulas of the invention, or when a compound name is recited without an (R) or (S) chiral designation for a chiral carbon, it is understood that both the (R) and (S) configurations of each such chiral carbon, and hence each enantiomer or diastereomer and mixtures thereof, are embraced within the Formula or by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis. Where compounds of this invention are capable of tautomerization, all individual tautomers as well as mixtures thereof are included in the scope of this invention. The present invention includes all such isomers, as well as salts, solvates (which includes hydrates) and solvated salts of such racemates, enantiomers, diastereomers and tautomers and mixtures thereof.

Reference to the compounds of Formula I herein encompasses the compounds of Formulae I-XIII and all embodiments and classes thereof. Reference to the compounds of this invention as those of a specific formula or embodiment, e.g., Formulae I-XIII or embodiments thereof, or any other generic structural formula or specific compound described or claimed herein, is intended to encompass the specific compound or compounds falling within the scope of the Formula or embodiment, including salts thereof, particularly pharmaceutically acceptable salts, solvates (including hydrates) of such compounds and solvated salt forms thereof, where such forms are possible, unless specified otherwise.

In the compounds of Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of Formula I. For example, different isotopic forms of hydrogen (H) include protium (1H) and deuterium (2H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within Formula I can be prepared without undue experimentation by conventional techniques well known to those skilled in the art or by processes analogous to those described in the Schemes and Examples herein using appropriate isotopically-enriched reagents and/or intermediates.

When the compounds of Formula I contain one or more acidic or basic groups the invention also includes the corresponding pharmaceutically acceptable salts. Thus, the compounds of Formula I which contain acidic groups can be used according to the invention as, for example but not limited to, alkali metal salts, alkaline earth metal salts or as ammonium salts. Examples of such salts include but are not limited to sodium salts, potassium salts, calcium salts, magnesium salts or salts with ammonia or organic amines such as, for example, ethylamine, ethanolamine, triethanolamine or amino acids. Compounds of Formula I which contain one or more basic groups, i.e. groups which can be protonated, can be used according to the invention in the form of their acid addition salts with inorganic or organic acids as, for example but not limited to, salts with hydrogen chloride, hydrogen bromide, phosphoric acid, sulfuric acid, nitric acid, benzenesulfonic acid, methanesulfonic acid, p-toluenesulfonic acid, naphthalenedisulfonic acids, oxalic acid, acetic acid, trifluoroacetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, etc. If the compounds of Formula I simultaneously contain acidic and basic groups in the molecule the invention also includes, in addition to the salt forms mentioned, inner salts or betaines (zwitterions). Salts can be obtained from the compounds of Formula I by customary methods which are known to the person skilled in the art, for example by combination with an organic or inorganic acid or base in a solvent or dispersant, or by anion exchange or cation exchange from other salts. The present invention also includes all salts of the compounds of Formula I which, owing to low physiological compatibility, are not directly suitable for use in pharmaceuticals but which can be used, for example, as intermediates for chemical reactions or for the preparation of pharmaceutically acceptable salts.

Furthermore, compounds of the present invention may exist in amorphous form and/or one or more crystalline forms, and as such all amorphous and crystalline forms and mixtures thereof of the compounds of Formula I are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms.

Any pharmaceutically acceptable pro-drug modification of a compound of this invention which results in conversion in vivo to a compound within the scope of this invention is also within the scope of this invention. For example, esters can optionally be made by esterification of an available carboxylic acid group or by formation of an ester on an available hydroxy group in a compound. Similarly, labile amides can be made. Pharmaceutically acceptable esters or amides of the compounds of this invention may be prepared to act as pro-drugs which can be hydrolyzed back to an acid (or —COO— depending on the pH of the fluid or tissue where conversion takes place) or hydroxy form particularly in vivo and as such are encompassed within the scope of this invention. Examples of pharmaceutically acceptable pro-drug modifications include, but are not limited to, —C₁₋₆alkyl esters and —C₁₋₆alkyl substituted with phenyl esters.

Accordingly, the compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof, pro-drug forms thereof, and salts of pro-drug forms thereof, where such forms are possible unless specified otherwise.

The compounds of Formula I according to the invention are inhibitors of ROMK, and therefore could be used as diuretic and/or natriuretic agents. ROMK inhibitors may be used to help to increase urination and increase urine volume and also to prevent or reduce reabsorption of sodium in the kidneys leading to increased excretion of sodium and water. Therefore, the compounds could be used for treatment or prophylaxis or both of disorders that benefit from increased excretion of water and sodium from the body. Accordingly, the compounds of this invention could be used in a method for inhibiting ROMK comprising administering a compound of Formula I in a ROMK-inhibitory effective amount to a patient in need thereof. This also encompasses the use of the compounds for inhibiting ROMK in a patient comprising administering a compound of claim 1 in a therapeutically effective amount to a patient in need of diueresis, natriuresis or both. The inhibition of ROMK by the compounds of Formula I can be examined, for example, in the Thallium Flux Assay described below. Moreover, this invention also relates to the use of the compounds of Formula I or salts thereof to validate in vitro assays, for example but not limited to the Thallium Flux Assay described herein.

The compounds of this invention could be used in a method for causing diuresis, natriuresis or both, comprising administering a compound of Formula I in a therapeutically effective amount to a patient in need thereof. Therefore, the compounds of Formula I of this invention could be used in methods for treatment of, prevention of or reduction of risk for developing medical conditions that benefit from increased excretion of water and sodium, such as but not limited to one or more of hypertension, such as essential hypertension (also known as primary or idiopathic hypertension) which is a form of hypertension for which no cause can be found, heart failure (which includes both acute heart failure and chronic heart failure, the latter also known as congestive heart failure) and/or other conditions associated with excessive salt and water retention. The compounds could also be used to treat hypertension which is associated with any of several primary diseases, such as renal, pulmonary, endocrine, and vascular diseases, including treatment of patients with medical conditions such as heart failure and/or chronic kidney disease. Furthermore, the compounds of Formula I could be used in methods for treatment of, prevention of or reduction of risk for developing one or more disorders such as pulmonary hypertension, particularly pulmonary arterial hypertension (PAH), cardiovascular disease, edematous states, diabetes mellitus, diabetes insipidus, post-operative volume overload, endothelial dysfunction, diastolic dysfunction, systolic dysfunction, stable and unstable angina pectoris, thromboses, restenosis, myocardial infarction, stroke, cardiac insufficiency, pulmonary hypertonia, atherosclerosis, hepatic cirrhosis, ascitis, pre-eclampsia, cerebral edema, nephropathy, glomerulonephritis, nephrotic syndrome, acute kidney insufficiency, chronic kidney insufficiency (also referred to as chronic kidney disease, or more generally as renal impairment), acute tubular necrosis, hypercalcemia, idiopathic edema, Dent's disease, Meniere's disease, glaucoma, benign intracranial hypertension, and other conditions for which a diuretic or natriuretic or both would have therapeutic or prophylactic benefit. The compounds of the invention may be administered to a patient having, or at risk of having, one or more conditions for which a diuretic or natriuretic or both would have therapeutic or prophylactic benefit such as those described herein.

The compounds of Formula I may potentially have reduced liabilities (for example, hypo- or hyperkalemia, new onset of diabetes, dyslipidemia, etc.) over currently used clinical agents. Also the compounds may have reduced risk for diuretic tolerance, which can be a problem with long-term use of loop diuretics.

In general, compounds that are ROMK inhibitors can be identified as those compounds which, when tested, have an IC₅₀ of 5 μM or less, preferably 1 μM or less, and more preferably 0.25 μM or less, in the Thallium Flux Assay, described in more detail further below.

The dosage amount of the compound to be administered depends on the individual case and is, as is customary, to be adapted to the individual circumstances to achieve an optimum effect. Thus, it depends on the nature and the severity of the disorder to be treated, and also on the sex, age, weight and individual responsiveness of the human or animal to be treated, on the efficacy and duration of action of the compounds used, on whether the therapy is acute or chronic or prophylactic, or on whether other active compounds are administered in addition to compounds of Formula I. A consideration of these factors is well within the purview of the ordinarily skilled clinician for the purpose of determining the therapeutically effective or prophylactically effective dosage amount needed to prevent, counter, or arrest the progress of the condition. It is expected that the compound will be administered chronically on a daily basis for a length of time appropriate to treat or prevent the medical condition relevant to the patient, including a course of therapy lasting days, months, years or the life of the patient.

In general, a daily dose of approximately 0.001 to 100 mg/kg, preferably 0.001 to 30 mg/kg, in particular 0.001 to 10 mg/kg (in each case mg per kg of bodyweight) is appropriate for administration to an adult weighing approximately 75 kg in order to obtain the desired results. The daily dose is preferably administered in a single dose or can be divided into several, for example two, three or four individual doses, and may be, for example but not limited to, 0.1 mg, 0.25 mg, 0.5 mg, 0.75 mg, 1 mg, 1.25 mg, 2 mg, 2.5 mg, 5 mg, 10 mg, 20 mg, 40 mg, 50 mg, 75 mg, 100 mg, 125 mg, 150 mg, 175 mg, 200 mg, etc., on a daily basis. In some cases, depending on the potency of the compound or the individual response, it may be necessary to deviate upwards or downwards from the given daily dose. Furthermore, the compound may be formulated for immediate or modified release such as extended or controlled release.

The term “patient” includes animals, preferably mammals and especially humans, who use the instant active agents for the prophylaxis or treatment of a medical condition. Administering of the drug to the patient includes both self-administration and administration to the patient by another person. The patient may be in need of treatment for an existing disease or medical condition, or may desire prophylactic treatment to prevent or reduce the risk for developing said disease or medical condition or developing long-term complications from a disease or medical condition.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. A prophylactically effective amount is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. The terms “preventing,” “prevention,” “prophylactic” and derivatives of these terms as used herein refer to administering a compound to a patient before the onset of clinical symptoms of a condition not yet present in the patient. It is understood that a specific daily dosage amount can simultaneously be both a therapeutically effective amount, e.g., for treatment of hypertension, and a prophylactically effective amount, e.g., for prevention or reduction of risk of myocardial infarction or prevention or reduction of risk for complications related to hypertension.

In the methods of treatment of this invention, the ROMK inhibitors may be administered via any suitable route of administration such as, for example, orally, parenterally, or rectally in dosage unit formulations containing conventional non-toxic pharmaceutically acceptable carriers, adjuvants and vehicles. The term parenteral as used herein includes subcutaneous injections, intravenous (IV), intramuscular, intrasternal injection or infusion techniques. Oral formulations are preferred for treatment of chronic indications such as hypertension or chronic heart failure, particularly solid oral dosage units such as pills, tablets or capsules, and more particularly tablets. IV dosing is preferred for acute treatment, for example for the treatment of acute heart failure.

This invention also provides pharmaceutical compositions comprised of a compound of Formula I and a pharmaceutically acceptable carrier which is comprised of one or more excipients or additives. An excipient or additive is an inert substance used to formulate the active drug ingredient. For oral use, the pharmaceutical compositions of this invention containing the active ingredient may be in forms such as pills, tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions. Tablets contain the active ingredient in admixture with non-toxic pharmaceutically acceptable excipients which are suitable for the manufacture of tablets. The excipients may be for example, inert diluents, such as calcium carbonate, sodium carbonate, lactose, mannitol, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc.

Pharmaceutical compositions may also contain other customary additives, for example but not limited to, wetting agents, stabilizers, emulsifiers, dispersants, preservatives, sweeteners, colorants, flavorings, aromatizers, thickeners, buffer substances, solvents, solubilizers, agents for achieving a depot effect, salts for altering the osmotic pressure, coating agents or antioxidants. Oral immediate-release and time-controlled release dosage forms may be employed, as well as enterically coated oral dosage forms. Tablets may be uncoated or they may be coated by known techniques for aesthetic purposes, to mask taste or for other reasons. Coatings can also be used to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be preserved by the addition of an anti-oxidant such as ascorbic acid. Syrups and elixirs may be formulated with sweetening agents, for example glycerol, propylene glycol, sorbitol or sucrose.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier. Furthermore, a therapeutically effective amount of a compound of this invention can be used for the preparation of a medicament useful for inhibiting ROMK, for causing diuresis and/or natriuresis, and/or for treating, preventing or reducing the risk for any of the medical conditions described herein, in dosage amounts described herein.

The amount of active compound of Formula I and/or its pharmaceutically acceptable salts in the pharmaceutical composition may be, for example but not limited to, from about 0.1 mg to 1 g, particularly 0.1 mg to about 200 mg, more particularly from about 0.1 mg to about 100 mg, and even more particularly from about 0.1 to about 50 mg, per dose on a free acid/free base weight basis, but depending on the type of the pharmaceutical composition, potency of the active ingredient and/or the medical condition being treated, it could also be lower or higher. Pharmaceutical compositions usually comprise about 0.5 to about 90 percent by weight of the active compound on a free acid/free base weight basis.

The compounds of Formula I inhibit ROMK. Due to this property, apart from use as pharmaceutically active compounds in human medicine and veterinary medicine, they can also be employed as a scientific tool or as aid for biochemical investigations in which such an effect on ROMK is intended, and also for diagnostic purposes, for example in the in vitro diagnosis of cell samples or tissue samples. The compounds of Formula I can also be employed as intermediates for the preparation of other pharmaceutically active compounds.

One or more additional pharmacologically active agents may be administered in combination with a compound of Formula I. The additional active agent (or agents) is intended to mean a medicinal compound that is different from the compound of Formula I, and which is a pharmaceutically active agent (or agents) that is active in the body, including pro-drugs, for example esterified forms, that convert to pharmaceutically active form after administration, and also includes free-acid, free-base and pharmaceutically acceptable salts of said additional active agents when such forms are sold commercially or are otherwise chemically possible. Generally, any suitable additional active agent or agents, including but not limited to anti-hypertensive agents, additional diuretics, anti-atherosclerotic agents such as a lipid modifying compound, anti-diabetic agents and/or anti-obesity agents may be used in any combination with the compound of Formula I in a single dosage formulation (a fixed dose drug combination), or may be administered to the patient in one or more separate dosage formulations which allows for concurrent or sequential administration of the active agents (co-administration of the separate active agents). Examples of the one or more additional active agents which may be employed include but are not limited to thiazide-like diuretics, e.g., hydrochlorothiazide (HCTZ or HCT); angiotensin converting enzyme inhibitors (e.g, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril); dual inhibitors of angiotensin converting enzyme (ACE) and neutral endopeptidase (NEP) such as omapatrilat, sampatrilat and fasidotril; angiotensin II receptor antagonists, also known as angiotensin receptor blockers or ARBs, which may be in free-base, free-acid, salt or pro-drug form, such as azilsartan, e.g., azilsartan medoxomil potassium (EDARBI®), candesartan, e.g., candesartan cilexetil (ATACAND®), eprosartan, e.g., eprosartan mesylate (TEVETAN®), irbesartan (AVAPRO®), losartan, e.g., losartan potassium (COZAAR®), olmesartan, e.g, olmesartan medoximil (BENICAR®), telmisartan (MICARDIS®), valsartan (DIOVAN®), and any of these drugs used in combination with a thiazide-like diuretic such as hydrochlorothiazide (e.g., HYZAAR®, DIOVAN HCT®, ATACAND HCT®), etc.); potassium sparing diuretics such as amiloride HCl, spironolactone, epleranone, triamterene, each with or without HCTZ; carbonic anhydrase inhibitors, such as acetazolamide; neutral endopeptidase inhibitors (e.g., thiorphan and phosphoramidon); aldosterone antagonists; aldosterone synthase inhibitors; renin inhibitors (e.g enalkrein; RO 42-5892; A 65317; CP 80794; ES 1005; ES 8891; SQ 34017; aliskiren (2(S),4(S),5(S),7(S)—N-(2-carbamoyl-2-methylpropyl)-5-amino-4-hydroxy-2,7-diisopropyl-8-[4-methoxy-3-(3-methoxypropoxy)-phenyl]-octanamid hemifumarate) SPP600, SPP630 and SPP635); endothelin receptor antagonists; vasodilators (e.g. nitroprusside); calcium channel blockers (e.g., amlodipine, nifedipine, verapamil, diltiazem, felodipine, gallopamil, niludipine, nimodipine, nicardipine, bepridil, nisoldipine); potassium channel activators (e.g., nicorandil, pinacidil, cromakalim, minoxidil, aprilkalim, loprazolam); sympatholitics; beta-adrenergic blocking drugs (e.g., acebutolol, atenolol, betaxolol, bisoprolol, carvedilol, metoprolol, metoprolol tartate, nadolol, propranolol, sotalol, timolol); alpha adrenergic blocking drugs (e.g., doxazocin, prazocin or alpha methyldopa); central alpha adrenergic agonists; peripheral vasodilators (e.g. hydralazine); nitrates or nitric oxide donating compounds, e.g. isosorbide mononitrate; lipid lowering agents, e.g., HMG-CoA reductase inhibitors such as simvastatin and lovastatin which are marketed as ZOCOR® and MEVACOR® in lactone pro-drug form and function as inhibitors after administration, and pharmaceutically acceptable salts of dihydroxy open ring acid HMG-CoA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), and fluvastatin (particularly the sodium salt sold in LESCOL®); a cholesterol absorption inhibitor such as ezetimibe (ZETIA®), and ezetimibe in combination with any other lipid lowering agents such as the HMG-CoA reductase inhibitors noted above and particularly with simvastatin (VYTORIN®) or with atorvastatin calcium;) and/or with an HMG-CoA reductase inhibitor; niacin in immediate-release or controlled release forms, and particularly niacin in combination with a DP antagonist such as laropiprant and/or with an HMG-CoA reductase inhibitor; niacin receptor agonists such as acipimox and acifran, as well as niacin receptor partial agonists; metabolic altering agents including insulin sensitizing agents and related compounds for the treatment of diabetes such as biguanides (e.g., metformin), meglitinides (e.g., repaglinide, nateglinide), sulfonylureas (e.g., chlorpropamide, glimepiride, glipizide, glyburide, tolazamide, tolbutamide), thiazolidinediones also referred to as glitazones (e.g., pioglitazone, rosiglitazone), alpha glucosidase inhibitors (e.g., acarbose, miglitol), dipeptidyl peptidase inhibitors, (e.g., sitagliptin (JANUVIA®), alogliptin, vildagliptin, saxagliptin, linagliptin, dutogliptin, gemigliptin), ergot alkaloids (e.g., bromocriptine), combination medications such as JANUMET® (sitagliptin with metformin), and injectable diabetes medications such as exenatide and pramlintide acetate; phosphodiesterase-5 (PDES) inhibitors such as sildenafil (Revatio, Viagra), tadalafil (Cialis, Adcirca) vardenafil HCl (Levitra); or with other drugs beneficial for the prevention or the treatment of the above-mentioned diseases including but not limited to diazoxide; and including the free-acid, free-base, and pharmaceutically acceptable salt forms, pro-drug forms (including but not limited to esters), and salts of pro-drugs of the above medicinal agents where chemically possible. Trademark names of pharmaceutical drugs noted above are provided for exemplification of the marketed form of the active agent(s); such pharmaceutical drugs could be used in a separate dosage form for concurrent or sequential administration with a compound of Formula I, or the active agent(s) therein could be used in a fixed dose drug combination including a compound of Formula I.

Several methods for preparing the compounds of this invention are described in the following Schemes and Examples. Starting materials and intermediates are purchased, made from known procedures, or as otherwise illustrated. Some frequently applied routes to the compounds of Formula I are also described by the Schemes as follows. In some cases the order of carrying out the the steps of reaction schemes may be varied to facilitate the reaction or to avoid unwanted reaction products. The “R” substituents in the Schemes correspond to the substituents defined in Formula I at the same positions on the structures. The structures:

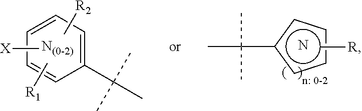

where X is tetrazole, lactone or nitrile, correspond to X and Z respectively in Formula I.

Several methods for preparing the compounds of this invention are described in the examples. Starting materials and intermediates are purchased, made from known procedures, or as otherwise illustrated. Some frequently applied routes to the compounds of Formula I are also described by the Schemes as follows. In some cases the order of carrying out the steps of reaction schemes may be varied to facilitate the reaction or to avoid unwanted reaction products.

Compounds of Formula IA, which is substituted at the benzylic position with an OH group, can be prepared following the sequence outlined in Scheme 1. Coupling of epoxide 1 to spirocyclic amines 2 at elevated temperatures leads to the formation of alcohols IA (Nomura, Y. et al. Chemical & Pharmaceutical Bulletin, 1995, 43(2), 241-6). The reaction can be carried out with conventional heating or by heating using a microwave apparatus. A number of solvents can be used in this reaction; for example, ethanol and 2-propanol. Spirocyclic amines may be free bases, or they may be salts, in which case a base such as triethylamine or N,N′-diisopropylethylamine may be added. When enantiopure chiral epoxides are employed (such as (R)-1 in Scheme 1) epoxide opening occurs with retention of stereochemistry in the benzylic position and individual isomer (R)-IA may be obtained (and if the (S)-epoxide is employed the alcohol produced will have the opposite stereochemistry to that shown). Alternatively, chiral HPLC separation of enantiomers or diastereomers of IA may be performed to provide single enantiomers or diastereomers.

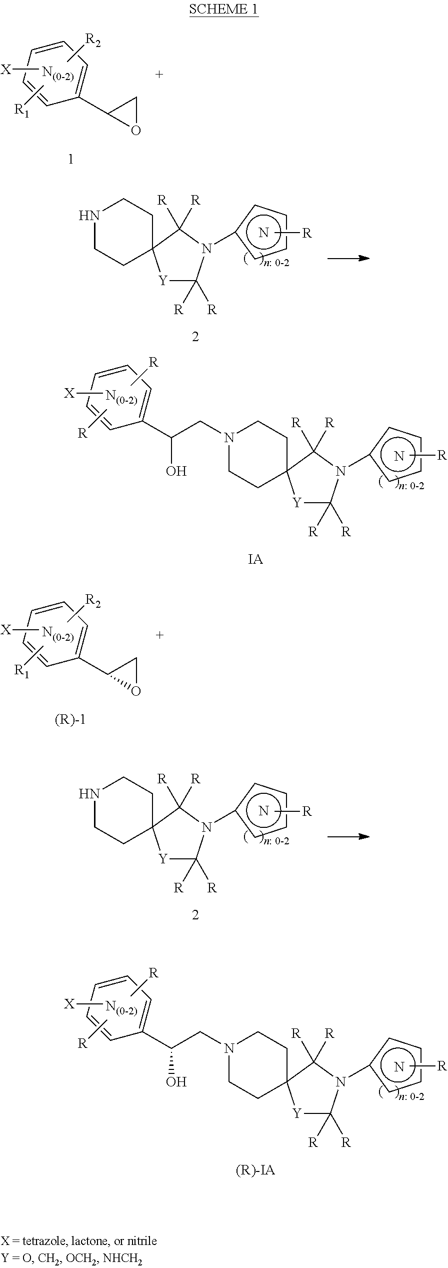

Compounds of Formula IB, which has no OH group at the benzylic position, can be prepared by the sequences detailed in Scheme 2. Aldehydes 3 may be used in reductive alkylation reactions of spirocyclic amines 2 to afford compounds of the invention corresponding to formula IB by using various reductive amination conditions (for example using sodium cyanoborohydride, sodium triacetoxy borohydride, or titanium tetra-isopropoxide, followed by sodium borohydride or sodium cyanoborohydride). Compounds of Formula IB can also be prepared by hydroamination between styrene 4 and spirocyclic amines 2 catalyzed by bis(1,5-cyclooctadiene)rhodium(I) tetrafluoroborate and bis(2-diphenylphosphino-phenyl)ether.

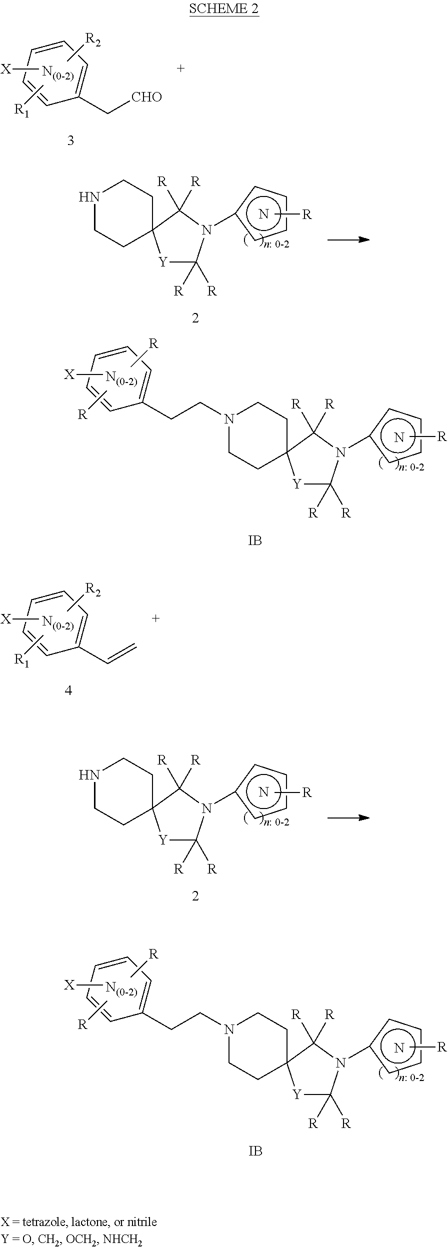

Alternatively, compounds of the Formula IA may be prepared by coupling of the NH in spirocycles 5 to an aromatic or heterocyclic coupling partner 6 (where A represents chloride, bromide, iodide, fluoride, boronic acid). This coupling reaction may be accomplished in a variety of ways, depending upon the nature of of 4 and the coupling partner. For example, in some cases this coupling can be achieved by thermal or microwave heating in one of a variety of potential solvents, such as DMF or dioxane, in the presence or absence of a base such as triethylamine or potassium carbonate, or cesium carbonate. Alternatively, the coupling can be accomplished using a catalyst-ligand system, for example heating with Xantphos and Pd₂(dba)₃ in the presence of a base such as cesium carbonate in a solvent such as dioxane (Buchwald, S. L.; Yin, J. J. Am. Chem. Soc. 2002, 124, 6043). Numerous other C—N coupling conditions, known from the literature such as Pd-catalyzed (Review: Buchwald, S. L. Chem. Sci. 2011, 2, 27) and Cu(I)-catalyzed reactions (Buchwald, S. L. et al. J. Am. Chem. Soc. 2002, 124, 7421), may be applied.

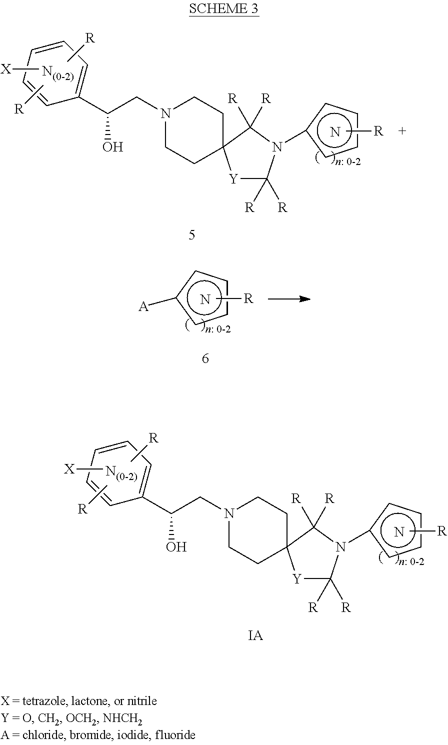

The epoxides 1 (and single enatiomers (R)-1 and (S)-1) can be prepared following the method detailed in Scheme 4. Treatment of 7 (where halide is chloride, bromide, iodide, or trifluoromethane sulfonate) with commercially available potassium vinyl trifluoroborate (Molander, G.; Luciana, A. Journal of Organic Chemistry, 2005, 70, 3950-3956) under palladium catalyzed coupling conditions with an appropriate phosphine ligand gives rise to styrene 4 (Molander, G.; Brown, A. Journal of Organic Chemistry, 2006, 71, 9681-9686). Alternatively, other methods may be employed, for example, using vinylstannane reagents and palladium catalysis. The resulting styrenes 4 is the coupling partner used in Scheme 2. It can be converted to the corresponding epoxides 1 under various epoxidation conditions, for example, with mCPBA (Fringuelli, F. et al. Organic Preparations and Procedures International, 1989, 21, 757-761). The racemic epoxide 1 can be resolved under chiral HPLC chromatography conditions to afford its enantiomers, which can be used in place of 1 according to Scheme 1.

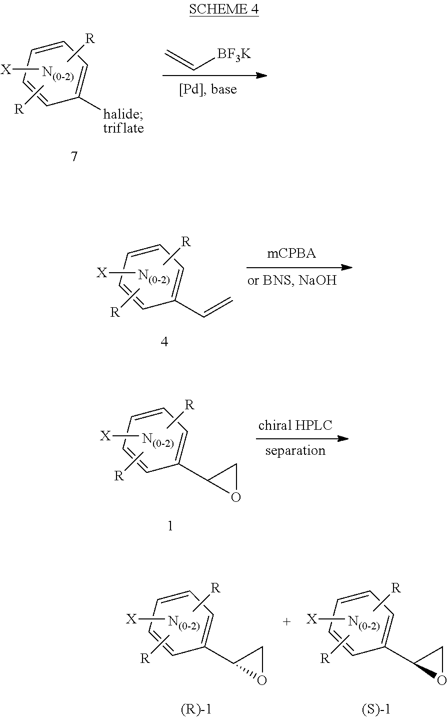

Alternatively, enantiopure epoxides (R)-1 or (S)-1 can be prepared as shown in Scheme 5. Treatment of 7 (where halide is bromide, iodide, or trifluoromethane sulfonate) with commercial available vinyl butylether 7 under palladium catalyzed conditions with a suitable ligand and base (for example Pd(OAc)₂, DPPP, Et₃N) can provide the enol ethers 9. Enol ethers may be prepared using other methods known to the chemist. Treatment of the resulting enol ethers 9 with NBS or other similar reagents affords the corresponding bromomethyl ketones 10. These can be subjected to a variety of asymmetric ketone reduction conditions, for example with an enzyme that can affect such a transformation with high enantioselectivity. Subsequent treatment with a base such as triethylamine leads to cyclization, affording the enantioenriched epoxides (R)-1 or (S)-1 (depending upon the asymmetric reducing agent).

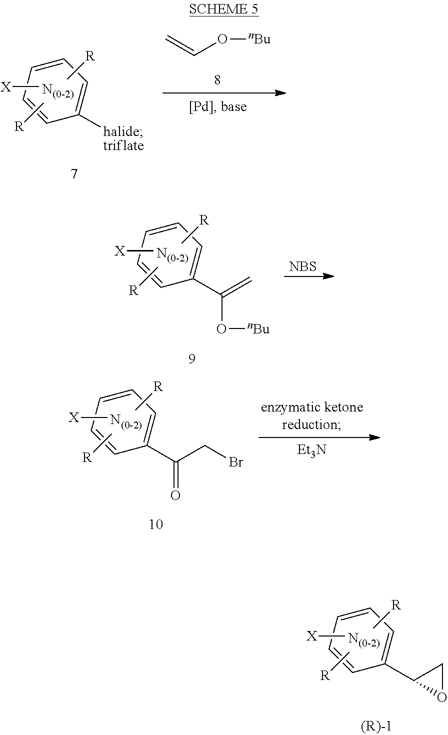

As shown in Scheme 6, aldehyde 3 can be prepared by hydrogenation of intermediate epoxides (1, from Scheme 4) followed by oxidation with Dess-Martine periodinane.

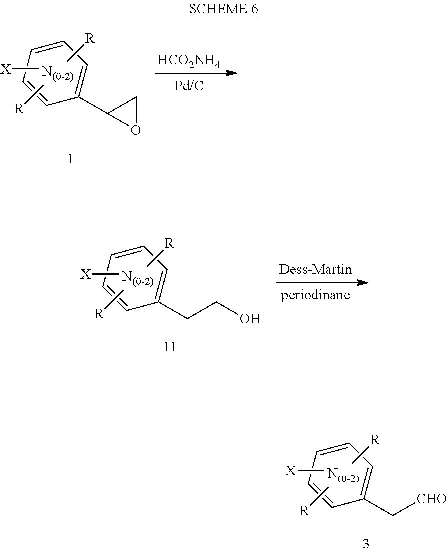

Amine 5 can be prepared in sequences described in Scheme 7. Treatment of epoxide (R)-1 with commercially available or unavailable amines 12 (commercially unavailable amines 12 are prepared as described in the experimental section below) under conventional or microwave heating conditions leads to form 13, which was then deprotected by TFA or HCl to give free amine 5 after treatment with ion-exchange column.

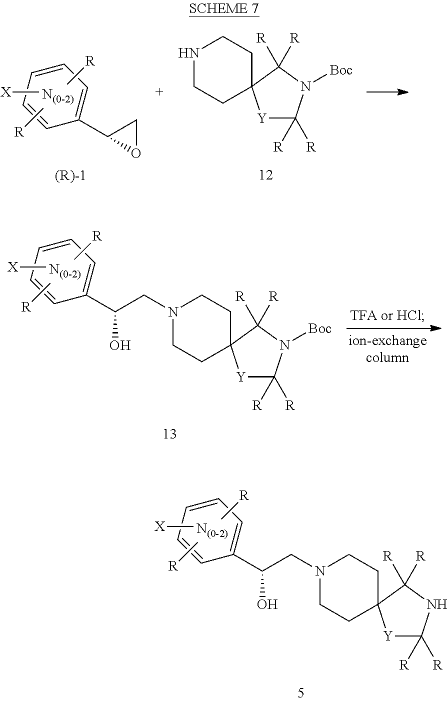

When spirolactams (where R represents a carbonyl group) are involved, amine 5 can be prepared through Scheme 8. Boc deprotection of spirolactams 14 with TFA or HCl, followed by treatment with ion-exchange column affords free amines 15. Epoxide opening reaction of (R)-1 by 15 under conventional or microwave heating conditions leads to amine 5.

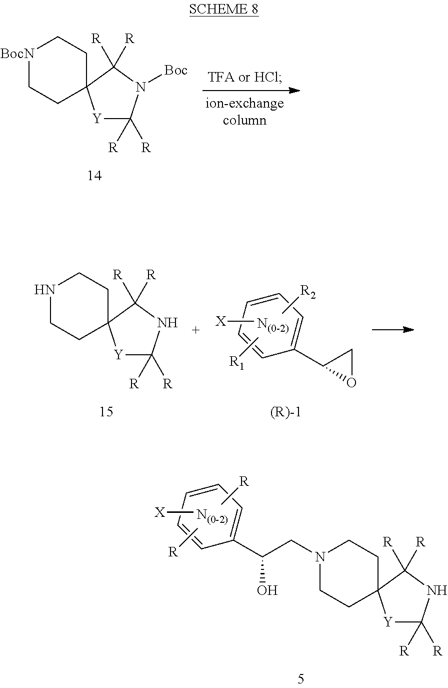

Spirocyclic amines 2 can be prepared as described in Scheme 9. Spirocyclic diamines or amino lactams (where R represents a carbonyl group) 14, protected with Boc group, can be coupled to electrophiles 6 (where A represents I, Cl, Br or OTf) in a variety of ways, depending upon the nature of coupling partners. For example, in some cases this coupling can be achieved by thermal or microwave heating in the presence or absence of a base, such as DIPEA. Sometime the coupling can be achieved using a catalyst-ligand system, for example Pd₂(dba)₃ and Xantphos. Some spirocyclic diamines or amino lactams 14 described herein are commercially available; others can prepared as described in the experimental section below. Some halides 6 are commercially available; some can be prepared as described in the examples below. Intermediates 16 are converted to spirocyclic amines 2 by removal of the protective group; for example, tert-butoxycarbonyl can be removed with TFA or HCl.

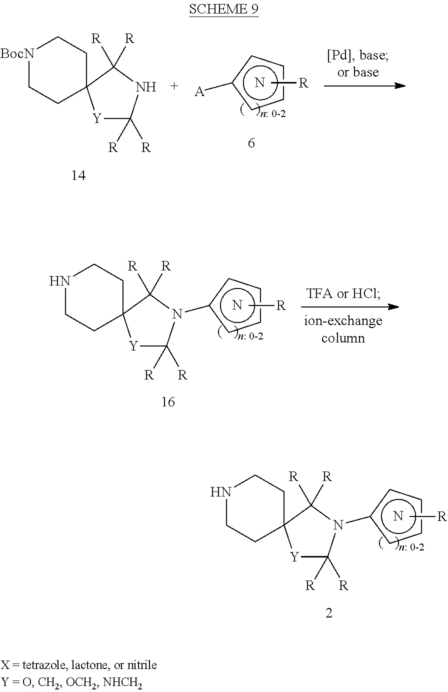

### General Procedures:

Reactions sensitive to moisture or air were performed under nitrogen or argon using anhydrous solvents and reagents. The progress of reactions was determined by either analytical thin layer chromatography (TLC) usually performed with E. Merck pre-coated TLC plates, silica gel 60F-254, layer thickness 0.25 mm or liquid chromatography-mass spectrometry (LC-MS). Typically the analytical LC-MS system used consisted of a Waters ZQ™ platform with electrospray ionization in positive ion detection mode with an Agilent 1100 series HPLC with autosampler. The column was usually a Water Xterra MS C18, 3.0×50 mm, 5 μm. The flow rate was 1 mL/min, and the injection volume was 10 μL. UV detection was in the range 210-400 nm. The mobile phase consisted of solvent A (water plus 0.06% TFA) and solvent B (acetonitrile plus 0.05% TFA) with a gradient of 100% solvent A for 0.7 min changing to 100% solvent B over 3.75 min, maintained for 1.1 min, then reverting to 100% solvent A over 0.2 min. Preparative HPLC purifications were usually performed using a mass spectrometry directed system. Usually they were performed on a Waters Chromatography Workstation configured with LC-MS System Consisting of: Waters ZQ™ single quad MS system with Electrospray Ionization, Waters 2525 Gradient Pump, Waters 2767 Injecto/Collector, Waters 996 PDA Detector, the MS Conditions of: 150-750 amu, Positive Electrospray, Collection Triggered by MS, and a Waters SUNFIRE® C-18 5 micron, 30 mm (id)×100 mm column. The mobile phases consisted of mixtures of acetonitrile (10-100%) in water containing 0.1% TFA. Flow rates were maintained at 50 mL/min, the injection volume was 1800 μL, and the UV detection range was 210-400 nm. Mobile phase gradients were optimized for the individual compounds. Reactions performed using microwave irradiation were normally carried out using an Emrys Optimizer manufactured by Personal Chemistry, or an Initiator manufactured by Biotage. Concentration of solutions was carried out on a rotary evaporator under reduced pressure. Flash chromatography was usually performed using a Biotage® Flash Chromatography apparatus (Dyax Corp.) on silica gel (32-63 mM, 60 Å pore size) in pre-packed cartridges of the size noted. ¹H NMR spectra were acquired at 500 MHz spectrometers in CDCl₃ solutions unless otherwise noted. Chemical shifts were reported in parts per million (ppm). Tetramethylsilane (TMS) was used as internal reference in CD₃Cl solutions, and residual CH₃OH peak or TMS was used as internal reference in CD₃OD solutions. Coupling constants (J) were reported in hertz (Hz). Chiral analytical chromatography was performed on one of CHIRALPAK® AS, CHIRALPAK®AD, CHIRALCEL® OD, CHIRALCEL® IA, or CHIRALCEL® OJ columns (250×4.6 mm) (Daicel Chemical Industries, Ltd.) with noted percentage of either ethanol in hexane (% Et/Hex) or isopropanol in heptane (% IPA/Hep) as isocratic solvent systems. Chiral preparative chromatography was conducted on one of of CHIRALPAK AS, of CHIRALPAK AD, CHIRALCEL® OD, CHIRALCEL®IA, CHIRALCEL® OJ columns (20×250 mm) (Daicel Chemical Industries, Ltd.) with desired isocratic solvent systems identified on chiral analytical chromatography or by supercritical fluid (SFC) conditions.

Abbreviations and acronyms that may be used herein include: —C(O)CH₃ (Ac); —OC(O)CH₃ (OAc); acetic acid (AcOH; HOAc); 1-chloroethylchloroformate (ACE-Cl); 2,2′-bis(diphenylphosphino)-1,1′-binaphthyl (BINAP); benzyl (Bn); t-butyloxycarbonyl (Boc or BOC); di-t-butyl dicarbonate ((BOC)₂O, Boc₂O); benzyloxycarbonyl (Cbz); n-butyl (Bu); tert-butyl (t-butyl); cyclopentyl methyl ether (CPME); carbonyldiimidazole (CDI); diethylaminosulfur trifluoride (DAST); dibenzylideneacetone (dba); 1,8-diazabicyclo[5.4.0]undec-7-ene (DBU); 1,2-dichloroethane (DCE); dichloromethane (DCM); diethyl amine (DEA); dimethoxyethane (DME); diisobutylaluminium hydride (DIBAL-H); N,N-diisopropylethylamine (DIEA, DIPEA, Hunig's base); dioxane is 1,4-dioxane; di-isopropylamine (DIPA); 1,1′-bis(diphenylphosphino)ferrocene (dppf, DPPF); Dess-Martin Periodinane (DMP; 1,1,1-triacetoxy-1,1-dihydro-1,2-benziodoxol-3(1H)-one); dimethylsulfide (DMS); dimethylsulfoxide (DMSO); N;N-dimethylformamide (DMF); 4-dimethylaminopyridine (DMAP); dimethylacetamide (DMA; DMAC); 1,3-bis(diphenylphosphino)propane (DPPP); (Oxydi-2,1-phenylene)bis(diphenylphosphine) (DPEPhos); eithyl (ET); ethyl acetate (EtOAc or EA); ethanol (EtOH); diethyl ether (ether or Et₂O); 1-ethyl-3-(3-dimethylaminopropyl)carbodiimide (EDC, EDAC or EDCI); 2-(7-Aza-1H-benzotriazole-1-yl)-1,1,3,3-tetramethyluronium hexafluorophosphate (HATU); hexane (Hex); hexamethylphosphoramide (HMPA); 1-hydroxybenzotriazole hydrate (HOBt); isopropanol (IPA or iPrOH); isopropyl acetate (IPAc); potassium bis(trimethylsilyl)amide (KHMDS); lithium aluminum hydride (LAH); lithium diisopropylamide (LDA); 3-chloroperoxybenzoic acid (mCPBA); methanol (MeOH); CH₃SO₂— (mesyl or Ms); methane sulfonyl chloride or mesyl chloride (MsCl); methanesulfonic acid (MsOH); methyl (Me); methyl tert-butyl ether (MTBE); nicotinamide adenine dinucleotide phosphate (NADP); N-bromo succinimide (NBS); N-chlorosuccinimide (NCS); N-iodosuccinimide (NIS); N-methylmorpholine-N-oxide (NMO); N-methyl morpholine (NMP); sodium hexamethyldisilazide (NaHMDS); sodium triacetoxyborohydride (NaBH(OAc)₃); pyridinium chlorochromate (PCC); phenyl (Ph); petroleum ether (PE or petrol ether); tetrakis(triphenylphosphine)palladium (Pd(PPh₃)₄); tris(dibenzylidineacetone)dipalladium (Pd₂(dba)₃); Pd(dppf)Cl₂ or PdCl₂(dppf) is 1,1′-Bis(diphenylphosphino)ferrocene]-dichloropalladium(II) which may be complexed with CH₂Cl₂; tetra-n-butylammonium fluoride (TBAF); tert-butyldimethylsilyl chloride (TBS-Cl); triethylamine (TEA); trifluoroacetic acid (TFA); —SO₂CF₃ (TO; trifluoromethanesulfonic acid (triflic acid, TfOH); trifluoromethanesulfonic anhydride (triflic anhydride, (Tf)₂O); 2-tetrahydrofuran (THF); N,N,N′,N′-tetramethylethylenediamine (TMEDA); p-toluenesulfonic acid (TsOH or PTSA); dicyclohexylphosphino-2′,4′,6′-triisopropylbiphenyl (X-Phos); diethylaminodifluorosulfinium tetrafluoroborate (XtalFluor-E®); 4,5-bis(diphenylphosphino)-9,9-dimethylxanthene (Xantphos). Additional abbreviations and acronyms are: racemic or racemate (rac.); starting material (SM); round-bottom flask (RB or RBF); aqueous (aq); saturated aqueous (sat'd); saturated aqueous sodium chloride solution (brine); maximum temperature (Tₘₐₓ); medium pressure liquid chromatography (MPLC); high pressure liquid chromatography (HPLC); preparative HPLC (prep-HPLC); flash chromatography (FC); liquid chromatography (LC); supercritical fluid chromatography (SFC); 2-dicyclohexylphosphino-2′,6′-dimethoxybiphenyl (SPhos); thin layer chromatography (TLC); preparative TLC (prep-TLC); mass spectrum (ms or MS); liquid chromatography-mass spectrometry (LC-MS, LCMS or LC/MS); column volume (CV); room temperature (rt, r.t. or RT); hour(s) (h or hr); minute(s) (min); retention time (Rₜ); gram(s) (g); milligram(s) (mg); milliliter(s) (mL); microliter(s) (μL); millimole (mmol); volume:volume (V/V). CELITE® is a trademark name for diatomaceous earth, and SOLKA FLOC® is a trademark name for powdered cellulose. X or x may be used to express the number of times an action was repeated (e.g., washed with 2×200 mL 1N HCl), or to convey a dimension (e.g., the dimension of a column is 30×250 mm).

The following are representative procedures for the preparation of the compounds used in the following Examples, or which can be substituted for the compounds used in the following Examples which may not be commercially available.

### Intermediate 1

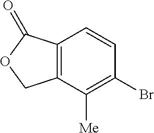 [SMILES: Cc1c(Br)ccc2c1COC2=O]

### Step A: (3-bromo-2-methylphenyl)methanol

To a solution of 3-bromo-2-methyl benzoic acid (35 g, 163 mmol) in THF (200 mL) was added borane THF complex (1.0 M, 212 mL, 212 mmol). The mixture was allowed to stir for 24 h. TLC showed one single product spot. The reaction was quenched with water. The solvent THF was removed under reduced pressure. The resulting solid was dissolved in ethyl acetate (500 mL), washed with 1N HCl, sodium carbonate, and brine. The organic layer was dried over sodium sulfate and concentrated to afford (3-bromo-2-methylphenyl)methanol. ¹H NMR (500 MHz, CDCl₃) δ 7.76 (d, J=8.0 Hz, 1H), 7.63 (d, J=8.0 Hz, 1H), 5.30 (s, 2H), 2.42 (s, 3H).

### Step B: 5-bromo-4-methyl-2-benzofuran-1(3H)-one

To a flask charged with (3-bromo-2-methylphenyl)methanol (6.0 g, 30 mmol) was added a 1M TFA solution of thallium trifluoroacetate (16.2 g, 29.8 mmol). The mixture was stirred at RT overnight. Analysis by TLC showed no starting material remaining. The solvent was removed under vacuum, and the residue was pumped under high vacuum for 30 min to ensure complete removal of TFA. To the residue was then added palladium(II) chloride (529 mg, 2.98 mmol), lithium chloride (2.53 g, 59.7 mmol), magnesium oxide (2.41 g, 59.7 mmol), and MeOH (150 mL). The reaction was flushed with CO twice, and kept under CO at room temperature. Analysis by LC showed a big product spot within 2 hours. To this solution was added ethyl acetate to precipitate the salts. The black solution was filtered through a CELITE® pad, washed with EtOAc, adsorbed onto silica and purified by silica gel chromatography to afford 5-bromo-4-methyl-2-benzofuran-1(3H)-one. ¹H NMR (500 MHz, CDCl₃) δ ppm 7.71 (d, J=8.0 Hz, 1H), 7.58 (d, J=8.0 Hz, 1H), 5.25 (s, 2H), 2.37 (s, 3H).

### Intermediate 2

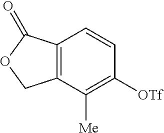 [SMILES: Cc1c(OS(=O)(=O)C(F)(F)F)ccc2c1COC2=O]

### Step A: 3-hydroxymethyl-2-methyl phenol

To a 5 L 3-neck round bottomed flask equipped with overhead stirrer was charged NaBH₄ (87.0 g, 2.30 mol) and THF (3.0 L) and the resulting slurry was cooled to 10° C. To the slurry was then added 3-hydroxy-2-methyl benzoic acid (175 g, 1.15 mol) portionwise over 20 min (T_{Max} 17° C.). A stirrable slurry formed, and was aged for an additional 45 min at 10-15° C. after which BF₃—OEt₂ (321 mL, 2.53 mol) was added slowly over 1.5 hours. The slurry was aged at 10° C. to 15° C. for 2 h then assayed for reaction completion (98.5% conversion). The slurry was cooled to less than 10° C. and quenched with 931 mL MeOH slowly over 1.5 h (gas evolution). The resulting slurry was aged overnight at RT. The batch was cooled to less than 10° C. then quenched with 1 N HCl (1.5 L) to get a homogeneous solution (pH solution ˜1), which was aged for 30 min and then the organic solvents were removed by rotary evaporation to approximately 1.8 L of total reaction volume (bath temperature was set to 50° C.; internal temperature of concentrate after rotary evaporation was ˜40° C.). The slurry was held at 45° C. for 30 min then cooled slowly to 15° C. The solids were filtered and washed with cold (15° C.) water (2×300 mL), providing 3-hydroxymethyl-2-methyl phenol. ¹H-NMR (400 MHz, DMSO-d₆): δ 9.11 (s, 1H), 6.95 (t, J=7.8 Hz, 1H), 6.82 (d, J=7.4 Hz, 1H), 6.71 (d, J=7.8 Hz, 1H), 4.93 (t, J=5.5 Hz, 1H), 4.44 (d, J=5.5 Hz, 2H), 2.06 (s, 3H).

### Step B: 4-Bromo-3-hydroxymethyl-2-methyl phenol

3-Hydroxymethyl-2-methyl phenol (113.9 g, 824.0 mmol) was dissolved in a mixture of acetonitrile (850 mL) and trifluoroacetic acid (750.0 mL, 9,735 mmol) in a 3-neck 5-L flask under nitrogen. The reaction mixture was cooled to −33° C. N-bromosuccinimide (141 g, 791 mmol) was added over 15 minutes, with the temperature during addition in the range of −35 to −33° C. The reaction mixture was allowed to stir for an additional 15 min during which time the temperature decreased to −40° C. The cooling bath was removed, and potassium carbonate (741.0 g, 5,358 mmol) diluted with water to a total of 1.0 L was added. Off-gassing was observed, and the temperature increased to 25° C. MTBE (1.5 L) was added, and the reaction mixture was transferred to a reparatory funnel. The layers were separated. The aqueous layer was diluted with water (500 mL) and extracted with MTBE (1 L)+EtOAc (500 mL), and then MTBE (500 mL)+EtOAc (250 mL). The combined organic layers were washed with water (240 mL) and dried over sodium sulfate. The sodium sulfate was removed by filtration, washed with additional MTBE and concentrated under reduced pressure. MTBE (684 mL, 2 volumes) was added, and the suspension was heated to 40° C. to produce a homogeneous solution. The solution was allowed to cool to room temperature. Six volumes of heptane were added, and the suspension was stirred overnight. The suspension was filtered, and the crystals were washed with 4:1 heptane: MTBE (500 mL), followed by heptane (500 mL). The solid was dried under vacuum, providing 4-bromo-3-hydroxymethyl-2-methyl phenol. ¹H NMR (400 MHz, DMSO-d₆): δ 9.52 (s, 1H), 7.21 (d, J=8.6 Hz, 1H), 6.71 (d, J=8.6 Hz, 1H), 4.88 (t, J=5.1 Hz, 1H), 4.59 (d, J=5.1 Hz, 2H), 2.23 (s, 3H).

### Step C: 5-Hydroxy-4-methyl-3H-isobenzofuran-1-one

To a 2 L 3 neck flask equipped with overhead stirrer, N₂ inlet, and condenser were charged 4-bromo-3-hydroxymethyl-2-methyl phenol (100 g, 461 mmol), CuCN (83.0 g, 921 mmol), and DMF (500 mL). The solution was sparged with N₂ for 15 min then heated to 145° C. to obtain a homogeneous solution. The solution was aged at 145° C. for 2 h, then the reaction mixture was cooled to 95° C. 41.5 mL water was added (sparged with N₂), and the reaction aged for 20 h. The reaction was cooled to RT then the solids filtered through SOLKA-FLOK® and the cake washed with 50 mL DMF. To a 3 L flask containing 1 L EtOAc was added the DMF filtrate. A precipitate coating formed in bottom of flask. The DMF/EtOAc suspension was filtered through SOLKA-FLOK® and the cake was washed with 250 mL EtOAc. The resulting filtrate was washed with 5% brine solution (3×500 mL). The aqueous layers were extracted with 500 mL EtOAc and the combined organics were dried over MgSO₄, filtered and evaporated. The solids were slurried in 250 mL MTBE at RT then filtered and washed with 100 mL MTBE. The solids were dried under vacuum at RT, providing 5-hydroxy-4-methyl-3H-isobenzofuran-1-one. ¹H NMR (400 MHz, DMSO-d₆): δ 10.52 (s, 1H), 7.51 (d, J=8.3 Hz, 1H), 6.99 (d, J=8.3 Hz, 1H), 5.28 (s, 2H), 2.07 (s, 3H).

### Step D: 4-methyl-1-oxo

1,3-dihydroisobenzofuran-5-yl trifluoromethanesulfonate: 5-Hydroxy-4-methyl-3H-isobenzofuran-1-one (46.8 g, 285 mmol) was suspended in dichloromethane (935 mL) in 2-L roundbottom flask equipped with overhead stirrer under nitrogen. Triethylamine (59.5 mL, 427 mmol) was added, and the reaction mixture was cooled in an ice bath to 3.8° C. Trifluoromethanesulfonic anhydride (67.4 mL, 399 mmol) was added via addition funnel over 50 min, keeping the temperature <10° C. After stirring the reaction mixture for an additional 15 min, the reaction mixture was quenched with water (200 mL), then stirred with DARCO® KB (activated carbon, 25 g) for 15 min. The biphasic mixture was filtered over SOLKA-FLOK®, washing with additional dichloromethane, and transferred to a reparatory funnel, whereupon it was diluted with additional water (300 mL). The layers were separated, and the organic layer was washed with water (500 mL) and 10% brine (200 mL). The dichloromethane solution was dried over sodium sulfate, filtered and evaporated. The orange-red solid was adsorbed onto silica gel (27.5 g) and eluted through a pad of silica gel (271 g) with 25% ethyl acetate/hexanes. The resulting solution was concentrated under vacuum with the product crystallizing during concentration. The suspension was filtered, the solid washed with heptane and dried under vacuum and nitrogen, providing trifluoromethanesulfonic acid 4-methyl-1-oxo-1,3-dihydroisobenzofuran-5-yl ester. ¹H NMR (400 MHz, CDCl₃): δ 7.87 (d, J=8.4 Hz, 1H), 7.47 (d, J=8.4 Hz, 1H), 5.32 (s, 2H), 2.41 (s, 3H).

### Intermediate 3

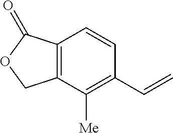

5-Bromo-4-methyl-2-benzofuran-1(3H)-one (598 mg, 4.47 mmol) (INTERMEDIATE 1), potassium vinyl trifluoroborate (507 mg, 2.23 mmmol), PdCl₂(dppf)-CH₂Cl₂ adduct (182 mg, 0.223 mmmol), and TEA (0.622 mL, 4.47 mmol) were added to 10 mL ethanol in a 20 mL microwave tube. The tube was sealed and degassed, then heated to 140° C. for 20 min. Analysis by LC-MS showed product peak. The reaction mixture was diluted with ethyl acetate, washed with brine twice, dried and evaporated to dryness. The crude product was purified by MPLC chromatography using a 120 g RediSep® column and 0-80% EtOAc/hexane solvent system to yield 5-ethenyl-4-methyl-2-benzofuran-1(3H)-one. ¹H NMR (500 MHz, CDCl₃): δ ppm 7.76 (d, J=8 Hz, 1H), 7.03 (dd, J=11, 17 Hz, 1H), 5.84 (d, J=17 Hz, 1H), 5.55 (d, J=11 Hz, 1H), 5.29 (s, 2H), 2.34 (s, 3H); LC-MS: M+1=175.

### Intermediate 4

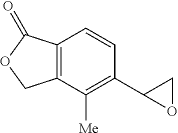 [SMILES: Cc1c(C2CO2)ccc2c1COC2=O]

5-Ethenyl-4-methyl-2-benzofuran-1(3H)-one (1.46 g, 8.38 mmol) (INTERMEDIATE 30) was added to DCM (25 mL) at 0° C. then mCPBA (2.89 g, 16.8 mmol) was added and the mixture was stirred at RT overnight. The reaction mixture was washed once each with saturated aqueous Na₂S₂O₃, NaHCO₃, and brine. The organic layer was dried over Na₂SO₄, filtered, and evaporated to dryness. The crude material was purified by MPLC chromatography through 120 g REDISEP® column eluting with 0-80% EtOAc/hexane solvent system to yield target 4-methyl-5-oxiran-2-yl-2-benzofuran-1(3H)-one. ¹H NMR (500 MHz, CDCl₃): δ ppm 7.77 (d, J=8 Hz, 1H), 7.43 (d, J=8 Hz, 1H), 5.30 (s, 2H), 4.12 (s, 1H), 3.27 (t, J=4 Hz, 1H), 2.735 (dd, J=2.2, 5.5 Hz, 1H), 2.43 (s, 3H). LC-MS: M+1=191.

### Intermediates 4A and 4B (Method 1)

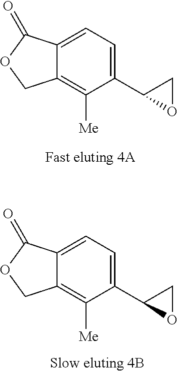 [SMILES: Cc1c([C@@H]2CO2)ccc2c1COC2=O ; Cc1c([C@H]2CO2)ccc2c1COC2=O]

### 4A: 4-methyl-5-[(2R)-oxiran-2-yl]-2-benzofuran-1(3H)-one

### 4B: 4-methyl-5-[(2S)-oxiran-2-yl]-2-benzofuran-1(3H)-one

Racemic 4-methyl-5-oxiran-2-yl-2-benzofuran-1(3H)-one (INTERMEDIATE 4) was resolved on a CHIRALPAK® AD-H column (5×25 cm) under supercritical fluid chromatography (SFC) conditions on a Berger MGIII preparative SFC instrument. The racemate was diluted to 50 mg/mL in 1:1 DCM:MeOH. The separation was accomplished using 10% EtOH/CO₂, flow rate 200 mL/min, 100 bar, 25° C. 500 μl injections were spaced every 2.12 mins. The fast epoxide (4-methyl-5-[(2R)-oxiran-2-yl]-2-benzofuran-1(3H)-one, 4B) eluted first, and the slow epoxide (4-methyl-5-[(2S)-oxiran-2-yl]-2-benzofuran-1(3H)-one, 4A) eluted second.

Alternatively, the resolution could also be achieved using a mobile phase of 8% MeOH/98% CO₂ with a flow rate of 100 mL/min. In that case the sample was prepared by dissolving in methanol, 20 mg/mL, and using a 1 mL volume per injection. After separation, the fractions were dried off via rotary evaporator at bath temperature 40° C.

The absolute stereochemistry of each enantiomer was inferred based on the X-ray crystal structure determination of a final compound made with 4B and by Mosher ester and Trost ester H NMR analysis of esters made starting from 4B. Both epoxide isomers find utility in the present invention.

### Intermediate 4A (Method 2)

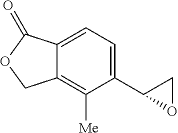 [SMILES: Cc1c([C@@H]2CO2)ccc2c1COC2=O]

### 4-methyl-5-[(2R)-oxiran-2-yl]-2-benzofuran-1(3H)-one

### Step A: 5-(1-Butoxy-vinyl)-4-methyl-3H-isobenzofuran-1-one

To a 1 L 3-neck flask was charged 4-methyl-1-oxo-1,3-dihydroisobenzofuran-5-yl trifluoromethanesulfonate (63.0 g, 213 mmol) (INTERMEDIATE 2), DMF (315 mL), butyl vinyl ether (138 mL, 1063 mmol)) then Et₃N (35.6 mL, 255 mmol). The solution was sparged with N₂ for 20 min. To the solution was added Pd(OAc)₂ (1.19 g., 5.32 mmol) and DPPP (2.41 g., 5.85 mmol) and sparged for an additional 10 min then heated to 80° C. After aging for a 1 hr, the solution was cooled to less than 10° C. then quenched with 630 mL EtOAc and washed with 5% NH₄Cl (2×315 mL), 10% brine (2×315 mL), dried over MgSO₄, filtered, concentrated by rotary evaporation and flushed with EtOAc (3×100 mL) to remove excess butyl vinyl ether, providing crude 5-(1-butoxy-vinyl)-4-methyl-3H-isobenzofuran-1-one. ¹H NMR (400 MHz, DMSO-d₆): δ 7.67 (d, J=7.7 Hz, 1H), 7.48 (d, J=7.7 Hz, 1H), 5.42 (s, 2H), 4.54 (d, J=2.3 Hz, 1H), 4.27 (d, J=2.3 Hz, 1H), 3.85 (t, J=6.4 Hz, 2H), 2.27 (s, 3H), 1.71-1.64 (m, 2H), 1.46-1.37 (m, 2H), 0.92 (t, J=7.4 Hz, 3H).

### Step B: 5-(2-Bromo-acetyl)-4-methyl-3H-isobenzofuran-1-one

To a 1 L 3-neck flask equipped with overhead stirrer was added crude 5-(1-butoxy-vinyl)-4-methyl-3H-isobenzofuran-1-one (55.8 g) and THF (315 mL). The solution was cooled to less than 5° C. after which water (79 mL) was added and the solution was maintained at less than 5° C. NBS (41.6 g) was then added portion-wise while maintaining Tₘₐₓ=19° C. The solution was then warmed to RT for 30 minutes. HBr (48%, 0.241 mL) was added and the reaction was aged at RT for approximately 1 h after which 236 mL water was then added to the batch. A water bath is used to maintain temp at 20° C. Another 315 mL of water was added (solvent composition 1:2 THF:water) and the slurry was cooled to 15° C. The resulting solids were filtered and washed with cold 1:2 THF:water (15° C.): 150 mL displacement wash followed by 100 mL slurry wash. The solids were dried under vacuum at RT to provide 5-(2-bromo-acetyl)-4-methyl-3H-isobenzofuran-1-one. ¹H NMR (400 MHz, DMSO-d₆): δ 7.99 (d, J=7.8 Hz, 1H), 7.82 (d, J=7.8 Hz, 1H), 5.49 (s, 2H), 4.92 (s, 2H), 2.33 (s, 3H).

### Step C: 4-methyl-5-[(2R)-oxiran-2-yl]-2-benzofuran-1(3H)-one

5-(2-Bromo-acetyl)-4-methyl-3H-isobenzofuran-1-one (48.8 g., 181 mmol) was charged to a 5 L 3 neck round bottom equipped with overhead stirrer, thermocouple, and heating mantle. 2-Propanol (1.22 L) was added, followed by 610 mL of pH 7 0.1M potassium phosphate buffer. Buffer solution (610 mL) was charged to a 1.0 L Erlenmeyer flask, and 2.44 g of NADP was added to the Erlenmeyer flask and swirled to dissolve. A reducing enzyme, KRED MIF-20 (2.44 g) (available from Codexis, Inc., 200 Penobscot Drive, Redwood City, Calif. 94063, www.codexis.com, tel. 1-650-421-8100) was added to the Erlenmeyer flask and the mixture was swirled to dissolve the solids. The resulting solution was added to the 5 L round bottom, which was then heated to 28° C. and aged for 6 hours, at which point the reaction was cooled to RT and triethylamine (50.2 mL, 360 mmol) was added. The resulting solution was aged at 40° C. for 1 h. The light slurry solution was cooled to RT, after which 122 g NaCl was added. The solution was aged at RT then extracted with 1.22 L IPAc. The aqueous layer was re-extracted with 400 mL IPAc and the combined organics were washed with 400 mL 20% brine solution, dried over MgSO₄, filtered and concentrated by rotary evaporation. The resulting solids were taken up in 100 mL IPAc (thick slurry). Hexanes were added (400 mL) and the suspension aged at RT then filtered and washed w/5:1 Hexanes:IPAc solution (150 mL). The crystalline solids were dried under vacuum at RT to provide 4-methyl-5-[(2R)-oxiran-2-yl]-2-benzofuran-1(3H)-one. ¹H NMR (400 MHz, CDCl₃): δ 7.75 (d, J=8.1 Hz, 1H), 7.42 (d, J=8.1 Hz, 1H), 5.28 (s, 2H), 4.10 (dd, J=4.0, 2.8, 1H), 3.26 (dd, J=5.6, 4.0, 1H), 2.72 (dd, J=5.6, 2.8, 1H), 2.42 (s, 3H).

### Intermediate 4C

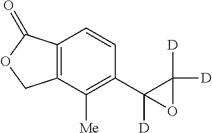 [SMILES: [2H]C1([2H])OC1([2H])c1ccc2c(c1C)COC2=O]

### Step A: 4-Methyl-5-(4,4,5,5-tetramethyl-1,3,2-dioxaborolan-2-yl)isobenzofuran-1(3H)-one

A mixture of 4-methyl-1-oxo-1,3-dihydroisobenzofuran-5-yl trifluoromethanesulfonate (INTERMEDIATE 4B, Step D, 5000 mg, 16.9 mmol), KOAc (2000 mg, 20.3 mmol), Pd(dppf)Cl₂ (618 mg, 0.844 mmol) and bis(pinacolato)diboron (5144 mg, 20.3 mmol) in dioxane (56 mL) was stirred at 75° C. overnight under N₂. The reaction solution was concentrated, and the residue was dissolved in EtOAc (100 mL) and filtered. The filtrate was washed with water (50 mL). The organic layer was separated and concentrated, and the residue was purified by column chromatography (0-100% EtOAc/hex) to give the title compound. LC/MS: [(M+1)]⁺=275; ¹H NMR (500 MHz, CDCl₃) δ 7.96 (d, J=7.7 Hz, 1H), 7.74 (d, J=7.5 Hz, 1H), 5.28 (s, 2H), 2.54 (s, 3H), 1.40 (s, 12H).

### Step B: 4-Methyl-5-vinyl-d₃-isobenzofuran-1(3H)-one

A microwave vial was charged with 4-methyl-5-(4,4,5,5-tetramethyl-1,3,2-dioxaborolan-2-yl)isobenzofuran-1(3H)-one (770 mg, 2.81 mmol), Pd(PPh₃)₂Cl₂ (197 mg, 0.281 mmol), toluene (9363 μl), 20% Na₂CO₃/H₂O (0.6 mL), and vinyl-d₃ bromide (4.21 mL, 1 M in THF, 4.21 mmol). The reaction mixture was stirred at 90° C. for overnight under N₂. The reaction solution was concentrated, and the residue was purified by column chromatography (EtOAc/hex 0-50%) to give 4-methyl-5-vinyl-d₃-isobenzofuran-1(3H)-one. LC/MS: [(M+1)]⁺=178; ¹H NMR (500 MHz, CDCl₃) δ 7.96 (d, J=7.5 Hz, 1H), 7.75 (d, J=7.5 Hz, 1H), 5.29 (s, 2H), 2.54 (s, 3H).

### Step C: 4-Methyl-5-(oxiran-d₃-2-yl)isobenzofuran-1(3H)-one

To 4-methyl-5-vinyl-d₃-isobenzofuran-1(3H)-one (300 mg, 1.69 mmol) in DCM (17 mL) was added mCPBA (584 mg, 3.39 mmol) at 0° C. The reaction mixture was stirred at rt overnight, and washed with saturated NaHCO₃. The organic layer was dried and evaporated to dryness. The crude product was purified by column chromatograph (0-100% EtOAc/hex) to give 4-methyl-5-(oxiran-d₃-2-yl)isobenzofuran-1(3H)-one. LC/MS: [(M+1)]⁺=194; ¹H NMR (500 MHz, CDCl₃) δ 7.61 (d, J=7.9 Hz, 1H), 7.31 (d, J=7.9 Hz, 1H), 5.20 (s, 2H), 2.35 (s, 3H).

### Intermediate 5

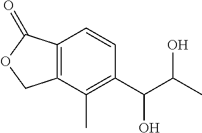 [SMILES: Cc1c(C(O)C(C)O)ccc2c1COC2=O]

### Step A: (E)-4-Methyl-5-(prop-1-en-1-yl)isobenzofuran-1(3H)-one

To Pd(dppf)Cl₂ (0.220 g, 0.338 mmol), K₃PO₄ (6.75 mL, 1 M in water, 6.75 mmol) in THF (22 mL) was added potassium (E)-trifluoro(prop-1-en-1-yl)borate (0.749 g, 5.06 mmol) and 4-methyl-1-oxo-1,3-dihydroisobenzofuran-5-yl trifluoromethanesulfonate (1.0 g, 3.38 mmol). The reaction mixture was de-gassed for 10 min and the resulting mixture was stirred overnight at 70° C. The reaction mixture was cooled to room temperature and diluted with EtOAc and water. After separation of layers, the aqueous layer was extracted with EtOAc, and combined organic layers were washed with brine, dried over anhydrous MgSO₄, filtered, concentrated and purified by silica gel column chromatography using (0-50%) acetone-hexanes as mobile phase to give the title compound.

LC/MS: [(M+1)]′=189.

### Step B: 5-(1,2-Dihydroxypropyl)-4-methylisobenzofuran-1(3H)-one

To (E)-4-methyl-5-(prop-1-en-1-yl)isobenzofuran-1(3H)-one (300 mg, 1.59 mmol) in acetonitrile/water (10/1, 18 mL) was added NMO (243 mg, 2.07 mmol) and potassium osmate(VI) dihydrate (29.4 mg, 0.080 mmol) at 0° C. The reaction mixture was allowed to warm to rt and stirred at rt for 2 h. TLC showed the reaction completed. The reaction mixture was filtered through a pad of silica gel, rinsed with 10% MeOH/DCM. The crude product was purified with column chromatography (0-10% MeOH/DCM) to give 5-(1,2-dihydroxypropyl)-4-methylisobenzofuran-1(3H)-one. LC/MS: [(M+1)]⁺=223.

### Intermediate 6

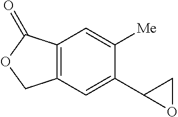 [SMILES: Cc1cc2c(cc1C1CO1)COC2=O]

### Step A: 5-prop-2-en-1-yl-2-benzofuran-1(3H)-one

A mixture of 5-bromo-2-benzofuran-1(3H)-one (15.0 g, 70.4 mmol), allyl-tributyl-stannane (25.6 g, 77.5 mmol), LiCl (11.8 g, 282 mmol) and Pd(PPh₃)₄ (1.2 g, 1.0 mmol) in 100 mL toluene was heated under N₂ between about 90 to about 100° C. overnight. After cooling to rt, the mixture was diluted with 250 mL EtOAc and filtered. The filtrate was washed with water and brine, dried over anhydrous Na₂SO₄ and concentrated to dryness. The residue was purified via column (DCM/PE=1:5) to give title compound.

### Step B: 5-(2-hydroxyethyl)-2-benzofuran-1(3H)-one

To a solution of 5-prop-2-en-1-yl-2-benzofuran-1(3H)-one (13.5 g, 45.2 mmol) in 200 mL DCM/MeOH (V/V=1:1) was bubbled O₃ at −78° C. for 30 min, and N₂ was bubbled for another 15 min at −78° C. Then 20 mL of Me₂S were added, and the mixture was stirred at r.t. overnight before concentrating to dryness. The residue was dissolved in MeOH (100 mL) and then cooled to 0° C. NaBH₄ (5.90 g, 155 mmol) was added in portions. The resulting mixture was stirred at 0° C. for 1 h, then quenched with citric acid (aq.) and extracted three times with EtOAc. The combined organic layers were washed with NaHCO₃ (aq.) and brine, dried over anhydrous Na₂SO₄ and concentrated to dryness. The residue was purified via column chromatography (EtOAc/Petrol Ether=1:5) to give title compound.

¹H-NMR (400 MHz, CDCl₃) δ ppm 7.86 (d, J=7.8 Hz, 1H), 7.41 (d, J=7.8 Hz, 1H), 7.38 (s, 1H), 5.29 (s, 2H), 3.92-3.98 (m, 2H), 3.01 (t, J=6.4 Hz, 2H).

### Step C: 5-(2-hydroxyethyl)-6-iodo-2-benzofuran-1(3H)-one

To a cooled (0° C.) solution of 5-(2-hydroxyethyl)-2-benzofuran-1(3H)-one (9.00 g, 50.6 mmol) in 100 mL of TfOH was added NIS (12.5 g, 55.6 mmol), then the mixture was stirred at 0° C. for 2 hrs and then poured into ice-water (500 mL). The solution was extracted three times with 500 mL of EtOAc and the combined organic layers were washed with saturated NaHCO₃ and brine, dried over anhydrous sodium sulfate, filtered and concentrated. The residue was purified by column chromatography (EtOAc/PE=1:5) to give the desired 5-(2-hydroxyethyl)-6-iodo-2-benzofuran-1(3H)-one and isomeric by-product 5-(2-hydroxyethyl)-4-iodo-2-benzofuran-1(3H)-one. ¹H-NMR (400 MHz, CDCl₃) δ ppm 7.84 (d, J=7.8 Hz, 1H), 7.46 (d, J=7.8 Hz, 1H), 5.09 (s, 2H), 3.93 (q, J=6.3 Hz, 2H), 3.16 (t, J=6.3 Hz, 2H), 1.45 (t, J=5.5 Hz, 1H).

### Step D: 5-(2-hydroxyethyl)-6-methyl-2-benzofuran-1(3H)-one

To a flask charged with 5-(2-hydroxyethyl)-6-iodo-2-benzofuran-1(3H)-one (6.00 g, 19.7 mmol) and a stir bar was added Pd₂(dba)₃ (452 mg, 0.493 mmol), PPh₃ (1 g, 4 mmol) and NMP (50 mL). The mixture was purged with N₂ and heated to 50° C. for 10 min, followed by addition of CuI (375 mg, 1.97 mmol). After the mixture was heated for another 10 min, Sn(CH₃)₄ (5.30 g, 29.6 mmol) was added into the reaction, and it was heated to 120° C. for 2 h. After cooled to room temperature, the mixture was diluted with saturated NH₄Cl (200 mL) and extracted with EtOAc (3 times 200 mL). The combined organic layers were washed with water and brine, dried over anhydrous sodium sulfate, filtered and concentrated. The residue was purified by prep-HPLC to give title compound. ¹H-NMR (400 MHz, CDCl₃) δ ppm 7.72 (s, 1H), 7.33 (s, 1H), 5.27 (s, 2H), 3.93 (t, J=6.3 Hz, 2H), 3.01 (t, J=6.3 Hz, 2H), 2.44 (s, 3H).

### Step E: 2-(6-methyl-1-oxo-1,3-dihydro-2-benzofuran-5-yl)ethyl methanesulfonate

To a solution of 5-(2-hydroxyethyl)-6-methyl-2-benzofuran-1(3H)-one (1.20 g, 6.25 mmol) and TEA (2.5 g, 25 mmol) in DCM (100 mL) was added MsCl (1.40 g, 12.5 mmol) at 0° C. The mixture was stirred at ambient temperature overnight and then was washed with water and brine. The organic layer was dried and concentrated to dryness. The collected title compound was used for the next step without any purification.

### Step F: 5-ethenyl-6-methyl-2-benzofuran-1(3H)-one

To a mixture of 2-(6-methyl-1-oxo-1,3-dihydro-2-benzofuran-5-yl)ethyl methanesulfonate (2.00 g, 7.41 mmol) and TEA (5 mL) in DCM (50 mL) was added DBU (5 mL) slowly at 0° C. The mixture was stirred at rt overnight, and then was diluted with 50 mL of DCM, washed with 2 N HCl in three times and brine. The organic layer was dried and concentrated to dryness. The residue was purified by prep-TLC to give 5-ethenyl-6-methyl-2-benzofuran-1(3H)-one.

### Step G: 6-methyl-5-oxiran-2-yl-2-benzofuran-1(3H)-one

To a solution of 5-ethenyl-6-methyl-2-benzofuran-1(3H)-one (1.00 g, 5.75 mmol) in 50 mL of DCM was slowly added mCPBA (3.50 g, 17.4 mmol) in 50 mL of DCM at 0° C. The mixture was warmed to room temperature, and stirred for 2 days. The mixture was washed with aqueous Na₂SO₃ until KI indicator paper didn't change color. The organic layer was washed with brine and then concentrated. The residue was purified via silica column to give 6-methyl-5-oxiran-2-yl-2-benzofuran-1(3H)-one. LC-MS M+1 (calc. 191. found 191).

### Intermediates 6A and 6B

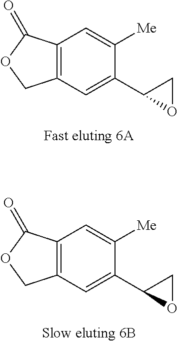 [SMILES: Cc1cc2c(cc1[C@@H]1CO1)COC2=O ; Cc1cc2c(cc1[C@H]1CO1)COC2=O]

### 6A: (R)-6-methyl-5-(oxiran-2-yl)isobenzofuran-1(3H)-one and (S)-6-methyl-5-(oxiran-2-yl)isobenzofuran-1(3H)-one

### 6B: (S)-6-methyl-5-(oxiran-2-yl)isobenzofuran-1(3H)-one and (S)-6-methyl-5-(oxiran-2-yl)isobenzofuran-1(3H)-one

The title compounds were obtained by chiral SFC separation of the racemic 6-methyl-5-oxiran-2-yl-2-benzofuran-1(3H)-one (INTERMEDIATE 6) using a CHIRALPAK® AD column (250 mm×50 mm, 10 um); mobile phase: A: supercritical CO₂, B: MeOH, A:B=85:15 at 250 ml/min. First peak to elute (6A): HNMR 400 MHz CDCl3 δ 7.68 (s, 1H), 7.36 (s, 1H), 5.24 (d, J=3.6 Hz, 2H), 4.05 (dd, J=2.8 Hz, 3.6 Hz, 1H), 3.24 (dd, J=4.0 Hz, 6.4 Hz, 1H), 2.63 (dd, J=2.8 Hz, 6.4 Hz, 1H), 2.50 (s, 3H); second peak to elute (7B): 400 MHz CDCl₃ δ 7.68 (s, 1H), 7.35 (s, 1H), 5.24 (d, J=3.6 Hz, 2H), 4.05 (dd, J=2.8 Hz, 3.6 Hz, 1H), 3.24 (dd, J=4.0 Hz, 6.4 Hz, 1H), 2.63 (dd, J=2.8 Hz, 6.4 Hz, 1H), 2.50 (s, 3H).

### Intermediate 7

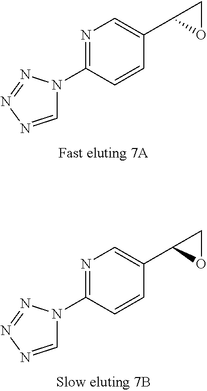 [SMILES: c1cc(-n2cnnn2)ncc1[C@H]1CO1 ; c1cc(-n2cnnn2)ncc1[C@@H]1CO1]

### 7A: (R)-5-(oxiran-2-yl)-2-(1H-tetrazol-1-yl)pyridine

### 7B: (S)-5-(oxiran-2-yl)-2-(1H-tetrazol-1-yl)pyridine

### Step A: 5-Bromo-2-(1H-tetrazol-1-yl)pyridine

To a solution of 5-bromopyridin-2-amine (5.0 g, 28.9 mmol) in acetic acid (40 ml, 699 mmol) was added (diethoxymethoxy) ethane (7.70 ml, 46.2 mmol), followed by sodium azide (2.82 g, 43.3 mmol). The mixture was heated at 80° C. for 1 h, cooled to room temperature and diluted with water. Precipitate was collected by filtration and dried under high vacuum to provide the title compound.

### Step B: 5-Ethenyl-2-(1H-tetrazol-1-yl)pyridine

To a stirring solution of 5-bromo-2-(1H-tetrazol-1-yl)pyridine (1.0 g, 4.42 mmol), in EtOH (70 mL) were added bis[(diphenylphosphino)ferrocene]dichloropalladium(II), complex with dichloromethane (0.361 g, 0.442 mmol), potassium vinyl trifluoroborate (1.18 g, 8.85 mmol, 2 equiv.), triethylamine (1.23 mL, 8.85 mmol, 2 equiv), and water (0.5 mL). The reaction mixture was heated at reflux (90° C., oil bath) under N₂. Upon completion (1-2 h) as determined by reverse phase HPLC-MS and TLC (eluent: 10% ethyl acetate in hexane), the mixture was cooled to room temperature, diluted with water. The organic layer was separated, and the aqueous was extracted with EtOAc. The combined organic layers were washed with brine, dried over MgSO₄, and concentrated. The crude material was chromatographed over a column of SiO₂ (0-20% EtOAc in hexane as eluent). Evaporation of the solvent yielded the title compound. LCMS [M+1]⁺=174.03.

### Step C: 5-(Oxiran-2-yl)-2-(1H-tetrazol-1-yl)pyridine

To a solution of 5-ethenyl-2-(1H-tetrazol-1-yl)pyridine (0.664 g, 3.83 mmol) in a 2:1 ratio of H₂O:t-BuOH (30 mL) was added N-bromosuccinimide (0.751 g, 4.22 mmol) in portions over 5 min. The mixture was heated at 40° C. for 1 h, cooled to 5° C., made basic with sodium hydroxide aqueous solution (0.46 g in 5 mL of H₂O, 11.50 mmol), stirred for another 1 h at the same temperature, and poured into H₂O (10 mL). The product was precipitated out as white solid. The solid was collected by filtration, washed with water, and dried in vacuum. ¹H NMR (500 MHz, DMSO-d₆), δ 10.17 (s, 1H), 8.60 (d, J=1.4 Hz, 1H), 8.04-7.99 (m, 2H), 4.14 (dd, J=2.7 Hz, J=2.8 Hz, 1H), 3.23 (t, J=4.6 Hz, 1H), 3.02 (dd, J=25 Hz, 1H); LCMS [M+1]⁺=190. Further chiral separation (AD-H 30×250 mm, 50% MeOH/CO₂, 70 mL/min, 100 bar, 46 mg in MeOH/DCM) conducted by the separation and purification group afforded fast eluted 7A (R)-5-(oxiran-2-yl)-2-1H-tetrazol-1-yl)pyridine and slow eluted 7B (S)-5-(oxiran-2-yl)-2-(1H-tetrazol-1-yl)pyridine. Absolute chemistry was determined by using vibrational VCD spectroscopy with high confidence. Analysis was done comparing experimental data to the calculated VCD and IR spectra of the (R) and (S) compounds.

### Intermediate 8

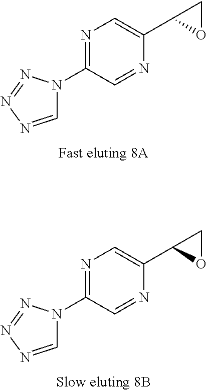 [SMILES: c1nc(-n2cnnn2)cnc1[C@H]1CO1 ; c1nc(-n2cnnn2)cnc1[C@@H]1CO1]

### 8A: (R)-2-(oxiran-2-yl)-5-(1H-tetrazol-1-yl)pyrazine

### 8B: (S)-2-(oxiran-2-yl)-5-(1H-tetrazol-1-yl)pyrazine

### Step A: 2-Bromo-5-(1H-tetrazol-1-yl)pyrazine

To a solution of 5-bromopyrazin-2-amine (10.75 g, 57.5 mmol) in ethyl acetate (150 ml) was added trimethylsilyl 2,2,2-trifluoroacetate (16.88 ml, 98 mmol). After the mixture was stirred for 5 min, triethoxymethane (17.21 ml, 103 mmol) was added. The resulting mixture was stirred for another five min, and this was followed by addition of zidotrimethylsilane (12.09 ml, 92 mmol). Stirring continued at rt for 2 days, and the mixture was concentrated under reduced pressure. Recrystallization of the residue from ethyl acetate afforded 2-bromo-5-(1H-tetrazol-1-yl)pyrazine. LCMS [M+2+1]⁺=228.98.

### Step B: 2-(1H-tetrazol-1-yl)-5-vinylpyrazine

A solution of 2-bromo-5-(1H-tetrazol-1-yl)pyrazine (11.2 g, 49.3 mmol), potassium vinyltrifluoroborate (13.22 g, 99.0 mmol), 1,1′-bis(diphenylphosphino)ferrocene-palladium(ii)dichloride dichloromethane complex (2.01 g, 2.47 mmol), and TEA (13.75 ml, 99.0 mmol) in ethanol (150 ml) was heated at reflux at 82° C. for 4 h. The reaction mixture was cooled to rt, and the precipitation was filtered off. The filtrate was concentrated, and the residue was purified by flash chromatography (Biotage® Si, ethyl acetate in hexane: 35 to 45%) affording 2-(1H-tetrazol-1-yl)-5-vinylpyrazine LCMS [M+1]⁺=175.10. The filter cake was stirred in DCM (50 mL), and the solid was filtered off. The filtrate was concentrated to afford more 2-(1H-tetrazol-1-yl)-5-vinylpyrazine.

### Step C: 2-(oxiran-2-yl)-5-(1H-tetrazol-1-yl)pyrazine

To a suspension of 2-(1H-tetrazol-1-yl)-5-vinylpyrazine (6.7 g, 38.5 mmol) in t-BuOH:water (96 ml: 190 ml) was added N-bromosuccinimide (7.53 g, 42.3 mmol) in portion at RT. The mixture was heated at 50° C. for 1 h, and cooled to 0° C. in an ice bath. NaOH (4.61 g in 30 mL water, 115 mmol) was added dropwise, and the resulting mixture was stirred at the same temperature for 20 min. The white solid product was collected by filtration, washed with water, dried under vacuum to give 2-(1H-tetrazol-1-yl)-5-vinylpyrazine LCMS [M+1]⁺=191.07. Chiral separation (AD-H 30×250 mm, 50% MeOH/CO₂, 70 mL/min, 100 bar, MeOH/DCM) conducted by the separation and purification group afforded fast eluted isomer 8A (S)-2-(oxiran-2-yl)-5-(1H-tetrazol-1-yl)pyrazine and slow eluted isomer 8B (R)-2-(oxiran-2-yl)-5-(1H-tetrazol-1-yl)pyrazine. LCMS [M+1]⁺=191.10.

### Intermediate 9

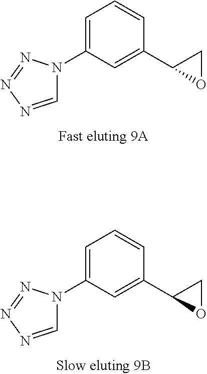 [SMILES: c1cc([C@@H]2CO2)cc(-n2cnnn2)c1 ; c1cc([C@H]2CO2)cc(-n2cnnn2)c1]

### 9A: (R)-1-(3-(oxiran-2-yl)phenyl)-1H-tetrazole

### 9B: (S)-1-(3-(oxiran-2-yl)phenyl)-1H-tetrazole

### Step A: 1-(3-bromophenyl)-1H-tetrazole

A 10-20 mL Biotage Initiator™ microwave reactor vessels equipped with magnetic stir bars and septum cap was charged with 3-bromoaniline (0.633 mL, 5.81 mmol), AcOH (10 mL), triethyl orthoformate (2.90 mL, 17.44 mmol) and NaN₃ (1.134 g, 17.44 mmol). The reaction mixtures were placed into a pre-heated 80° C. metal reaction block and stirred for 3 h. LCMS indicates complete conversion to the desired tetrazole. The reaction mixture was cooled to room temperature and diluted with deonized H₂O. The aqueous medium was extracted with EtOAc, the organic layer was separated and washed with saturated aq. NaCl—dried (Na₂SO₄), filtered, concentrated under reduced pressure and dried in vacuo to afford the title compound. LC/MS: [(M+2)]⁺=227.

### Step B: 1-(3-vinylphenyl)-1H-tetrazole

To a flask was charged 1-(3-bromophenyl)-1H-tetrazole (640 mg, 2.84 mmol), potassium vinyltrifluoroborate (571 mg, 4.27 mmol), PdCl2(dppf) (104 mg, 0.142 mmol). The flask was sealed, degassed, and filled with EtOH (14 mL) and Et₃N (1.19 mL, 8.53 mmol). The reaction mixture was heated at 85° C. for 3 h, and filtered to give the crude product, which was purified by column chromatography (0-100% EtOAc/hexanes) to give the title compound. LC/MS: [(M+1)]⁺=173.

### Step C: (S)-1-(3-(oxiran-2-yl)phenyl)-1H-tetrazole and (R)-1-(3-(oxiran-2-yl)phenyl)-1H-tetrazole

To 1-(3-vinylphenyl)-1H-tetrazole (444 mg, 2.58 mmol) in DCM (25 mL) was added mCPBA (1335 mg, 7.74 mmol). The reaction mixture was stirred at rt overnight, and washed with NaHCO₃, brine, dried and evaporated to give the crude product, which was purified by column chromatography (0-100% EtOAc/hexanes) to afford the racemic compound. The racemate was submitted for SFC chiral separation with method CHIRALPAC® IC, 20% (2:1 MeOH:MeCN)/CO₂ to give the Fast and Slow eluting enantiomers. ¹H NMR (500 MHz, CDCl₃) δ 9.05 (s, 1H), 7.70-7.50 (m, 3H), 4.01 (dd, J=4.0, 2.6 Hz, 1H), 3.25 (dd, J=5.5, 4.0 Hz, 1H), 2.85 (dd, J=5.5, 2.6 Hz, 1H).

The following epoxides in Table 1 were prepared according to the method described for INTERMEDIATES 7 to 9.

TABLE 1

| | Starting | | | LC-MS |
|---|---|---|---|---|
| | material | | | [M + 1]⁺ or |
| INT. | and method | Structure A | Structure B | [M + 1-28]⁺ |
| 10 | 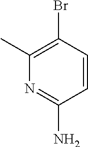 | 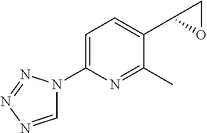 | 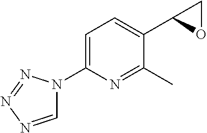 | 188.1 |
| | Method for | Fast eluted 10A | Slow eluted 10B | |
| | Int. 7 | (R)-2-methyl-3-(oxiran-2-yl)- | (S)-2-methyl-3-(oxiran-2-yl)- | |
| | | 6-(1H-tetrazol-1-yl)pyridine | 6-(1H-tetrazol-1-yl)pyridine | |
| 11 | 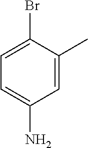 | 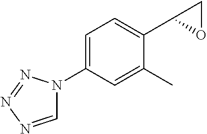 | 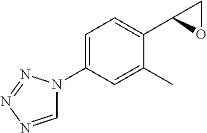 | 203.1 |
| | Method for | Fast eluted 11A | Slow eluted 11B | |
| | Int. 8 | (R)-1-(3-methyl-4-(oxiran-2- | (S)-1-(3-methyl-4-(oxiran-2- | |
| | | yl)phenyl)-1H-tetrazole | yl)phenyl)-1H-tetrazole | |

### Intermediate 12

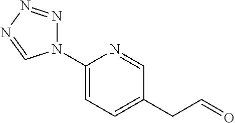 [SMILES: O=CCc1ccc(-n2cnnn2)nc1]

### Step A: 2-(6-(1H-Tetrazol-1-yl)pyridin-3-yl)ethanol

To 5-(oxiran-2-yl)-2-(1H-tetrazol-1-yl)pyridine (INTERMEDIATE 7, 500 mg, 2.64 mmol) in ethanol (5.3 mL) was added 10% Pd/C (101 mg, 0.952 mmol) and HCOONH₄ (500 mg, 7.93 mmol). The reaction mixture was vigorously stirred for 1.5 h, and filtered through a pad of silica gel. The filtrate was evaporated to give 2-(6-(1H-tetrazol-1-yl)pyridin-3-yl)ethanol. ¹H NMR (500 MHz, CDCl₃) δ 9.54 (s, 1H), 8.43 (d, J=2.0 Hz, 1H), 8.02 (d, J=8.3 Hz, 1H), 7.90 (dd, J=8.3, 2.0 Hz, 1H), 3.91 (t, J=6.3 Hz, 2H), 2.96 (t, J=6.3 Hz, 2H).

### Step B: 2-(6-(1H-Tetrazol-1-yl)pyridin-3-yl)acetaldehyde

To 2-(6-(1H-tetrazol-1-yl)pyridin-3-yl)ethanol (100 mg, 0.523 mmol) in DCM (2.6 mL) was added Dess-Martin periodinane (333 mg, 0.785 mmol). The mixture was vigorously stirred for 1.5 h, and diluted with 10% Na₂S₂O₂, NaHCO₃, and stirred for 20 min. The aqueous layer was extracted with DCM, and the organic layers were washed with brine, dried, and concentrated to give 2-(6-(1H-tetrazol-1-yl)pyridin-3-yl)acetaldehyde. LC/MS: [(M+1)]⁺=190.

### Intermediate 13

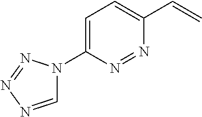

### Step A: 2-Chloro-6-(1H-tetrazol-1-yl)pyridazine

To a solution of 6-chloropyridazin-3-amine (10.0 g, 77.0 mmol) in ethyl acetate (200 mL) was added trimethylsilyl 2,2,2-trifluoroacetate (22.7 mL, 131 mmol). The mixture was stirred for 5 min, and triethoxymethane (22.8 ml, 137 mmol) was added. After the resulting mixture was stirred for another 5 min, azidotrimethylsilane (16.2 ml, 124 mmol) was added. Stirring continued at rt for 2 days, and the reaction mixture was filtered, and the solid was rinsed with ethyl acetate to afford the title compound. LCMS [M+1]⁺=183.

### Step B: 3-(1H-tetrazol-1-yl)-6-vinylpyridazine

A solution of 2-chloro-6-(1H-tetrazol-1-yl)pyridazine (6.0 g, 32.9 mmol), potassium vinyltrifluoroborate (6.6 g, 49.3 mmol), 1,1′-bis(di-tert-butylphosphino)ferrocene-palladium dichloride (2.14 g, 3.29 mmol), and potassium phosphate aqueous solution (32.9 mL, 2 M, 65.7 mmol) in THF (160 ml) was heated at 80° C. overnight. The reaction mixture was allowed to cool to rt and separated. The aqueous phase was extracted with EtOAc, and the organic layers were dried over MgSO₄, and concentrated, and the residue was purified by silica gel chromatography (EtOAc in hexane: 0 to 100%) to afford the title compound. LCMS [M+1-28]⁺=147.

### Intermediate 14

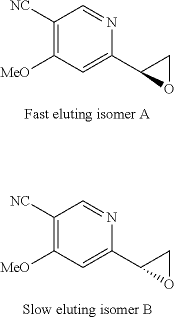 [SMILES: COc1cc([C@H]2CO2)ncc1C#N ; COc1cc([C@@H]2CO2)ncc1C#N]

### 14A: (S)-4-Methoxy-6-(oxiran-2-yl)pyridine-3-carbonitrile

### 14B: (R)-4-Methoxy-6-(oxiran-2-yl)pyridine-3-carbonitrile

### Step A: 5-Bromo-2-chloro-4-methoxypyridine

To a solution of 2-chloro-4-methoxypyridine (10.0 g, 69.7 mmol) in 50 mL of sulfuric acid at 0° C. was added NBS. The reaction mixture was allowed to stir and warm up to room temperature for 2 hour and then heated at 60° C. for 5 h. Then it was cooled to room temperature and neutralized with 1 N NaOH (pH˜7), diluted with water (50 mL) and the aqueous layer was extracted with ethyl acetate (2×100 mL). The organic layers were washed with water (2×50 mL), sat. NaHCO₃, brine, dried over Mg₂SO₄ and concentrated to provide an oil, which was chromatographed. On elution with 0-25% EtOAc/hexanes the final product was obtained. ¹H NMR (500 MHz, DMSO-d₆), δ 8.4 (s, 1H), 7.29 (s, 1H), 3.97 (s, 3H); LC/MS (M+1)¹=223.

### Step B: 6-Chloro-4-methoxypyridine-3-carbonitrile

A solution of 5-bromo-2-chloro-4-methoxypyridine (5.0 g, 22.48 mmol) in DMF (80 mL) was purged with nitrogen for 15 min. At this point, Zn(CN)₂ (3.96 g, 33.7 mmol) and Pd(Ph₃P)₄ (2.60 g, 2.25 mmol) were added, successively. The resulting suspension was stirred at 95° C. for 12 h under nitrogen atm. The reaction mixture was cooled to ambient temperature, filtered to remove inorganic solid. The solvent (DMF) was evaporated to provide the crude residue as an oil, which was purified on silica gel and eluted with 0-30% ethyl acetate/hexanes to afford the product. ¹H NMR (500 MHz, DMSO-d₆), δ 8.69 (s, 1H), 7.50 (s, 1H), 4.04 (s, 3H); LC/MS (M+1)¹=169.

### Step C: 6-Ethenyl-4-methoxypyridine-3-carbonitrile

A 20 mL microwave tube was charged with 6-chloro-4-methoxypyridine-3-carbonitrile (200.0 mg, 1.2 mmol), bis(diphenylphosphino)ferrocene]dichloropalladium(II), complex with dichloromethane (97.0 mg, 0.12 mmol), potassium vinyl trifluoroborate (318.0 mg, 2.37 mmol), and triethylamine (0.33 mL, 2.37 mmol), and EtOH (6 mL). The microwave tube was evacuated and filled with nitrogen (two times) and heated to 140° C. After 1 h, the reaction mixture was diluted with water and extracted with EtOAc. The combined organic layers were washed with brine and dried over Na₂SO₄. The extracts were concentrated and chromatographed over a column of SiO₂ (0-30% EtOAc/hexanes as eluent). Evaporation of the solvent yielded the title compound.

¹H NMR (500 MHz, DMSO-d₆), δ 8.65 (s, 1H), 6.89 (s, 1H), 6.83 (dd, J=10.7 Hz, 1H), 6.42 (d, J=7.3 Hz, 1H), 5.70 (d, J=10.6 Hz, 1H) 4.05 (s, 3H); LC/MS (M+1)⁺=161.

### Step D: 6-(2-Bromo-1-hydroxyethyl)-4-methoxypyridine-3-carbonitrile

A solution of 6-ethenyl-4-methoxypyridine-3-carbonitrile (80.0 mg, 0.499 mmol) in 1, 4-dioxane (8 mL) and H₂O (4 mL) was treated with N-bromosuccinimide (89.0 mg, 0.499 mmol, 1.0 equiv). The reaction mixture was allowed to stir at room temperature overnight. The reaction mixture was poured into H₂O (8 mL) and extracted with EtOAc (3×30 mL). The combined organic layers were washed with saturated aqueous NaCl (1×30 mL), dried over Na₂SO₄. Evaporation of the solvent gave an oil that was purified over SiO₂ (0-30% EtOAc/hexanes as eluent) yielding 6-(2-bromo-1-hydroxyethyl)-4-methoxypyridine-3-carbonitrile. ¹H NMR (500 MHz, DMSO-d₆), δ 8.65 (s, 1H), 7.19 (s, 1H), 5.05 (t, J=5.4 Hz, 1H), 4.05 (s, 3H), 3.85 (dd, J=4.5 Hz, 1H), 3.75 (dd, J=6.1 Hz, 1H); LC/MS (M+1)⁺=241.

### Step E: 14B: (R)-4-methoxy-6-(oxiran-2-yl)pyridine-3-carbonitrile and 14A: (S)-4-methoxy-6-(oxiran-2-yl)pyridine-3-carbonitrile

A solution of 6-(2-bromo-1-hydroxyethyl)-4-methoxypyridine-3-carbonitrile (74.0 mg, 0.288 mmol) in anhydrous methanol (7 mL) was treated with sodium carbonate (61.0 mg, 0.576 mmol, 2.0 equiv), and allowed to stir at room temperature overnight. The solvent was evaporated. The residue was taken up in EtOAc (30 mL) and washed with water and brine. After drying over Na₂SO₄, the organic layer was removed and the residue was purified over SiO₂ (10-45% EtOAc/hexanes as eluent) to yield 4-methoxy-6-(oxiran-2-yl)pyridine-3-carbonitrile. ¹H NMR (500 MHz, DMSO-d₆), δ 8.64 (s, 1H), 6.87 (s, 1H), 4.08 (dd, J=2.6 Hz, J=2.3 Hz, 1H), 4.03 (s, 3H), 3.26 (dd, J=4.6 Hz, J=5.4 Hz, 1H), 2.87 (dd, J=2.2 Hz, J=2.4 Hz, 1H); LC/MS (M+1)⁺=177.

Resolution of the epoxides was carried out (preparative SFC, 160 mL/min., 10% MeOH in SC CO₂, AD-H) to provide:

Fast Eluting Isomer 14A: (M+1)⁺=177.

Slow Eluting Isomer 14B: (M+1)⁺=177.

Absolute chemistry was determined by using VCD spectroscopy with high confidence. Analysis was done comparing experimental data to the calculated VCD and IR spectra of the (R) and (S) compounds.

### Intermediate 15

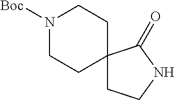 [SMILES: CC(C)(C)OC(=O)N1CCC2(CCNC2=O)CC1]

tert-Butyl 1-oxo-2,8-diazaspiro[4.5]decane-8-carboxylate is commercially available from a number of vendors, for example, Shanghai AQ BioPharma Co., Ltd, catalog #ABP1882. Alternatively, it may be prepared in various ways, including the procedure described below:

### Step A: 1-tert-Butyl 4-methyl 4-(cyanomethyl)piperidine-1,4-dicarboxylate

To a solution of commercially available 1-tert-butyl 4-methyl piperidine-1,4-dicarboxylate (200 g, 0.82 mol) in anhydrous THF (2 L) was added LDA (2M in THF, 575 mL, 1.15 mol) drop-wise at −65° C. under N₂. The mixture was stirred at −65° C. for 1.5 h. To the mixture was added bromoacetonitrile (148 g, 1.23 mol) in anhydrous THF (500 mL) at −65° C. The mixture was stirred at −65° C. for 1 h, then warmed up to room temperature and stirred overnight. The reaction was quenched with water (800 mL) at 0° C. and the combined reaction mixture was concentrated in vacuum to give a crude product, which was extracted with ethyl acetate (1 L×3). The combined organic phases were washed with brine (1 L) and dried over Na₂SO₄. The organic layer was filtered and the filtrate was concentrated in vacuum to give a crude product, which was purified by column chromatography on silica gel eluting with petroleum ether/ethyl acetate (from petroleum ether to 2/1) to give title compound. ¹H-NMR (400 MHz, CDCl₃) δ 3.900-3.750 (m, 5H), 3.120-3.000 (m, 2H), 2.612-2.562 (m, 2H), 2.190-2.111 (m, 2H), 1.590-1.502 (m, 2H), 1.402 (s, 9H).

### Step B: tert-Butyl 1-oxo-2,8-diazaspiro[4.5]decane-8-carboxylate

A suspension of 1-tert-butyl 4-methyl 4-(cyanomethyl)piperidine-1,4-dicarboxylate (70.0 g, 247.9 mmol) and Raney Ni (60 g) in MeOH (1500 mL) and NH₃.H₂O (80 mL) was stirred at 2 MPa of hydrogen at 50° C. for 18 h. The reaction mixture was filtered through a pad of CELITE® and the filtrate was concentrated under vacuum to give a crude product, which was washed with ethyl acetate (200 mL) to give title compound. ¹H-NMR (400 MHz, CDCl₃) δ 6.05 (s, 1H), 4.0 (s, 2H), 3.37-3.34 (m, 2H), 3.02-2.96 (m, 2H), 2.08-2.05 (m, 2H), 1.88-1.87 (m, 2H), 1.51-1.41 (m, 11H).

### Intermediate 16

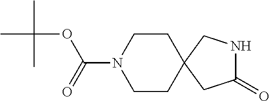 [SMILES: CC(C)(C)OC(=O)N1CCC2(CC1)CNC(=O)C2]

### Step A: tert-butyl 4-(2-ethoxy-2-oxoethylidene)piperidine-1-carboxylate

Into a 10-L 4-necked round-bottom flask purged and maintained with an inert atmosphere of nitrogen was placed a suspension of NaH (74.0 g, 2.16 mol 1.05 equiv, 70%) in tetrahydrofuran (2000 mL) at 0° C., then added dropwise ethyl 2-(diethoxyphosphoryl)acetate (514 g, 2.06 mol, 1.05 equiv, 98%) with stirring at 0° C. This was followed by the addition of a solution of tert-butyl 4-oxopiperidine-1-carboxylate (400 g, 1.97 mol, 1.00 equiv, 98%) in tetrahydrofuran (1200 mL) dropwise with stirring at 0° C. The resulting solution was stirred for 60 min at room temperature and then quenched by the addition of 2000 mL of water. The resulting solution was extracted with 2×1000 mL of ethyl acetate. The organic layers were combined, dried over anhydrous magnesium sulfate and concentrated under vacuum. The residue was washed with 1×1000 mL of hexane and dried. This resulted in tert-butyl 4-(2-ethoxy-2-oxoethylidene)piperidine-1-carboxylate.

### Step B: tert-butyl 4-(2-ethoxy-2-oxoethyl)-4-(nitromethyl)piperidine-1-carboxylate

Into a 3000-mL 4-necked round-bottom flask were potassium carbonate (93.2 g, 662 mmol, 0.50 equiv) and DMSO (2000 mL). The resulting solution was heated to 80° C. This was followed by the addition of tert-butyl 4-(2-ethoxy-2-oxoethylidene)piperidine-1-carboxylate (368 g, 1.30 mol, 1.00 equiv, 95%) and CH₃ NO₂ (417 g, 6.70 mol, 5.00 equiv, 98%) slowly. The resulting solution was stirred for 120 min at 90° C. After cooled to room temperature, the reaction mixture was adjusted to Ph 5 with HCl (0.5 mol/L) and diluted with 2000 mL of water. The resulting solution was extracted with 3×1500 mL of ether. The organic layers were combined, washed with 1×2000 mL of water and 1×2000 mL of saturated brine, dried and concentrated under vacuum. The residue was applied onto a silica gel column and eluted with ethyl acetate/petroleum ether (1:20-1:15-1:10) to afford the title compound.

### Step C: 3-oxo-2,8-diaza-spiro[4,5]decane-8-carboxylic acid tert-butylester

A mixture of tert-butyl 4-(2-ethoxy-2-oxoethyl)-4-(nitromethyl)piperidine-1-carboxylate (330 g, 990 mmol, 1.00 equiv, 99%) and Ni (40 g, 0.15 equiv) in ethanol (1200 mL) was stirred for 24 h under a hydrogen atmosphere at room temperature. The solid was filtered out. The filtrate was concentrated under vacuum. The crude product was purified by re-crystallization from ether to afford the title compound. LC-MS (ES, m/z): 199 [M+H]⁺; H-NMR (400 MHz, CDCl3, ppm): 1.447-1.476 (9H, s), 1.597-1.673 (4H, m, J=30.4 Hz), 2.235 (2H, s), 3.226 (2H, s), 3.284-3.348 (2H, m, J=25.6 Hz), 3.507-3.567 (2H, m, J=24 Hz), 6.048 (1H, s).

### Intermediate 17

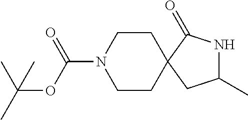 [SMILES: CC1CC2(CCN(C(=O)OC(C)(C)C)CC2)C(=O)N1]

### Step A: 1-tert-Butyl 4-methyl 4-(2-methylallyl)piperidine-1,4-dicarboxylate

A solution of N-Boc-piperidine-4-carboxylic acid methyl ester (2.00 g, 8.22 mmol) in THF (40 mL) was cooled to −78° C. Under nitrogen, a 2.0 M THF solution of LDA (6.17 mL, 12.3 mmol) was added dropwise. The reaction mixture was stirred at −78° C. for 30 minutes before a solution of 3-bromo-2-methylpropene (1.60 g, 11.9 mmol) in THF (2 mL) was added. After the reaction was stirred for 1 hour at this temperature, a sample was taken for LC-MS analysis and it showed that the reaction was completed. The reaction was quenched by adding saturated ammonium chloride solution (5 mL) and the mixture was allowed to warm up to room temperature. The mixture was then extracted with EtOAc (50 mL twice). The combined organic layers were washed with brine (30 mL), dried over anhydrous sodium sulfate and filtered. The filtrates were concentrated and the crude product was purified by column chromatography eluting with 0-30% ethyl acetate/hexane to give title compound. LC-MS (IE, m/z): 242.21 [M−56+1]⁺.

### Step B: 1-tert-Butyl 4-methyl 4-(2-oxopropyl)piperidine-1,4-dicarboxylate

To a solution of 1-tert-butyl 4-methyl 4-(2-methylallyl)piperidine-1,4-dicarboxylate (2.2 g, 7.4 mmol) in dioxane/water (60 mL, 1/1) under nitrogen was added osmium tetroxide (0.038 g, 0.15 mmol) and sodium periodate (2.88 g, 13.5 mmol). The mixture was stirred at room temperature for 3 hours. The mixture was then diluted with dichloromethane (50 mL), washed with 20% Na₂S₂O₃ (20 mL). The organic layers were combined and washed with brine (20 mL), dried over anhydrous sodium sulfate, and filtered. The filtrates were concentrated and the residue was purified by column chromatography eluting with 0-60% ethyl acetate/hexane to afford 1-tert-butyl 4-methyl 4-(2-oxopropyl)piperidine-1,4-dicarboxylate. LC-MS (IE, m/z): 322.26 (M+23)⁺.

### Step C: tert-Butyl 3-methyl-1-oxo-2,8-diazaspiro[4.5]decane-8-carboxylate

1-tert-Butyl 4-methyl 4-(2-oxopropyl)piperidine-1,4-dicarboxylate (1.15 g, 3.84 mmol) in methanol (25 mL) was treated with ammonium acetate (3.85 g, 49.9 mmol), sodium cyanoborohydride (0.681 g, 10.83 mmol) and magnesium sulfate (2.54 g, 21.1 mmol). The mixture was heated at 80° C. in a sealed tube for 12 hours. The reaction mixture was filtered through a pad of CELITE® and the filter cake was washed with methanol. The filtrates were then concentrated and the residue was purified by column chromatography eluting with 0-10% methanol/ethyl acetate to afford the title compound. LC-MS (IE, m/z): 291.27 (M+23)⁺.

### Intermediate 17A and 17B

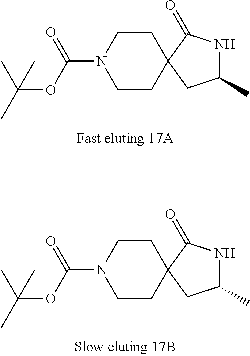

### Fast eluting 17A: (S)-tert-Butyl 3-methyl-1-oxo-2,8-diazaspiro[4.5]decane-8-carboxylate; (R)-tert-Butyl 3-methyl-1-oxo-2,8-diazaspiro[4.5]decane-8-carboxylate

### Slow eluting 17B: (R)-tert-Butyl 3-methyl-1-oxo-2,8-diazaspiro[4.5]decane-8-carboxylate; (R)-tert-Butyl 3-methyl-1-oxo-2,8-diazaspiro[4.5]decane-8-carboxylate

tert-Butyl 3-methyl-1-oxo-2,8-diazaspiro[4.5]decane-8-carboxylate was subjected to SFC purification. The two enantiomers were resolved on CHIRALCEL® IA column eluting with 30% MeOH:MeCN (2:1)/CO₂ (100 bar, 35° C.). The faster eluting isomer was determined to be (S)-tert-Butyl 3-methyl-1-oxo-2,8-diazaspiro[4.5]decane-8-carboxylate and the slower eluting isomer was (R)-tert-Butyl 3-methyl-1-oxo-2,8-diazaspiro[4.5]decane-8-carboxylate based on VCD spectroscopy analysis. LC-MS (IE, m/z): 291 (M+23)⁺.

### Intermediate 18

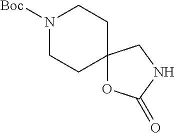 [SMILES: CC(C)(C)OC(=O)N1CCC2(CC1)CNC(=O)O2]

### Step A: tert-butyl 4-(2-ethoxy-2-oxoethyl)-4-hydroxypiperidine-1-carboxylate

To a solution of lithium bis(trimethylsilyl)amide (120 mL, 1.0 M solution in THF, 0.12 mol) in THF (120 mL) at −78° C. was added ethyl acetate (13 mL); then, a solution of tert-butyl 4-oxopiperidine-1-carboxylate (20 g, 0.1 mol) in THF (80 mL) was added at −78° C. After the addition, the mixture was warmed up to 0° C. and stirred for another 2 h. The aqueous layer was extracted with ethyl acetate; the organic phase was washed with brine, dried over Na₂SO₄ and concentrated to afford the crude title compound.

### Step B: 2-(1-(tert-butoxycarbonyl)-4-hydroxypiperidin-4-yl)acetic acid

A solution of tert-butyl 4-(2-ethoxy-2-oxoethyl)-4-hydroxypiperidine-1-carboxylate (30.0 g, 0.105 mol) in methanol (130 mL) and 2N NaOH solution (100 mL, 0.2 mol) was stirred at 25° C. for 1.5 h, then the mixture was evaporated and the aqueous layer was extracted with ethyl acetate. The water phase was adjusted to pH 6 with 2N HCl, the aqueous layer was extracted with ethyl acetate and then the organic phase was washed with brine, dried over Na₂SO₄ and concentrated to afford the crude title compound.

### Step C: tert-butyl 2-oxo-1-oxa-3,8-diazaspiro[4.5]decane-8-carboxylate

A mixture of 2-(1-(tert-butoxycarbonyl)-4-hydroxypiperidin-4-yl)acetic acid (22 g, 0.085 mol), DPPA (30 g, 0.11 mol), Et₃N (150 mL) in Toluene (400 mL) was stirred at 105° C. under nitrogen for 12 h. The reaction mixture was quenched by the addition of the saturated aqueous NaHCO₃, the organic phase was washed with brine, dried over Na₂SO₄, the mixture was concentrated to remove most of toluene, then ether was added and filtered. The filter cake was washed with ether and the solid was dried under vacuum to afford the pure title compound. ¹H NMR (300 MHz, CDCl₃) δ: 5.35 (brs, 1H), 3.83-3.85 (m, 2H), 3.26-3.35 (m, 4H), 1.93-1.97 (m, 2H), 1.68-1.75 (m, 2H), 1.46 (s, 9H).

### Intermediate 19

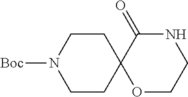 [SMILES: CC(C)(C)OC(=O)N1CCC2(CC1)OCCNC2=O]

### Step A: 1-tert-butyl 4-methyl 4-(allyloxy)piperidine-1,4-dicarboxylate

NaH (0.92 g, 15.4 mol, 60% dispersion in mineral oil) was added the five portions to a stirred solution of compound 1-tert-butyl 4-methyl 4-hydroxypiperidine-1,4-dicarboxylate (2 g, 7.7 mmol) being cooled to 0° C. in DMF (20 mL). After the mixture was stirred at 0° C., the 3-allyl bromide (1.2 g, 10 mmol) was added, dropwise. The mixture was stirred at rt for 16 h. The reaction mixture was quenched by the addition of the saturated aqueous NH₄Cl and evaporated to afford the crude product. The crude product was purified by column chromatography on silica gel eluted with (PE/EA 50:1→30:1→15:1) to give the title compound.

### Step B: 1-tert-butyl 4-methyl 4-(2-oxoethoxy)piperidine-1,4-dicarboxylate

To a solution of 1-tert-butyl 4-methyl 4-(allyloxy)piperidine-1,4-dicarboxylate (1.2 g, 4 mmol) in MeOH (30 mL) was added osmium tetroxide (30 uL, 0.006 mmol, 0.81 g/mL H₂O) and sodium periodate (16 ml, 16 mmol, 1M). The mixture was allowed to stir at rt for 16 hours. The mixture was quenched with Na₂S₂O₃ (50 mg), extracted with ethyl acetate (20 mL×3), dried over Na₂SO₄ and concentrated to afford the crude product, which was further purified by column chromatography on silica gel eluted with (PE/EA 20:1→10:1→5:1→1:1) to give the title compound.

### Step C: 1-tert-butyl 4-methyl 4-(2-(dibenzylamino)ethoxy)piperidine-1,4-dicarboxylate

To a stirred solution of 1-tert-butyl 4-methyl 4-(2-oxoethoxy)piperidine-1,4-dicarboxylate (0.3 g, 1 mmol) in DCE (5 mL) was added dibenzyl amine (0.3 g, 1.5 mmol), the resulted mixture was stirred at room temperature for 1 h. Then sodium triacetoxyborohydride (0.42 g, 2 mmol) was added to the reaction mixture, the reaction mixture was stirred for further 4 h at room temperature. The mixture was quenched with water (5 mL), extracted with DCM (5 mL×3), the combined organic portions were concentrated and purified by column chromatography gel eluted with (PE/EA 5:1→2:1→1:1) to give the title compound.

### Step D: tert-butyl 5-oxo-1-oxa-4,9-diazaspiro[5.5]undecane-9-carboxylate

A mixture of 1-tert-butyl 4-methyl 4-(2-(dibenzylamino)ethoxy)piperidine-1,4-dicarboxylate (290 mg, 0.6 mmol) and 10% palladium hydroxide on carbon (20%, w/w, 30 mg) in MeOH (10 mL) was hydrogenated under 40 psi of hydrogen at 30° C. overnight. Then the mixture was cooled to room temperature and the catalyst was filtered off. The filtrate was concentrated in vacuo to give title compound.

### Intermediate 20

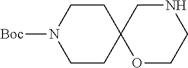 [SMILES: CC(C)(C)OC(=O)N1CCC2(CC1)CNCCO2]

### Step A: tert-Butyl 4-oxopiperidine-1-carboxylate

To a solution of piperidin-4-one (1.0 mol, 100.0 g) and NaHCO₃ (1.6 mmol, 100 g) in H₂O (1000 mL) was added (BOC)₂O (1.2 mol, 191.6 g), the reaction was stirred at 50° C. overnight. The residue was extracted with EtOAc (3×400 mL) and the combined organic layers were washed with brine, dried over Na₂SO₄, filtered and concentrated to give tert-butyl 4-oxopiperidine-1-carboxylate. ¹H NMR (300 MHz, CDCl₃) δ: 1.49 (s, 9H), 2.43 (t, J₁=6.0 Hz, J₂=6.0 Hz, 4H), 3.71 (t, J₁=6.0 Hz, J₂=6.0 Hz, 4H).

### Step B: tert-Butyl 4-hydroxy-4-(nitromethyl)piperidine-1-carboxylate

To a mixture of tert-butyl 4-oxopiperidine-1-carboxylate (0.1 mol) and nitro-methane (0.1 mol) in methanol (200 mL) was added sodium methanolate (0.11 mol) at RT and the reaction was stirred for 1 h at room temperature. The solvent was evaporated. The residue was taken up into water, neutralized with acetic acid, extracted twice with EtOAc. The separated organic layer was washed with water, dried, filtered and evaporated to provide tert-butyl 4-hydroxy-4-(nitromethyl)piperidine-1-carboxylate. ¹H NMR (300 MHz, CDCl₃) δ: 1.46 (s, 9H), 1.61 (t, J₁=5.4 Hz, J₂=5.4 Hz, 4H), 2.92 (s, 1H), 3.19 (t, J₁=12.0 Hz, J₂=12.0 Hz, 2H), 3.94 (t, J₁=6.9 Hz, J₂=6.9 Hz, 2H), 4.43 (s, 2H).

### Step C: tert-Butyl 4-(aminomethyl)-4-hydroxypiperidine-1-carboxylate

The mixture of tert-butyl 4-hydroxy-4-(nitromethyl)piperidine-1-carboxylate (15.0 g, 0.058 mol) and acetic acid (12 mL) in methanol (180 mL) was hydrogenated at rt with palladium-on-carbon (10%, 1.5 g) as a catalyst. After uptake of hydrogen, the catalyst was filtered off and the filtrate was evaporated. The residue was taken up into ice water, alkalized with potassium hydroxide, extracted twice with EtOAc, dried over Na₂SO₄, filtered and concentrated to give tert-butyl 4-(aminomethyl)-4-hydroxypiperidine-1-carboxylate.

### Step D: tert-Butyl 4-((2-chloroacetamido)methyl)-4-hydroxypiperidine-1-carboxylate

The mixture of tert-butyl 4-(aminomethyl)-4-hydroxypiperidine-1-carboxylate (10.0 g, 45 mmol), chloroacetyl chloride (6 mL, 64 mmol) and K₂CO₃ (14.0 g, 95 mmol) in EtOAc/H₂O (100 mL/100 mL) was stirred for 1 h at 0° C. The crude mixture was extracted with EtOAc (2×300 mL). The combined organics were dried over Na₂SO₄, filtered and concentrated in vacuo to give tert-butyl 4-((2-chloroacetamido)methyl)-4-hydroxypiperidine-1-carboxylate. ¹H NMR (300 MHz, CDCl₃) δ: 1.45 (s, 9H), 1.53 (d, 4H), 2.59 (s, 1H), 3.21 (s, 2H), 3.35 (s, 2H), 3.78 (d, J=18.0 Hz, 2H), 4.13 (s, 2H), 6.99 (s, 1H).

### Step E: tert-Butyl 3-oxo-1-oxa-4,9-diazaspiro[5.5]undecane-9-carboxylate

To a mixture of potassium tert-butoxide (31.8 g, 283 mmol) and tert-butanol (500 mL) was added tert-butyl 4-((2-chloroacetamido)methyl)-4-hydroxypiperidine-1-carboxylate (41.9 g, 141 mmol) in THF (300 mL) over 40 minutes and the resulting mixture was continued to stir for 1 h at room temperature before it was concentrated. The residue was diluted with EtOAc and water, the organic layer was separated, washed with brine, and concentrated to provide tert-butyl 3-oxo-1-oxa-4,9-diazaspiro[5.5]undecane-9-carboxylate. ¹H NMR (300 MHz, CDCl₃) δ: 1.41 (s, 9H), 1.52 (s, 2H), 1.90 (d, J=12.0 Hz, 2H), 2.59 (s, 1H), 3.12 (m, 2H), 3.25 (s, 2H), 3.84 (d, J=6.4 Hz, 2H), 4.17 (s, 2H), 6.12 (s, 1H).

### Step F: tert-Butyl 1-oxa-4,9-diazaspiro[5.5]undecane-9-carboxylate

To a solution of tert-butyl 3-oxo-1-oxa-4,9-diazaspiro[5.5]undecane-9-carboxylate (16.0 g, 60 mmol) in THF (70 mL) was added tetrahydrofuran-borane (250 mL, 250 mmol) at room temperature. The reaction mixture was refluxed for 2 h and the solvent was removed under the reduced pressure. To the resulting mixture was added MeOH and N₁,N₁,N₂,N₂-tetramethylethane-1,2-diamine and the reaction was stirred at 78° C. overnight. The reaction was concentrated and the residue was diluted with EtOAc and water. The organic layer was separated, washed with brine, and concentrated in vacuo to give tert-butyl 1-oxa-4,9-diazaspiro[5.5]undecane-9-carboxylate. ¹H NMR (300 MHz, CDCl₃) δ: 1.45 (s, 9H), 1.61 (s, 2H), 1.93 (d, J=12.0 Hz, 2H), 2.67 (s, 2H), 2.83 (m, 2H), 3.16 (t, J₁=9.0 Hz, J₂=12.0 Hz, 2H), 3.65 (m, 4H).

### Intermediate 21

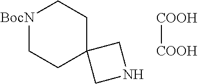

### Step A: methyl piperidine-4-carboxylate

To a solution of piperidine-4-carboxylic acid (400 g, 3.10 mol) in MeOH (3.5 L) was added SOCl₂ (550 g, 4.65 mol) dropwise at RT. The reaction mixture was then heated to 40-50° C. overnight. The next day, after TLC confirmed that the starting material was no longer present, the solvent was removed on a rotary evaporator to provide the title compound.

### Step B: piperidine-4-carboxamide

To a mixture of methyl piperidine-4-carboxylate (590 g, 3.10 mol) in NH₃/MeOH (4.5 L, V/V=40%) was added K₂CO₃ (5.0 g, 36 mmol). The resulting reaction mixture was heated to 140-150° C. overnight. The next day the solvent was removed on the rotary evaporator, and the crude product recrystallized by CH₃OH/EtOAc to afford the title compound.

### Step C: tert-butyl 4-cyanopiperidine-1-carboxylate

To POCl₃ (400 mL) was added piperidine-4-carboxamide (100 g, 0.78 mol) in portion. The reaction mixture was then heated to reflux for 3 h, cooled to RT, and filtered. The filtrate was evaporated on a vacuum distillation plant, and the residue was diluted with 250 mL CH₂Cl₂, and poured into 200 mL ice-water, stirred for 30 min and pH was adjusted to 9-10 by 40% NaOH (175 g). Then 300 mL acetone was added to make the reaction mixture homogeneous, and (Boc)₂O (202 g, 0.938 mol) was added (control pH=9-10). After addition the reaction mixture was stirred at rt for 1 h. H₂O (1 L) was added to make the salt dissolve, and the organic phase was separated. The aqueous phase was extracted by EtOAc (500 mL×2). The organic phases were combined, dried by Na₂SO₄, and evaporated on a rotary evaporator to get the crude product, which was passed through a silica gel column to provide the title compound. ¹H NMR (300 MHz, CDCl₃) δ 3.69-3.61 (m, 2H), 3.37-3.29 (m, 2H), 2.81-2.78 (m, 1H). 1.88-1.77 (m, 1H), 1.45 (s, 9H).

### Step D: tert-butyl 4-cyano-4-((tosyloxy)methyl)piperidine-1-carboxylate

Diisopropylamine (58 g) was added to anhydrous THF (600 mL) under N₂ protection, n-BuLi (2.5 M, 230 mL, 1.2 eq) was added at −40° C.˜−50° C. in 1, then the mixture was stirred at −30° C.˜−40° C. for 30 min. tert-Butyl 4-cyanopiperidine-1-carboxylate (100 g, 476 mmol, 1 eq.) in 400 mL anhydrous THF was added to the reaction mixture at about −60° C. to about −70° C. in 1 h, then stirred at about −50° C.˜to about 60° C. for 30 min. In another 2 L three-neck flask, (HCHO)n (58 g, 4 eq) was heated to get HCHO gas to pass through 1600 mL anhydrous THF to get a milky solution. The milky solution was added to the reaction mixture at about −40° C. to about −50° C. in 30 min and then warmed to −5° C. slowly. The reaction mixture was quenched with 50 mL H₂O. TsCl (109 g, 571 mmol, 1.2 eq.) was added to the reaction mixture, followed by Et₃N (96 g, 952 mmol, 2 eq.) within 30 min. The mixture was stirred overnight at rt. The next day the solvent was removed on the rotary evaporator, and the residue was passed through a silica gel column to yield the title compound. ¹H NMR (300 MHz, CDCl₃) δ 7.84-7.81 (m, 2H), 7.40-7.37 (m, 2H), 4.14 (m, 2H), 3.99 (m, 2H), 3.01 (m, 2H), 2.48 (s, 3H), 1.92-1.88 (m, 2H), 1.53-1.47 (m, 2H), 1.45 (s, 9H).

### Step E: tert-butyl 2,7-diazaspiro[3.5]nonane-7-carboxylate oxalate

To a solution of tert-butyl 4-cyano-4-((tosyloxy)methyl)piperidine-1-carboxylate (41 g, 104 mmol, 1 eq.) in anhydrous THF (600 mL) was added LiAlH₄ (4.7 g, 12 5 mmol, 1.2 eq.) in portion at 5-7° C., then it was stirred at 5-7° C. for 10 min. TLC confirmed that the raw material disappeared, then 4.7 g H₂O, 4.7 g 15% NaOH, and 14.1 g H₂O was added to quench the reaction. The mixture was filtered and the filtrate was washed by EtOAc (250 mL×6). The solvent was removed on the rotary evaporator to get the crude product, which was then dissolved in 45 mL CH₃OH, added the oxalic acid solution (13 g in 26 mL CH₃OH), and stirred for 30 min. Anhydrous ether (500 mL) was added, and white solid appeared, which was filtered, washed by ether (100 mL×3) to get the title compound. ¹H NMR (300 MHz, D₂O-d₆) δ 3.92 (s, 4H), 3.39-3.36 (t, 4H), 1.84-1.80 (q, 4H), 1.42 (s, 9H).

### Intermediate 22

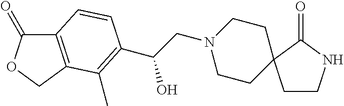 [SMILES: Cc1c([C@@H](O)CN2CCC3(CCNC3=O)CC2)ccc2c1COC2=O]

### Step A: 2,8-diazaspiro[4.5]decan-1-one hydrochloride

To a solution of tert-butyl 1-oxo-2,8-diazaspiro[4.5]decane-8-carboxylate (92 g, 0.36 mol) in CH₂Cl₂ (1 L) was slowly added a 4 M HCl solution (500 mL). The mixture was stirred for 8 h at RT. The mixture was concentrated under vacuum to afford the title compound. ¹H NMR (400 MHz, DMSO-d₆) δ 9.35 (s, 1H), 9.02 (s, 1H), 7.72 (s, 1H), 3.30-3.20 (m, 2H), 3.16 (m, J=6.8 Hz, 2H), 2.98-2.85 (m, 2H), 1.96 (m, J=6.8 Hz, 2H), 1.90-1.80 (m, 2H), 1.55 (d, J=14 Hz, 2H).

### Step B: (R)-8-(2-hydroxy-2-(4-methyl-1-oxo-1,3-dihydroisobenzofuran-5-yl)ethyl)-2,8-diazaspiro[4.5]decan-1-one

To a solution of 2,8-diazaspiro[4.5]decan-1-one hydrochloride (68 g, 0.35 mol) in ethanol (1.5 L) was added Et₃N (55 mL). The mixture was stirred for 2 hours. Then (R)-4-methyl-5-(oxiran-2-yl)isobenzofuran-1(3H)-one (65 g, 0.34 mol) was added. The mixture was heated to reflux for 40 h. After filtration, the solid was collected to provide the title compound. The filtrate was concentrated and purified by SFC separation to provide additional title compound. ¹H NMR (400 Hz, CDCl₃) δ 7.82-7.75 (m, 2H), 6.00 (s, 1H), 5.24 (s, 2H), 5.08 (dd, J=2.1 Hz and 10.4 Hz, 1H), 4.21 (s, 1H), 3.35 (t, J=6.8 Hz, 2H), 3.17-3.14 (m, 1H), 2.85-2.82 (m, 1H), 2.57 (dd, J=2.1 Hz and 10.4 Hz, 1H), 2.49 (t, J=8.8 Hz, 1H), 2.37 (t, J=10.8 Hz, 1H), 2.27 (s, 3H), 2.23 (J=6.8 Hz, 1H), 2.09-1.98 (m, 4H), 1.52 (t, J=12.8 Hz, 2H).

### Intermediate 23

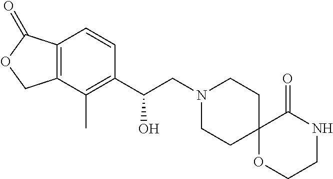 [SMILES: Cc1c([C@@H](O)CN2CCC3(CC2)OCCNC3=O)ccc2c1COC2=O]

### Step A: 1-oxa-4,9-diazaspiro[5.5]undecan-5-one

The mixture of tert-butyl 5-oxo-1-oxa-4,9-diazaspiro[5.5]undecane-9-carboxylate (1.00 g, 3.70 mmol) in DCM/TFA (1:1, 30 mL) was stirred at room temperature for 1 hour. The mixture was concentrated to afford the title compound as a TFA salt.

### Step B: (R)-9-(2-hydroxy-2-(4-methyl-1-oxo-1,3-dihydroisobenzofuran-5-yl)ethyl)-1-oxa-4,9-diazaspiro[5.5]undecan-5-one

The mixture of 1-oxa-4,9-diazaspiro[5.5]undecan-5-one (1.00 g, 3.52 mmol) and TEA (1.07 g, 10.6 mmol), (R)-4-methyl-5-(oxiran-2-yl)isobenzofuran-1(3H)-one (736 mg, 3.87 mmol) in EtOH (35 mL) was stirred at 90° C. for 20 h. The mixture was concentrated and the residue was purified by chromatography on silica gel (DCM/MeOH=10/1) to afford the title compound. LC-MS (ESI, m/z): 361.2 [M+1]⁺.

The following intermediates in Table 2 were prepared according to the method described for INTERMEDIATE 23 using (R)-4-methyl-5-(oxiran-2-yl)isobenzofuran-1(3H)-one (INTERMEDIATE 4A) and the spirocycles specified below.

TABLE 2

| Int. # | Starting Material | Intermediate Structure | LC-MS[M + 1]⁺ |
|---|---|---|---|
| 24 | 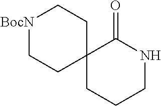 [SMILES: CC(C)(C)OC(=O)N1CCC2(CCCNC2=O)CC1] | 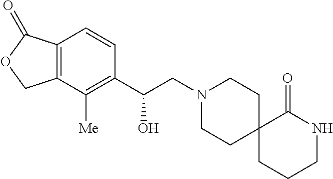 [SMILES: Cc1c([C@@H](O)CN2CCC3(CCCNC3=O)CC2)ccc2c1COC2=O] | 359 |
| | tert-butyl 1-oxo-2,9- | (R)-9-(2-hydroxy-2-(4-methyl-oxo- | |
| | diazaspiro[5.5]undecane-9- | 1,3-dihydroisobenzofuran-5-yl)ethyl)- | |
| | carboxylate (Shanghai AQ | 2,9-diazaspiro[5.5]undecan-1-one | |
| | BioPharma Co., Ltd, catalog # | | |
| | ABP3640) | | |
| 25 | 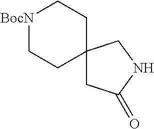 [SMILES: CC(C)(C)OC(=O)N1CCC2(CC1)CNC(=O)C2] | 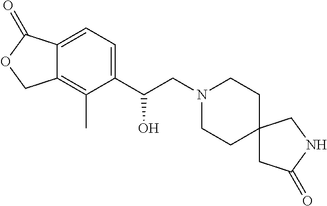 | 345 |
| | 3-oxo-2,8-diaza- | (R)-8-(2-hydroxy-2-(4-methyl-oxo- | |
| | spiro[4,5]decane-8-carboxylic | 1,3-dihydroisobenzofuran-5-yl)ethyl)- | |
| | acid tert-butyl ester | 2,8-diazaspiro[4.5]decan-3-one | |

### Intermediate 26

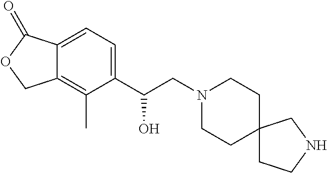

### Step A: (R)-tert-butyl 8-(2-hydroxy-2-(4-methyl-1-oxo-1,3-dihydroisobenzofuran-5-yl)ethyl)-2,8-diazaspiro[4.5]decane-2-carboxylate

To (R)-4-methyl-5-(oxiran-2-yl)isobenzofuran-1(3H)-one (3.8 g, 20 mmol) in EtOH (42 mL) was added tert-butyl 2,8-diazaspiro[4.5]decane-2-carboxylate (4.0 g, 16.6 mmol). The reaction mixture was heated at 90° C. overnight. The reaction mixture was concentrated, and purified by silica gel column chromatography (0-10% MeOH/EtOAc) to give the title compound. LC/MS: [(M+1)]⁺=431.

### Step B: (R)-5-(1-hydroxy-2-(2,8-diazaspiro[4.5]decan-8-yl)ethyl)-4-methylisobenzofuran-1(3H)-one

To (R)-tert-butyl 8-(2-hydroxy-2-(4-methyl-1-oxo-1,3-dihydroisobenzofuran-5-yl)ethyl)-2,8-diazaspiro[4.5]decane-2-carboxylate (500 mg, 1.16 mmol) in DCM (5.8 mL) was added TFA (1.8 mL, 23.2 mmol) at 0° C. The reaction mixture was stirred at RT for 1 h. The reaction mixture was concentrated, and azeotroped with DCM/MeOH/PhMe to remove as much TFA as possible. Then a 10 g Bond Elut SCX was first rinsed with MeOH, load sample with MeOH, washed with MeOH dropwise to remove TFA, finally rinsed with 2N NH₃/MeOH to get the title compound as free amine. LC/MS: [(M+1)]⁺=331.

### Intermediate 27

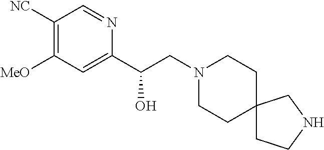 [SMILES: COc1cc([C@@H](O)CN2CCC3(CCNC3)CC2)ncc1C#N]

### Step A: tert-Butyl 8-(2-(5-cyano-4-methoxypyridin-2-yl)-2-hydroxyethyl)-2,8-diazaspiro[4.5]decane-2-carboxylate

A microwave tube was charged with tert-butyl 2,8-diazaspiro[4.5]decane-2-carboxylate (0.500 g, 2.08 mmol), 4-methoxy-6-(oxiran-2-yl)pyridine-3-carbonitrile (Intermediate 14 slow eluting isomer B, 0.367 g, 2.08 mmol), and ethanol (4.0 mL). The solution was degassed and filled with nitrogen (3×), then sealed and heated in a microwave reactor to 140° C. for 1 hr. The reaction was cooled to RT and concentrated in vacuo. The resulting residue was purified by prep TLC (2% MeOH:DCM) to provide tert-butyl 8-(2-(5-cyano-4-methoxypyridin-2-yl)-2-hydroxyethyl)-2,8-diazaspiro[4.5]decane-2-carboxylate.

¹H NMR (500 MHz, CDCl₃), δ 8.44 (s, 1H), 7.21 (s, 1H), 4.71 (m, 1H), 3.95 (s, 3H), 3.28 (m, 2H), 3.09 (m, 2H), 2.78 (m, 1H), 2.65 (m, 1H), 2.48 (m, 1H), 2.37 (m, 3H), 1.58 (m, 4H), 1.38 (s, 9H); LC-MS (IE, m/z): 417 [M+1]⁺.

### Step B: (S)-6-(1-hydroxy-2-(2,8-diazaspiro[4.5]decan-8-yl)ethyl)-4-methoxynicotinonitrile

To a solution of tert-butyl 8-(2-(5-cyano-4-methoxypyridin-2-yl)-2-hydroxyethyl)-2,8-diazaspiro[4.5]decane-2-carboxylate (0.500 mg, 1.20 mmol) in dichloromethane (4.0 mL) at 0° C. was added trifluoromethylacetic acid (2.0 mL). The reaction was stirred at room temperature for 30 min. The mixture was concentrated in vacuo and dried under high vacuum. The resulting residue was partitioned between DCM and saturated sodium bicarbonate solution which was adjusted with 1N NaOH to maintain pH about 9. The aqueous layer was extracted with iPrOH:CHCl₃ (1:3, 3×) and the combined organic layers were washed with brine, dried (Mg₂SO₄), filtered and concentrated to provide 6-(1-hydroxy-2-(2,8-diazaspiro[4.5]decan-8-yl)ethyl)-4-methoxynicotinonitrile. LC-MS (IE, m/z): 317 [M+1]⁺.

### Intermediate 28

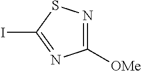

To a stirred solution of iodine (484 mg, 1.91 mmol) and isoamyl nitrite (410 μL, 3.05 mmol) in acetonitrile (6 mL) was added dropwise an acetonitrile (2 mL) solution of 3-methoxy-1,2,4-thiadiazol-5-amine (100 mg, 0.762 mmol). The reaction mixture was monitored by TLC and LCMS. After the starting material was consumed, the reaction mixture was absorbed on silica. Purification by silica gel column chromatography (0-30% EtOAc/hex) gave 5-iodo-3-methoxy-1,2,4-thiadiazole. LC/MS: [(M+1)]⁺=243.

### Intermediate 29

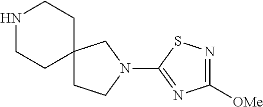 [SMILES: COc1nsc(N2CCC3(CCNCC3)C2)n1]

### Step A: tert-butyl 2-(3-methoxy-1,2,4-thiadiazol-5-yl)-2,8-diazaspiro[4.5]decane-8-carboxylate

A microwave vial was charged with a magnetic stir bar, tert-butyl 2,8-diazaspiro[4.5]decane-8-carboxylate (commercially available from multiple vendors, 120 mg, 0.499 mmol), 5-iodo-3-methoxy-1,2,4-thiadiazole (133 mg, 0.549 mmol), tribasic potassium phosphate (212 mg, 0.999 mmol), Pd₂(dba)₃ (22.9 mg, 0.025 mmol), and XPhos (47.6 mg, 0.100 mmol). The vial was sealed, degassed, and filled with dioxane (3.3 mL). The reaction mixture was heated at 95° C. overnight, filtered and rinsed with DCM. The filtrate was concentrated and the crude product was purified by silica gel column chromatography (0-10% MeOH/DCM) to give the title compound. LC-MS 355 [M+1]⁺.

### Step B: 3-methoxy-5-(2,8-diazaspiro[4.5]decan-2-yl)-1,2,4-thiadiazole

tert-Butyl 2-(3-methoxy-1,2,4-thiadiazol-5-yl)-2,8-diazaspiro[4.5]decane-8-carboxylate (150.9 mg, 0.426 mmol) was dissolved in DCM (2 mL), and TFA (0.984 mL, 12.8 mmol) was added to free Boc protection and yield the TFA salt. After evaporation of the solvents, the residue was dissolved in CHCl₃:IPA (3:1). This organic phase was washed with two portions of 0.1 N NaOH, then with one portion of brine. The organic phase was collected and dried over MgSO₄. Excess solvent was removed under reduced pressure to afford the title compound as a free base. LC-MS 255 [M+1]⁺.

The following amines provide in Table 3 were prepared in an analogous fashion to the method described for Intermediate 29, starting from intermediates specified below.

TABLE 3

| | | | | LC-MS |
|---|---|---|---|---|
| Int. | Starting material | Starting Material | Intermediate Structure | [M + 1]+ |
| 30 | 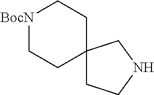 [SMILES: CC(C)(C)OC(=O)N1CCC2(CCNC2)CC1] | 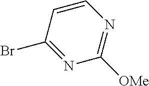 | 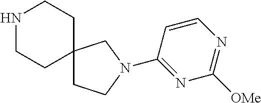 [SMILES: COc1nccc(N2CCC3(CCNCC3)C2)n1] | 249 |
| | tert-butyl 2,8- | 4-bromo-2- | 2-(2-methoxypyrimidin-4-yl)-2,8- | |
| | diazaspiro[4.5]decane- | methoxypyrimidine | diazaspiro[4.5]decane | |
| | 8-carboxylate | | | |
| 31 | 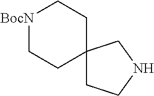 [SMILES: CC(C)(C)OC(=O)N1CCC2(CCNC2)CC1] | 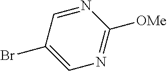 | 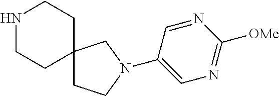 [SMILES: COc1ncc(N2CCC3(CCNCC3)C2)cn1] | 249 |
| | tert-butyl 2,8- | 5-bromo-2- | 2-(2-methoxypyrimidin-5-yl)-2,8- | |
| | diazaspiro[4.5]decane- | methoxypyrimidine | diazaspiro[4.5]decane | |
| | 8-carboxylate | | | |
| 32 | 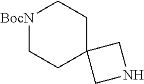 [SMILES: CC(C)(C)OC(=O)N1CCC2(CC1)CNC2] | 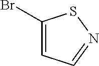 | 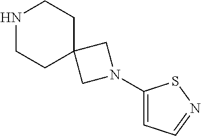 [SMILES: c1cc(N2CC3(CCNCC3)C2)sn1] | 310 (for Boc protected intermediate) |
| | tert-butyl 2,7- | 5-bromoisothiazole | 5-(2,7-diazaspiro[3.5]nonan-2- | |
| | diazaspiro[3.5]nonane- | | yl)isothiazole | |
| | 7-carboxylate | | | |

### Intermediate 33

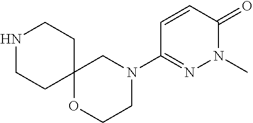 [SMILES: Cn1nc(N2CCOC3(CCNCC3)C2)ccc1=O]

### Step A: tert-butyl 4-(1-methyl-6-oxo-1,6-dihydropyridazin-3-yl)-1-oxa-4,9-diazaspiro[5.5]undecane-9-carboxylate

In the reaction vessel RuPhos Indoline Precatalyst (0.058 g, 0.079 mmol) and sodium tert-butoxide (0.305 g, 3.17 mmol) were combined, followed by 6-bromo-2-methylpyridazin-3(2H)-one (0.300 g, 1.587 mmol) and tert-butyl 1-oxa-4,9-diazaspiro[5.5]undecane-9-carboxylate (0.488 g, 1.905 mmol). This mixture was then evacuated and backfilled with N₂ (3 times). Then dry, degassed tetrahydrofuran (7.9 mL) was added to this flask. This mixture was heated at 80° C. overnight. The mixture was cooled and solvent was removed on a rotavapor. The residue was dissolved in 40 mL of EtOAc and 20 mL of water. After separation, aqueous layer was extracted with EtOAc (20 mL×2). The organic layers were combined and washed with 20 mL brine. Then it was dried over Na₂SO₄. Removing solvent gave the crude product, which was purifed by column chromatography (40 g slica gel column, eluted with 100% EtOAc) to afford the title compound. LC-MS (IE, m/z): 365 (M+1)⁺.

### Step B: 2-methyl-6-(1-oxa-4,9-diazaspiro[5.5]undecan-4-yl)pyridazin-3(2H)-one

The title compound was prepared in a similar fashion to that described for INTERMEDIATE 27 above using TFA. LC-MS (IE, m/z): 265 (M+1)⁺.

### Intermediate 34

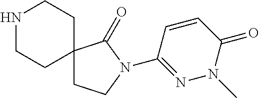 [SMILES: Cn1nc(N2CCC3(CCNCC3)C2=O)ccc1=O]

### Step A: tert-butyl 2-(1-methyl-6-oxo-1,6-dihydropyridazin-3-yl)-1-oxo-2,8-diazaspiro[4.5]decane-8-carboxylate

To a reaction flask was charged tert-butyl 1-oxo-2,8-diazaspiro[4.5]decane-8-carboxylate (2.00 g, 7.86 mmol), 6-bromo-2-methylpyridazin-3(2H)-one (1.64 mg, 8.65 mmol), Pd₂(dba)₃ (180 mg, 0.197 mmol), Xantphos (341 mg, 0.590 mmol), and Cs₂CO₃ (5.12 g, 15.7 mmol). The flask was sealed, degased, and filled with anhydrous dioxane (26 mL). The reaction mixture was heated at 90° C. overnight, diluted with EtOAc and DCM, then filtered through CELITE®. After solvent evaporation, the crude product was purified by silica gel column chromatography (0-10% MeOH/DCM as eluent) to afford the title compound. LC/MS: (M+1)⁺: 363.0. ¹H NMR (500 MHz, CDCl₃), δ 8.50 (d, J=10.0 Hz, 1H), 6.97 (d, J=10.0 Hz, 1H), 4.02 (br s, 2H), 3.86 (t, J=7.0 Hz, 2H), 3.74 (s, 3H), 3.05-3.00 (m, 2H), 2.06-2.02 (m, 2H), 1.98-1.94 (m, 2H), 1.58-1.50 (m, 2H), 1.49 (s, 9H).

### Step B: 2-(1-methyl-6-oxo-1,6-dihydropyridazin-3-yl)-2,8-diazaspiro[4.5]decan-1-one

tert-Butyl 2-(1-methyl-6-oxo-1,6-dihydropyridazin-3-yl)-1-oxo-2,8-diazaspiro[4.5]decane-8-carboxylate (2.55 g, 7.04 mmol) in DCM (12 mL) was treated with TFA (8.1 mL, 106 mmol) at 0° C. to free Boc protection and give TFA salt. Then two 10 g Bond Elut SCX columns were first rinsed with MeOH, load sample with MeOH, washed with MeOH dropwise to remove TFA, finally rinsed with 2N NH₃/MeOH to get the title compound as a free amine. LC/MS: (M+1)⁺: 263.05

### Intermediate 35

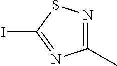

5-iodo-3-methyl-1,2,4-thiadiazole was prepared by the same condition as INTERMEDIATE 28 starting from 3-methyl-1,2,4-thiadiazol-5-amine. ¹H NMR (500 MHz, CDCl₃) δ 2.72 (s, 3H).

### Intermediate 36

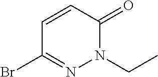

6-Bromo-3-pyridazinol (200 mg, 1.14 mmol), potassium carbonate (316 mg, 2.29 mmol), and anhydrous acetonitrile (5.7 mL) were charged to a round-bottom flask equipped with a rubber septum and magnetic stir bar. Ethyl iodide (102 μL, 1.26 mmol) was charged dropwise via syringe. The flask was then equipped with a reflux condenser, then heated at reflux for 3 h. The flask was cooled to room temperature and then monitored by TLC and LCMS. The reaction mixture was filtered through CELITE® and the flask was rinsed with methanol. The filtrate containing the crude product was concentrated and purified on silica gel column chromatography (0-10% MeOH:DCM) to afford 6-bromo-2-ethylpyridazin-3(2H)-one. LC/MS: (M+2)⁺: 204.81.

### Intermediate 37

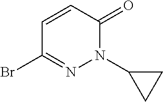 [SMILES: O=c1ccc(Br)nn1C1CC1]

6-Bromopyridazin-3(2H)-one (100 mg, 0.571 mmol), cyclopropylboronic acid (73.6 mg, 0.857 mmol), diacetoxycopper (208 mg, 1.14 mmol), pyridine (368 μL, 4.57 mmol), triethylamine (399 μL, 2.86 mmol) and anhydrous THF (2.8 mL) were charged to a microwave vial equipped with a rubber septum and magnetic stirbar. Under air, the reaction mixture was microwaved at 140° C. for 10 min. The flask was cooled to room temperature and then monitored by TLC and LCMS. The reaction mixture was filtered through CELITE® and the flask was rinsed with methanol. The filtrate containing the crude product was concentrated and purified on silica gel column chromatography (0-10% MeOH:DCM) to afford 6-bromo-2-cyclopropylpyridazin-3(2H)-one. LC/MS: (M+2)⁺: 216.85.

### Intermediate 38

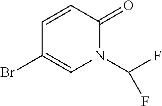 [SMILES: O=c1ccc(Br)cn1C(F)F]

### Step A: 5-bromopyridin-2-ol

Into a 10 L round-bottom flask, was placed a solution of H₂SO₄ (480 ml) in H₂O (6000 ml). 5-Bromopyridin-2-amine (400 g, 2.31 mol, 1.00 equiv) was added to the mixture. The reaction mixture was allowed to react with stirring for 10 minutes and it was cooled to −10° C. A solution of NaNO₂ (180 g, 2.61 mol, 1.09 equiv) in H₂O (1200 ml) was added drop-wise to the mixture with stirring, while cooling the reaction mixture to a temperature of 0-5° C. The resulting solution was allowed to react, with stirring, for 0.5 hours while the temperature was maintained at 0-5° C. and an additional 0.5 hours while the temperature was maintained at room temperature. The reaction mixture was filtered and the filter cake was washed three times with water; this resulted in the title compound

### Step B: 5-bromo-1-(difluoromethyl)pyridin-2(1H)-one

A solution of 5-bromopyridin-2-ol (200 g, 1.15 mol, 1.00 equiv) in DMSO (2 L) was placed into a 3 L 3-necked round-bottom flask purged and maintained with an inert atmosphere of nitrogen. To this mixture was added 60% NaH (50 g) and the mixture was allowed to react for 15 minutes. Sodium 2-chloro-2,2-difluoroacetate (200 g, 1.32 mol, 1.14 equiv) was added to the mixture and the resulting solution was allowed to react, with stirring, for 3.5 hours while the temperature was maintained at 55-65° C. in a bath of oil. The reaction mixture was cooled in a bath of H₂O. The reaction mixture was then quenched by the adding 5 L of H₂O. The resulting solution was extracted three times with 3 L of EtOAc and the organic layers combined and dried over Na₂SO₄. A filtration was performed and the filtrate was concentrated by evaporation under vacuum using a rotary evaporator. The residue was purified by eluting through a column with a 1:10 EtOAc/PE solvent system. This resulted in the title compound. ¹H NMR (300 MHz, CDCl₃): δ 6.50 (1H, d), 7.41 (1H, d), 7.56 (1H, s), 7.60 (1H, m); ¹³C NMR (75 MHz, CDCl₃): δ 99.53, 107.2, 122.9, 129.3, 144.2, 159.2.

### Intermediate 39

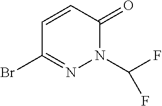 [SMILES: O=c1ccc(Br)nn1C(F)F]

To a flask was charged 3-bromo-6-chloropyridazine (300 mg, 1.55 mmol), 2,2-difluoro-2-(fluorosulfonyl)acetic acid (829 mg, 4.65 mmol), and sodium hydrogencarbonate (130 mg, 1.55 mmol). The flask was filled with acetonitrile (7.8 mL). The reaction mixture was heated at 50° C. for 3 h, and filtered through CELITE®. The filtrate was evaporated to give the crude product, which was purified by silica gel column chromatography (0-100% EtOAc/Hex) to afford 6-bromo-2-(difluoromethyl)pyridazin-3(2H)-one. LC/MS: (M+1)⁺: 224.86.

### Intermediate 40

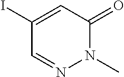

5-Iodopyridazin-3(2H)-one (200 mg, 0.90 mmol), potassium carbonate (249 mg, 1.80 mmol), and anhydrous acetonitrile (4.5 mL) were charged to a round-bottom flask equipped with a rubber septum and magnetic stirbar. Methyl iodide (62.0 μL, 0.991 mmol) was charged dropwise via syringe. The flask was then equipped with a reflux condenser and heated at reflux for 1 hour. The reaction mixture was filtered through CELITE®. The filtrate was evaporated to give the crude product, which was purified by silica gel column chromatography (0-10% MeOH/DCM) to afford 5-iodo-2-methylpyridazin-3(2H)-one. LC/MS: (M+1)⁺: 236.80.

### Intermediate 41

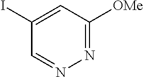

5-Iodopyridazin-3(2H)-one (200 mg, 0.901 mmol) and methanol (54.7 μL, 1.35 mmol) were charged to a microwave vial equipped with a magnetic stirbar. This was dissolved in THF (4.5 mL). To this solution was charged di-tert-butyl azodicarboxylate (311 mg, 1.35 mmol) and triphenylphosphine (354 mg, 1.35 mmol). The reaction mixture was then equipped with a rubber septum and then stirred at room temperature overnight. The reaction mixture was quenched with MeOH and then then concentrated under reduced pressure to give the crude product, which was purified by silica gel column chromatography (0-10% MeOH/DCM) to afford 5-iodo-3-methoxypyridazine. LC/MS: (M+1)⁺: 236.83.

### Intermediate 42

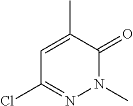

6-Chloro-2,4-dimethylpyridazin-3(2H)-one was prepared with the same condition as INTERMEDIATE 40 using 6-chloro-4-methylpyridazin-3(2H)-one. LC/MS: (M+1)⁺: 158.90.

### Intermediate 43

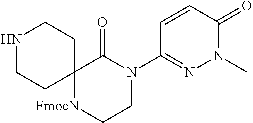

### Step A: 1-((9H-fluoren-9-yl)methyl) 9-tert-butyl 5-oxo-1,4,9-triazaspiro[5.5]undecane-1,9-dicarboxylate

At 0° C. to a dioxane (10 mL) and water (5 mL) solution of commercially available (ChemBridge Building Block Library catalog #4042448; Aldrich catalog # CDS019358) tert-butyl 5-oxo-1,4,9-triazaspiro[5.5]undecane-9-carboxylate (1.00 g, 3.71 mmol) was added sodium bicarbonate (0.624 g, 7.43 mmol), followed by 9-fluorenylmethyl chloroformate (0.960 g, 3.71 mmol, drop wise over 30 min period with syringe pump). The reaction mixture was allowed to stir at ambient temperature for 2 hours and then concentrated in vacuo (dioxane removal). The remaining aqueous layer was then extracted with DCM (3×15 mL). The combined organic layers were then dried over sodium sulfate, filtered and concentrated in vacuo. The crude residue was purified via MPLC (0-100% EtOAc/Hex gradient) to afford the title compound. LC/MS: (M+1)⁺: 492.11.

### Step B: 1-((9H-fluoren-9-yl)methyl) 9-tert-butyl 4-(1-methyl-6-oxo-1,6-dihydropyridazin-3-yl)-5-oxo-1,4,9-triazaspiro[5.5]undecane-1,9-dicarboxylate

The title compounds was prepared in an analogous fashion to INTERMEDIATE 34 (Step A) starting from 1-((9H-fluoren-9-yl)methyl) 9-tert-butyl 5-oxo-1,4,9-triazaspiro[5.5]undecane-1,9-dicarboxylate and and 6-bromo-2-methylpyridazin-3(2H)-one. LC/MS: (M+1)⁺: 600.28.

### Step C: (9H-fluoren-9-yl)methyl 4-(1-methyl-6-oxo-1,6-dihydropyridazin-3-yl)-5-oxo-1,4,9-triazaspiro[5.5]undecane-1-carboxylate

To a solution of 1-((9H-fluoren-9-yl)methyl) 9-tert-butyl 4-(1-methyl-6-oxo-1,6-dihydropyridazin-3-yl)-5-oxo-1,4,9-triazaspiro[5.5]undecane-1,9-dicarboxylate (33 mg, 0.055 mmol) in MeOH (10 mL) was added HCl (0.069 mL, 4 M in dioxane, 0.275 mmol). After 4 h, the reaction mixture was evaporated to afford the HCl salt of the title compound. LC/MS: (M+1)⁺: 500.07.

### Intermediate 44

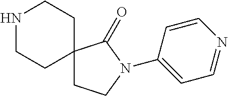 [SMILES: O=C1N(c2ccncc2)CCC12CCNCC2]

### Step A: tert-butyl 1-oxo-2-(pyridin-4-yl)-2,8-diazaspiro[4.5]decane-8-carboxylate

To a microwave vial was charged tert-butyl 1-oxo-2,8-diazaspiro[4.5]decane-8-carboxylate (80 mg, 0.315 mmol), 4-iodopyridine (64.5 mg, 0.315 mmol), copper(I) iodide (3.00 mg, 0.016 mmol), (1R,2R)—N1,N2-dimethylcyclohexane-1,2-diamine (9.92 μL, 0.063 mmol), and potassium phosphate (134 mg, 0.629 mmol). The vial was sealed, degased, and filled with toluene (1.6 mL). The reaction mixture was heated at 110° C. for 24 h, diluted with water, and extracted with EtOAc. The organic layer was washed with brined, dried, evaporated to give the crude product, which was purified by silica gel column chromatography (0-10% MeOH/DCM) to give the title compound. LC/MS: (M+1)⁺: 332.05.

### Step B: 2-(pyridin-4-yl)-2,8-diazaspiro[4.5]decan-1-one

The title compounds was prepared in an analogous fashion to INTERMEDIATE 34 (Step B) starting from tert-butyl 1-oxo-2-(pyridin-4-yl)-2,8-diazaspiro[4.5]decane-8-carboxylate. LC/MS: (M+1)⁺: 232.05.

The amines intermediates were prepared in an analogous fashion to the method described for Intermediate 34 or 44, using the starting materials specified below.

TABLE 4

| Int. | Starting Materials | Intermediate Structure | LC-MS [M + 1]⁺ |
|---|---|---|---|
| 45 | 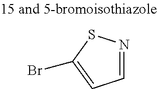 | 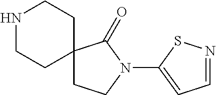 [SMILES: O=C1N(c2ccns2)CCC12CCNCC2] | 238.41 |
| | Method for Int. 34 | 2-(isothiazol-5-yl)-2,8- | |
| | | diazaspiro[4.5]decan-1-one | |
| 46 | 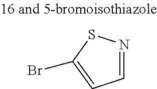 | 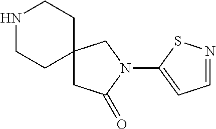 [SMILES: O=C1CC2(CCNCC2)CN1c1ccns1] | 238.43 |
| | Method for Int. 34 | 2-(isothiazol-5-yl)-2,8- | |
| | | diazaspiro[4.5]decan-3-one | |
| 47 | 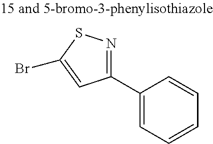 [SMILES: Brc1cc(-c2ccccc2)ns1] | 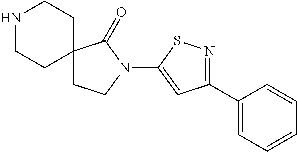 [SMILES: O=C1N(c2cc(-c3ccccc3)ns2)CCC12CCNCC2] | 314 |
| | Method for Int. 34 | 2-(3-phenylisothiazol-5-yl)-2,8- | |
| | | diazaspiro[4.5]decan-1-one | |
| 48 | 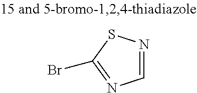 | 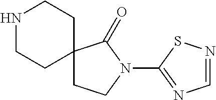 [SMILES: O=C1N(c2ncns2)CCC12CCNCC2] | 239.01 |
| | Method for Int. 34 | 2-(1,2,4-thiadiazol-5-yl)-2,8- | |
| | | diazaspiro[4.5]decan-1-one | |
| 49 | 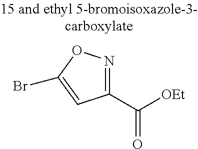 [SMILES: CCOC(=O)c1cc(Br)on1] | 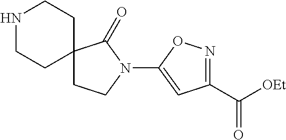 [SMILES: CCOC(=O)c1cc(N2CCC3(CCNCC3)C2=O)on1] | 294 |
| | Method for Int. 34 | ethyl 5-(1-oxo-2,8- | |
| | | diazaspiro[4.5]decan-2-yl)isoxazole- | |
| | | 3-carboxylate | |
| 50 | 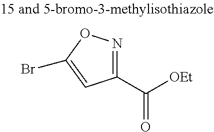 [SMILES: CCOC(=O)c1cc(Br)on1] | 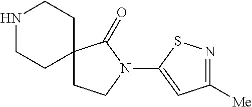 [SMILES: Cc1cc(N2CCC3(CCNCC3)C2=O)sn1] | 251.97 |
| | Method for Int. 34 | 2-(3-methylisothiazol-5-yl)-2,8- | |
| | | diazaspiro[4.5]decan-1-one | |
| 51 | 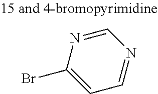 | 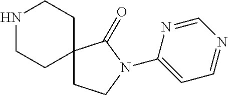 [SMILES: O=C1N(c2ccncn2)CCC12CCNCC2] | 233.03 |
| | Method for Int. 34 | 2-(pyrimidin-4-yl)-2,8- | |
| | | diazaspiro[4.5]decan-1-one | |
| 52 | 15 and 35; Method for Int. 34 | 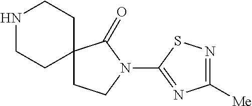 [SMILES: Cc1nsc(N2CCC3(CCNCC3)C2=O)n1] | 253.27 |
| | | 2-(3-methyl-1,2,4-thiadazol-5-yl)- | |
| | | 2,8-diazaspiro[4.5]decan-1-one | |
| 53 | 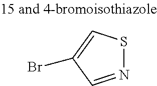 | 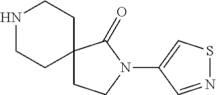 [SMILES: O=C1N(c2cnsc2)CCC12CCNCC2] | 237.96 |
| | Method for Int. 34 | 2-(isothiazol-4-yl)-2,8- | |
| | | diazaspiro[4.5]decan-1-one | |
| 54 | 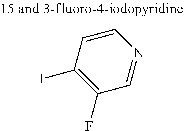 | 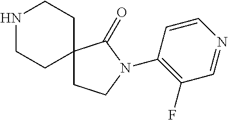 [SMILES: O=C1N(c2ccncc2F)CCC12CCNCC2] | 250.02 |
| | Method for Int. 44 | 2-(3-fluoropyridin-4-yl)-2,8- | |
| | | diazaspiro[4.5]decan-1-one | |
| 55 | 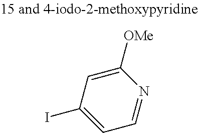 | 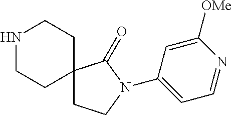 [SMILES: COc1cc(N2CCC3(CCNCC3)C2=O)ccn1] | 262.05 |
| | Method for Int. 44 | 2-(2-methoxypyridin-4-yl)-2,8- | |
| | | diazaspiro[4.5]decan-1-one | |
| 56 | 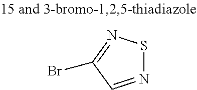 | 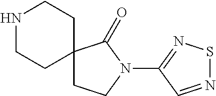 [SMILES: O=C1N(c2cnsn2)CCC12CCNCC2] | 239.00 |
| | Method for Int. 44 | 2-(1,2,5-thiadazol-3-yl)-2,8- | |
| | | diazaspiro[4.5]decan-1-one | |
| 57 | 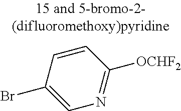 [SMILES: FC(F)Oc1ccc(Br)cn1] | 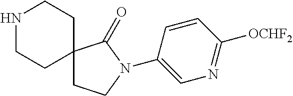 [SMILES: O=C1N(c2ccc(OC(F)F)nc2)CCC12CCNCC2] | 298.03 |
| | Method for Int. 44 | 2-(6-(difluoromethoxy)pyridin-3-yl)- | |
| | | 2,8-diazaspiro[4.5]decan-1-one | |
| 58 | 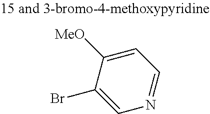 | 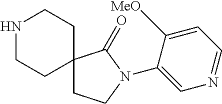 [SMILES: COc1ccncc1N1CCC2(CCNCC2)C1=O] | 262.00 |
| | Method for Int. 44 | 2-(4-methoxypyridin-3-yl)-2,8- | |
| | | diazaspiro[4.5]decan-1-one | |
| 59 | 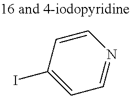 | 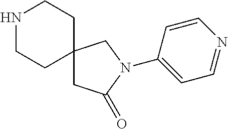 [SMILES: O=C1CC2(CCNCC2)CN1c1ccncc1] | 232.05 |
| | Method for Int. 34 | 2-(pyridin-4-yl)-2,8- | |
| | | diazaspiro[4.5]decan-3-one | |
| 60 | 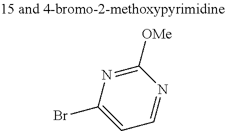 | 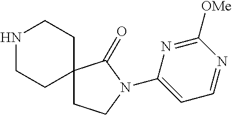 [SMILES: COc1nccc(N2CCC3(CCNCC3)C2=O)n1] | 263.06 |
| | Method for Int. 34 | 2-(2-methoxypyrimidin-4-yl)-2,8- | |
| | | diazaspiro[4.5]decan-1-one | |
| 61 | 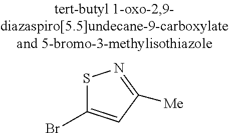 | 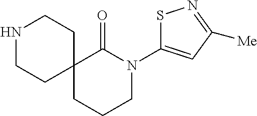 [SMILES: Cc1cc(N2CCCC3(CCNCC3)C2=O)sn1] | 266.03 |
| | Method for Int. 34 | 2-(3-methylisothiazol-5-yl)-2,9- | |
| | | diazaspiro[5.5]undecan-1-one | |
| 62 | 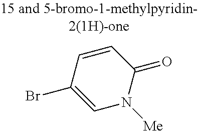 | 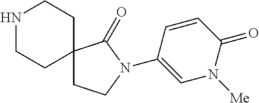 [SMILES: Cn1cc(N2CCC3(CCNCC3)C2=O)ccc1=O] | 262.06 |
| | Method for Int. 44 | 2-(1-methyl-6-oxo-1,6- | |
| | | dihydropyridin-3-yl)-2,8- | |
| | | diazaspiro[4.5]decan-1-one | |
| 63 | 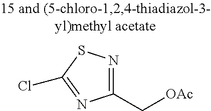 [SMILES: CC(=O)OCc1nsc(Cl)n1] | 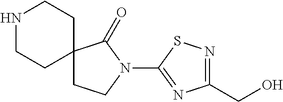 [SMILES: O=C1N(c2nc(CO)ns2)CCC12CCNCC2] | 268.99 |
| | Method for Int. 44 | 2-(3-(hydroxymethyl)-1,2,4- | |
| | | thiadiazol-5-yl)-2,8- | |
| | | diazaspiro[4.5]decan-1-one | |
| 64 | 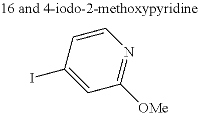 | 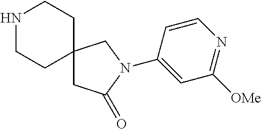 [SMILES: COc1cc(N2CC3(CCNCC3)CC2=O)ccn1] | 262.01 |
| | Method for Int. 34 | 2-(2-methoxypyridin-4-yl)-2,8- | |
| | | diazaspiro[4.5]decan-3-one | |
| 65 | 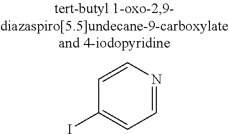 | 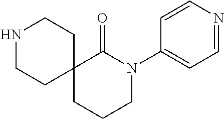 [SMILES: O=C1N(c2ccncc2)CCCC12CCNCC2] | 246.12 |
| | Method for Int. 34 | 2-(pyridin-4-yl)-2,9- | |
| | | diazaspiro[5.5]undecan-1-one | |
| 66 | 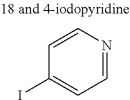 | 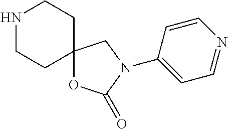 [SMILES: O=C1OC2(CCNCC2)CN1c1ccncc1] | 234.07 |
| | Method for Int. 34 | 3-(pyridin-4-yl)-1-oxa-3,8- | |
| | | diazaspiro[4.5]decan-2-one | |
| 67 | 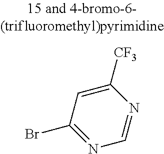 [SMILES: FC(F)(F)c1cc(Br)ncn1] | 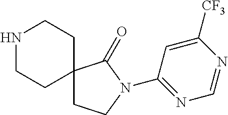 [SMILES: O=C1N(c2cc(C(F)(F)F)ncn2)CCC12CCNCC2] | 301.4 |
| | Method for Int. 34 | 2-(6-(trifluoromethyl)pyrimidin-4- | |
| | | yl)-2,8-diazaspiro[4.5]decan-1-one | |
| 68 | 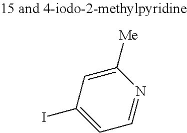 | 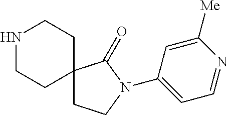 [SMILES: Cc1cc(N2CCC3(CCNCC3)C2=O)ccn1] | 246.08 |
| | Method for Int. 34 | 2-(2-methylpyridin-4-yl)-2,8- | |
| | | diazaspiro[4.5]decan-1-one | |
| 69 | 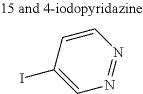 | 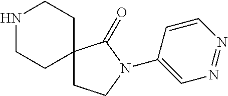 [SMILES: O=C1N(c2ccnnc2)CCC12CCNCC2] | 232.96 |
| | Method for Int. 34 | 2-(pyridazin-4-yl)-2,8- | |
| | | diazaspiro[4.5]decan-1-one | |
| 70 | 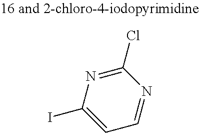 | 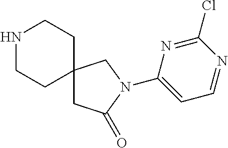 [SMILES: O=C1CC2(CCNCC2)CN1c1ccnc(Cl)n1] | 267.03 |
| | Method for Int. 34 | 2-(2-chloropyrimidin-4-yl)-2,8- | |
| | | diazaspiro[4.5]decan-3-one | |
| 71 | 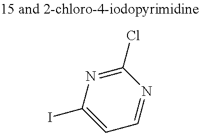 | 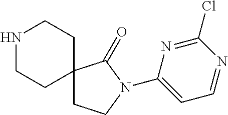 [SMILES: O=C1N(c2ccnc(Cl)n2)CCC12CCNCC2] | 267.00 |
| | Method for Int. 34 | 2-(2-chloropyrimidin-4-yl)-2,8- | |
| | | diazaspiro[4.5]decan-3-one | |
| 72 | 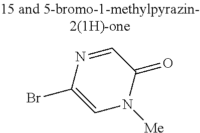 | 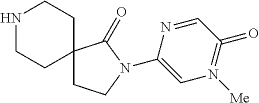 | 263.06 |
| | Method for Int. 44 | 2-(4-methyl-5-oxo-4,5- | |
| | | dihydropyrazin-2-yl)-2,8- | |
| | | diazaspiro[4.5]decan-1-one | |
| 73 | 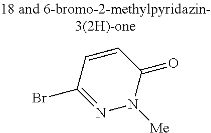 | 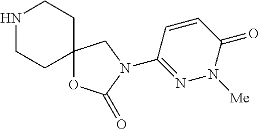 [SMILES: Cn1nc(N2CC3(CCNCC3)OC2=O)ccc1=O] | 264.96 |
| | Method for Int. 34 | 3-(1-methyl-6-oxo-1,6- | |
| | | dihydropyridazin-3-yl)-1-oxa-3,8- | |
| | | diazaspiro[4.5]decan-2-one | |
| 74 | 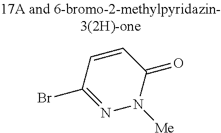 | 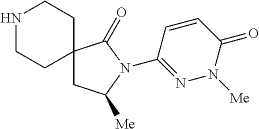 [SMILES: C[C@H]1CC2(CCNCC2)C(=O)N1c1ccc(=O)n(C)n1] | 277.19 |
| | Method for Int. 34 | (S)-3-methyl-2-(1-methyl-6-oxo-1,6- | |
| | | dihydropyridazin-3-yl)-2,8- | |
| | | diazaspiro[4.5]decan-1-one | |
| 75 | 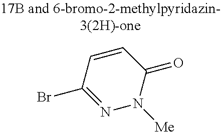 | 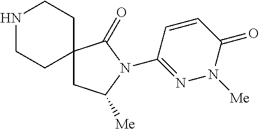 [SMILES: C[C@@H]1CC2(CCNCC2)C(=O)N1c1ccc(=O)n(C)n1] | 277.1 |
| | Method for Int. 34 | | |
| 76 | 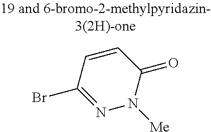 | 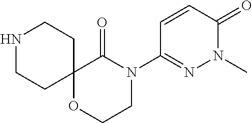 [SMILES: Cn1nc(N2CCOC3(CCNCC3)C2=O)ccc1=O] | 279 |
| | Method for Int. 34 | 4-(1-methyl-6-oxo-1,6- | |
| | | dihydropyridazin-3-yl)-1-oxa-4,9- | |
| | | diazaspiro[5.5]undecan-5-one | |

### Intermediate 77

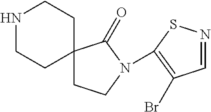 [SMILES: O=C1N(c2sncc2Br)CCC12CCNCC2]

### Step A: tert-butyl 2-(isothiazol-5-yl)-1-oxo-2,8-diazaspiro[4.5]decane-8-carboxylate

To a microwave vial was charged tert-butyl 1-oxo-2,8-diazaspiro[4.5]decane-8-carboxylate (INTERMEDIATE 15, 120 mg, 0.472 mmol), 5-bromoisothiazole (77 mg, 0.472 mmol), Pd₂(dba)₃ (8.64 mg, 9.44 μmol), Xantphos (16.4 mg, 0.028 mmol), and cesium carbonate (231 mg, 0.708 mmol). The vial was sealed, degased, and filled with dioxane (2.4 mL). The reaction mixture was heated at 95° C. overnight, and diluted with water, extracted with EtOAc. The organic layer was washed with brined, dried, evaporated to give the crude product, which was purified by silica gel column chromatography (0-100% EtOAc/hex as eluent) to afford the title compound. LC/MS: (M+1)⁺: 338.26.

### Step B: tert-butyl 2-(4-bromoisothiazol-5-yl)-1-oxo-2,8-diazaspiro[4.5]decane-8-carboxylate

To tert-butyl 2-(isothiazol-5-yl)-1-oxo-2,8-diazaspiro[4.5]decane-8-carboxylate (65 mg, 0.193 mmol) in DCM (1.9 mL) was added NBS (41.1 mg, 0.231 mmol). The reaction mixture was heated at 40° C. for 2 h, and diluted with water, extracted with EtOAc. The organic layer was washed with brined, dried, evaporated to give the crude product, which was purified by silica gel column chromatography (0-10% MeOH/DCM as eluent) to afford the title compound. LC/MS: (M+2)⁺: 417.99.

### Step C: 2-(4-bromoisothiazol-5-yl)-2,8-diazaspiro[4.5]decan-1-one

tert-Butyl 2-(4-bromoisothiazol-5-yl)-1-oxo-2,8-diazaspiro[4.5]decane-8-carboxylate (65 mg, 0.156 mmol) in DCM (1.6 mL) was treated with TFA (0.36 mL, 4.68 mmol) at 0° C. to free Boc protection and give TFA salt. The reaction mixture was concentrated, and treated with 0.1N NaOH aqueous solution, and extracted with IPA/CHCl₃ (1/3) to give the title compound as a free base after concentration. LC/MS: (M+1)⁺: 317.22.

### Intermediate 78

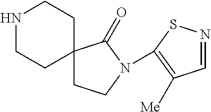 [SMILES: Cc1cnsc1N1CCC2(CCNCC2)C1=O]

### Step A: tert-butyl 2-(4-methylisothiazol-5-yl)-1-oxo-2,8-diazaspiro[4.5]decane-8-carboxylate

To a microwave vial was charged with tert-butyl 2-(4-bromoisothiazol-5-yl)-1-oxo-2,8-diazaspiro[4.5]decane-8-carboxylate (product of Step B of INTERMEDIATE 77, 50 mg, 0.120 mmol), 2,4,6-trimethyl-1,3,5,2,4,6-trioxatriborinane (60.3 mg, 0.240 mmol), Pd(dtbpf)Cl₂ (3.91 mg, 6.00 μmol), and potassium phosphate (102 mg, 0.480 mmol). DMF (1.1 mL) and water (0.1 mL) were added. The reaction mixture was degased and microwaved at 100° C. for 2 h. The reaction mixture was evaporated, and the crude product was purified by prep-TLC (2000 μm, 5% MeOH/DCM as eluent) to provide the title compound. LC/MS: (M+1)⁺: 352.20.

### Step B: 2-(pyridin-4-yl)-2,8-diazaspiro[4.5]decan-1-one

The title compound was prepared in an analogous fashion to Step C of INTERMEDIATE 77 starting from tert-butyl 2-(4-methylisothiazol-5-yl)-1-oxo-2,8-diazaspiro[4.5]decane-8-carboxylate. ¹H NMR (500 MHz, CDCl₃): δ 7.98 (s, 1H), 4.18 (t, J=7.0 Hz, 2H), 3.24-3.19 (m, 2H), 2.89-2.84 (m, 2H), 2.40 (s, 3H), 2.20 (t, J=7.0 Hz, 2H), 1.94-1.88 (m, 2H), 1.67-1.63 (m, 2H).

### Intermediate 79

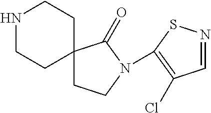 [SMILES: O=C1N(c2sncc2Cl)CCC12CCNCC2]

### 2-(4-chloroisothiazol-5-yl)-2,8-diazaspiro[4.5]decan-1-one

The title compound was prepared in an analogous fashion to INTERMEDIATE 77 starting from tert-butyl 2-(isothiazol-5-yl)-1-oxo-2,8-diazaspiro[4.5]decane-8-carboxylate and NCS. LC/MS: (M+1)⁺: 272.

### Intermediate 80

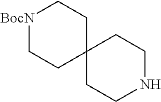 [SMILES: CC(C)(C)OC(=O)N1CCC2(CCNCC2)CC1]

### Step A: 9-benzyl-2,4-dioxo-3,9-diazaspiro[5.5]undecane-1,5-dicarbonitrile

A mixture of 1-benzylpiperidin-4-one (1 kg) and ethyl cyanoacetate (1.195 kg) in a saturated ethanolic ammonia solution (3 L) was stirred for 12 h at about 0-2° C. After completed detected by TLC, the reaction mixture was filtered and the solid was dried in vacuo to afford the title compound which was used for next step directly without further purification.

### Step B: diethyl 2,2′-(1-benzylpiperidine-4,4-diyl)diacetate

The crude 9-benzyl-2,4-dioxo-3,9-diazaspiro[5.5]undecane-1,5-dicarbonitrile in con H₂SO₄ (1.2 L) and water (1 L) was refluxed for 3 days until the starting material was consumed. The reaction mixture was neutralized by sodium carbonate (1.9 kg) and extracted with ethyl acetate. The combined organic layer was washed with brine, dried and concentrated in vacuo to afford the title compound.

### Step C: diethyl 2,2′-(1-(tert-butoxycarbonyl)piperidine-4,4-diyl)diacetate

A mixture of diethyl 2,2′-(1-benzylpiperidine-4,4-diyl)diacetate (500 g, 1.44 mol), Boc₂O (380 g) and Pd(OH)₂/C (50 g) in methanol (500 mL) under H₂ atmosphere (50 psi) was stirred for 24 hours at RT. The mixture was filtered and the filtrate was concentrated in vacuo to afford the title compound.

### Step D: tert-butyl 4,4-bis(2-hydroxyethyl)piperidine-1-carboxylate

To a suspension of LiAlH₄ (81.9 g, 2.15 mol) in dry THF (6 L) at −40° C. was added a solution of diethyl 2,2′-(1-(tert-butoxycarbonyl)piperidine-4,4-diyl)diacetate (478 g, 1.34 mol) in dry THF (2 L) for 2 hours, the reaction mixture was stirred for 0.5 h at this same temperature and warmed to RT slowly. Then the mixture was cooled to 0° C., water (85.8 mL), 1N sodium hydroxide solution (171.6 mL) and water (195 ml) was added slowly, the mixture was stirred for 0.5 h and filtered, washed with THF (150 mL×3). The filtrated was concentrated in vacuo to afford the title compound.

### Step E: tert-butyl 4,4-bis(2-((methylsulfonyl)oxy)ethyl)piperidine-1-carboxylate

To a solution of tert-butyl 4,4-bis(2-hydroxyethyl)piperidine-1-carboxylate (329 g, 1.21 mol) in dry DCM (3.5 L) at −25° C. was added TEA (505 mL, 3.62 mol) followed by addition of DMAP (32.9 g, 0.27 mol) and MsCl (310 g). The reaction mixture was stirred for 0.5 h at the same temperature. Then a solution of 10% citric acid was added, the organic layer was separated and the aqueous layer was extracted with ethyl acetate. The combined organic layer was washed with brine, dried and concentrated in vacuo to afford the title compound.

### Step F: tert-butyl 9-benzyl-3,9-diazaspiro[5.5]undecane-3-carboxylate

A mixture of tert-butyl 4,4-bis(2-((methylsulfonyl)oxy)ethyl)piperidine-1-carboxylate (500 g, 1.17 mol) and BnNH₂ (508 g, 4.75 mol) in ethanol (5 L) was refluxed for 20 h. The solvent was removed in vacuo, the residue was diluted with ethyl acetate and filtered to remove the salt. The filtrate was concentrated in vacuo and purified by column chromatography on silica gel (PE/EA=10/1) to afford the title compound.

### Step G: tert-butyl 3,9-diazaspiro[5.5]undecane-3-carboxylate

A mixture of tert-butyl 9-benzyl-3,9-diazaspiro[5.5]undecane-3-carboxylate (240 g, 0.7 mol) and Pd(OH)₂/C (24 g) in methanol (1.5 L) under hydrogen atmosphere (60 psi) at 40° C. for 24 h. The mixture was filtered and the filtrate was concentrated in vacuo. The residue was treated with 1N HCl/methanol and filtered to afford compound the title compound as the HCl salt. ¹H NMR (400 MHz, DMSO-d6) δ 8.91 (br s, 1H), 3.28 (t, J=4.8 Hz, 4H), 2.99 (t, J=5.6 Hz, 4H), 1.62 (t, J=6 Hz, 4H), 1.42-1.33 (m, 4H), 1.38 (s, 9H).

### Intermediate 81

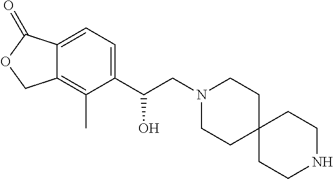

### Step A: (R)-tert-butyl 9-(2-hydroxy-2-(4-methyl-1-oxo-1,3-dihydroisobenzofuran-5-yl)ethyl)-3,9-diazaspiro[5.5]undecane-3-carboxylate

To a solution of tert-butyl 3,9-diazaspiro[5.5]undecane-3-carboxylate hydrochloride (114 g, 0.39 mol) in ethanol (1 L) was added Et₃N (60 mL). The mixture was stirred for 2 hours. Next, (R)-4-methyl-5-(oxiran-2-yl)isobenzofuran-1(3H)-one (75 g, 0.39 mol) was added. The mixture was heated to reflux for 24 h. The mixture was concentrated, washed with brine, dried over Na₂SO₄ and concentrated to provide the crude product. The crude product was purified by SFC separation to provide the title compound. ¹H NMR (400 MHz, CDCl₃) δ 7.80-7.75 (m, 2H), 5.23 (s, 2H), 5.14-5.11 (m, 1H), 3.39-3.36 (m, 5H), 3.18-3.10 (m, 1H), 3.05-2.99 (m, 1H), 2.82-2.72 (m, 2H), 2.60-2.35 (m, 4H), 2.25 (s, 3H), 1.83-1.80 (m, 1H), 1.65-1.55 (m, 3H), 1.50-1.35 (m, 18H.)

### Step B: (R)-5-(1-hydroxy-2-(3,9-diazaspiro[5.5]undecan-3-yl)ethyl)-4-methylisobenzofuran-1(3H)-one

To a solution of (R)-tert-butyl 9-(2-hydroxy-2-(4-methyl-1-oxo-1,3-dihydroisobenzofuran-5-yl)ethyl)-3,9-diazaspiro[5.5]undecane-3-carboxylate (83 g, 0.19 mol) in CH₂Cl₂ (1 L) was slowly added a 4 M HCl solution (300 mL). The mixture was stirred for 8 h. The mixture was filtered. The solid was washed with CH₂Cl₂, dried to give product as HCl salt. NMR showed there was DEA remained. The product was dissolved in methanol, NaHCO₃ was added. The mixture was stirred for 3 h. After filtration, the filtrate was concentrated, washed with ethyl acetate. The residue was concentrated in vacuo to give the title compound. ¹H NMR (400 MHz, CDCl₃) δ 7.79 (m, 2H), 5.30 (s, 1H), 5.25 (s, 2H), 5.07 (m, 1H), 4.03 (s, 1H), 3.14 (m, 4H), 2.76 (m, 2H), 2.55 (m, 1H), 2.41 (m, 3H), 2.26 (s, 3H), 1.80 (m, 4H), 1.65 (m, 4H).

### Intermediate 82

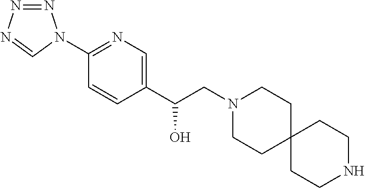

### Step A: (R)-tert-butyl 9-(2-(6-(1H-tetrazol-1-yl)pyridin-3-yl)-2-hydroxyethyl)-3,9-diazaspiro[5.5]undecane-3-carboxylate

To a solution of tert-butyl 3,9-diazaspiro[5.5]undecane-3-carboxylate (11.6 g, 0.04 mol) in EtOH (104 mL) was added TEA (6 mL). The mixture was stirred for 3 h at 25 degree. (R)-5-(oxiran-2-yl)-2-(1H-tetrazol-1-yl)pyridine (7.56 g, 0.04 mol) was added. The mixture was heated to reflux for 48 h., concentrated, washed with brines, dried over Na₂SO₄, and concentrated to provide the crude product. Further purified by chiral SFC (Column: AD, 250×30 mm I.D. 20 um; Mobile phase: A: supercritical CO₂, B: EtOH (0.05% NH₃.H₂O); flow rate: 80 mL/min; Back pressure: 100 bar; wavelength: 220 nm; column temperature: 38° C.) to afford the title compound. ¹H NMR (400 MHz, CDCl₃) δ 9.53 (s, 1H), 8.51 (d, J=1.6 Hz, 1H), 8.08-8.02 (m, 2H), 4.84 (dd, J₁=10.8 Hz, J₂=3.6 Hz, 1H), 3.38 (t, J=2.4 Hz, 4H), 2.74 (t, J=4.8 Hz, 2H), 2.61 (dd, J₁=12.4 Hz, J₂=3.6 Hz, 1H), 2.47-2.41 (m, 3H), 1.55 (dd, J₁=10.8 Hz, J₂=6.8 Hz, 4H), 1.42 (s, 13H).

### Step B: (R)-1-(6-(1H-tetrazol-1-yl)pyridin-3-yl)-2-(3,9-diazaspiro[5.5]undecan-3-yl)ethanol

To a solution of (R)-tert-butyl 9-(2-(6-(1H-tetrazol-1-yl)pyridin-3-yl)-2-hydroxyethyl)-3,9-diazaspiro[5.5]undecane-3-carboxylate (15 g, 0.032 mol) in CH₂Cl₂ (187 mL) was slowly added a 4 M HCl solution (57 mL). The mixture was stirred for 3 h. The mixture was filtered. The solid was washed with CH₂Cl₂, dried to give the title compound as the HCl salt. ¹H NMR (400 MHz, D₂O) δ 9.72 (s, 1H), 8.53 (d, J=1.6 Hz, 1H), 8.08 (dd, J₁=8.4 Hz, J₂=1.6 Hz, 1H), 7.97 (d, J=8.4 Hz, 1H), 5.31-5.27 (m, 1H), 3.62 (d, J=13.2 Hz, 1H), 3.45 (d, J=13.2 Hz, 1H), 3.35 (d, J=6.8 Hz, 2H), 3.23-3.10 (m, 6H), 1.97-1.61 (m, 8H).

### Intermediate 83

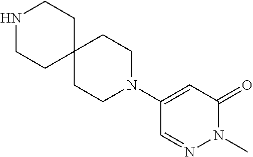 [SMILES: Cn1ncc(N2CCC3(CCNCC3)CC2)cc1=O]

### Step A: 5-iodo-2-methylpyridazin-3(2H)-one

5-Iodopyridazin-3(2H)-one (1000 mg, 4.50 mmol), potassium carbonate (1245 mg, 9.01 mmol), and anhydrous acetonitrile (22 mL) were charged to a roundbottom flask equipped with a rubber septum and magnetic stir bar. Methyl iodide (310 μL, 4.96 mmol) was charged dropwise via syringe. The flask was then equipped with a reflux condenser, heated at reflux for 3 hours. The reaction mixture was filtered through CELITE, and the filtrate containing the crude product was concentrated and purified on column chromatography (0-100% EtOAc:hex) to afford the title compound. ¹H NMR (500 MHz, CDCl₃) δ 7.91 (d, J=2.0 Hz, 1H), 7.44 (d, J=2.0 Hz, 1H), 3.70 (s, 3H).

### Step B: tert-butyl 9-(1-methyl-6-oxo-1,6-dihydropyridazin-4-yl)-3,9-diazaspiro[5.5]undecane-3-carboxylate

To a MW vial was charged tert-butyl 3,9-diazaspiro[5.5]undecane-3-carboxylate (220 mg, 0.756 mmol), 5-iodo-2-methylpyridazin-3(2H)-one (196 mg, 0.832 mmol), Pd₂(dba)₃ (17.32 mg, 0.019 mmol), Xantphos (32.8 mg, 0.057 mmol), and Cs₂CO₃ (493 mg, 1.513 mmol). The vial was sealed, degassed, and filled with anhydrous dioxane (2.5 mL). The reaction mixture was heated at 90° C. overnight, and filtered through CELITE®. The crude product was purified by column chromatography (0-10% MeOH/DCM) to afford the title compound. LC/MS: [(M+1)]⁺=363.

### Step C: 2-methyl-5-(3,9-diazaspiro[5.5]undecan-3-yl)pyridazin-3(2H)-one

Step C was conducted in a similar fashion to Step C of INTERMEDIATE 77. LC/MS: [(M+1)]⁺=263.

### Example 1

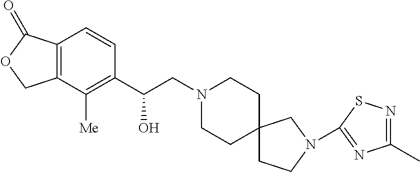 [SMILES: Cc1nsc(N2CCC3(CCN(C[C@H](O)c4ccc5c(c4C)COC5=O)CC3)C2)n1]

5-[(1R)-2-(2,8-diazaspiro[4.5]dec-8-yl)-1-hydroxyethyl]-4-methyl-2-benz ofuran-1(3H)-one (30 mg, 0.091 mmole), 5-chloro-3-methyl-1,2,4-thiadiazole (15 mg, 0.109 mmole) and diisopropyl ethylamine (0.048 ml, 0.272 mmole) were mixed in 0.5 ml of N,N-dimethylacetamide in a vial. The mixture was stirred at 80° C. overnight. The mixture was then cooled down to room temperature, diluted with 0.5 ml of DMSO and then purified by reverse phase mass directed HPLC system using acetonitrile and water to give 5-{(1R)-1-hydroxy-2-[2-(3-methyl-1,2,4-thiadiazol-5-yl)-2,8-diazaspiro[4.5]dec-8-yl]ethyl}-4-methyl-2-benzofuran-1(3H)-one. LC-MS (IE, m/z): 429.43 [M+1]⁺.

The compounds in Table 5 were prepared in an analogous fashion to EXAMPLE 1 starting from the amine intermediate and commercially available halides.

TABLE 5

| EX. | INT. | EXAMPLE STRUCTURE/NAME | LC/MS [M + 1]⁺ |
|---|---|---|---|
| 2 | 26 | 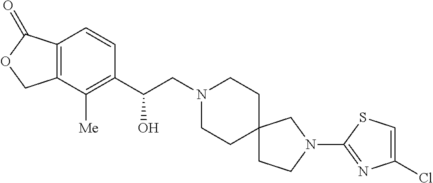 [SMILES: Cc1c([C@@H](O)CN2CCC3(CC2)CCN(c2nc(Cl)cs2)C3)ccc2c1COC2=O] | 448.37 |
| | | 5-{(1R)-2-[2-(4-chloro-1,3-thiazol-2-yl)-2,8- | |
| | | diazaspiro[4.5]dec-8-yl]-1-hydroxyethyl}-4-methyl-2- | |
| | | benzofuran-1(3H)-one | |
| 3 | 26 | 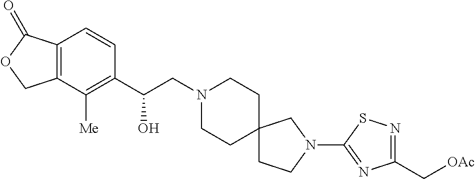 [SMILES: CC(=O)OCc1nsc(N2CCC3(CCN(C[C@H](O)c4ccc5c(c4C)COC5=O)CC3)C2)n1] | 487.41 |
| | | (5-{8-[(2R)-2-hydroxy-2-(4-methyl-1-oxo-1,3-dihydro-2- | |
| | | benzofuran-5-yl)ethyl]-2,8-diazaspiro[4.5]dec-2-yl}-1,2,4- | |
| | | thiadiazol-3-yl)methyl acetate | |
| 4 | 26 | 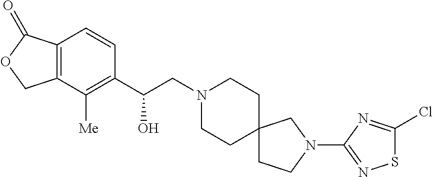 [SMILES: Cc1c([C@@H](O)CN2CCC3(CC2)CCN(c2nsc(Cl)n2)C3)ccc2c1COC2=O] | 449.2 |
| | | (R)-5-(2-(2-(5-chloro-1,2,4-thiadiazol-3-yl)-2,8- | |
| | | diazaspiro[4.5]decan-8-yl)-1-hydroxyethyl)-4- | |
| | | methylisobenzofuran-1(3H)-one | |
| 5 | 26 | 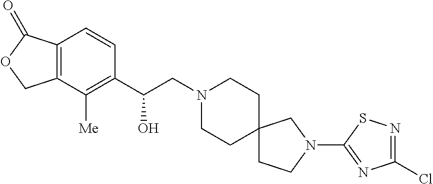 [SMILES: Cc1c([C@@H](O)CN2CCC3(CC2)CCN(c2nc(Cl)ns2)C3)ccc2c1COC2=O] | 450.1 |
| | | (R)-5-(2-(2-(3-chloro-1,2,4-thiadiazol-5-yl)-2,8- | |
| | | diazaspiro[4.5]decan-8-yl)-1-hydroxyethyl)-4- | |
| | | methylisobenzofuran-1(3H)-one | |
| 6 | 27 | 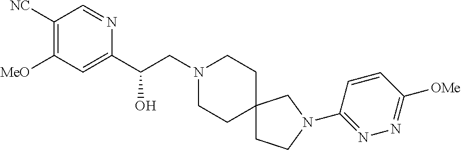 [SMILES: COc1ccc(N2CCC3(CCN(C[C@H](O)c4cc(OC)c(C#N)cn4)CC3)C2)nn1] | 425 |
| | | (S)-6-(1-hydroxy-2-(2-(6-methoxypyridazin-3-yl)-2,8- | |
| | | diazaspiro[4.5]decan-8-yl)ethyl)-4-methoxynicotinonitrile | |

### Example 7

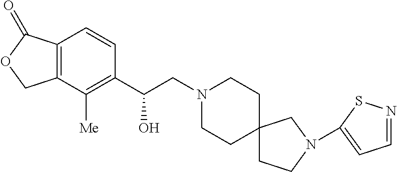 [SMILES: Cc1c([C@@H](O)CN2CCC3(CC2)CCN(c2ccns2)C3)ccc2c1COC2=O]

In a 1 dram vial were added (R)-8-(2-hydroxy-2-(4-methyl-1-oxo-1,3-dihydroisobenzofuran-5-yl)ethyl)-2,8-diazoniaspiro[4.5]decane chloride (52 mg, 0.13 mmol), cesium carbonate (101 mg, 0.311 mmol), tris(dibenzylideneacetone)dipalladium(0) (5 mg, 0.005 mmol), (9,9-dimethyl-9H-xanthene-4,5-diyl)bis(diphenylphosphine) (12 mg, 0.021 mmol), 5-bromoisothiazole (26 mg, 0.156 mmol) and 1 mL of degassed (nitrogen) DMA. The vial was purged with nitrogen gas, sealed and placed on a heating/stir block at 85° C. for 16 hours. The solutions from the reaction was filtered and the product purified by semi-preparative HPLC (gradient of 0-40% acetonitrile over 12 min.). The solvent was removed from the fractions containing the pure product (Genevac) and the resulting film was reconstituted in 1 mL of water/acetonitrile (9:1) and lyophilized to provide (R)-5-(1-hydroxy-2-(2-(isothiazol-5-yl)-2,8-diazaspiro[4.5]decan-8-yl)ethyl)-4-methylisobenzofuran-1(3H)-one. LC-MS (IE, m/z): 414.3 [M+1]⁺.

### Example 8

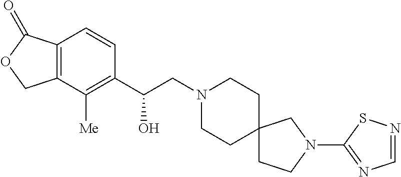 [SMILES: Cc1c([C@@H](O)CN2CCC3(CC2)CCN(c2ncns2)C3)ccc2c1COC2=O]

While working in an inert atmosphere glove box, to a 4 mL 1 dram containing 1 mL t-amyl alcohol, which had been degassed under nitrogen for 1 hour, was charged 5-bromo-1,2,4-thiadiazole (25 mg, 0.15 mmol), (R)-5-(1-hydroxy-2-(2,8-diazaspiro[4.5]decan-8-yl)ethyl)-4-methylisobenzofuran-1(3H)-one (0.040 g, 0.121 mmol), [dicyclohexyl(2′,6′-diisopropoxybiphenyl-2-yl)phosphine [RuPhos] (2.82 mg, 6.05 μmol) and {2-[2-(azanidyl-kN)ethyl]phenyl-kC¹}{[2′,6′-bis(propan-2-yloxy)biphenyl-2-yl](dicyclohexyl)phosphane-kP}chloropalladium(1+) [RuPhos-precatalyst] (4.94 mg, 6.05 μmol) and cesium carbonate (0.158 g, 0.484 mmol). The vial was fitted with a stir bar, capped and placed in a heating block at 95° C. with stirring for 16 hours. After 16 hours, the solvents were removed (Genevac), and the product was dissolved in 1.5 mL of DMSO, the solution was filtered and the product was purified by semi-preparative HPLC (gradient of 0-40% acetonitrile over 12 min.). The solvent was removed from the fractions containing the pure product (Genevac) and the resulting film was reconstituted in 1 mL of water/acetonitrile (9:1) and lyophilized to dryness to provide (R)-5-(2-(2-(1,2,4-thiadiazol-5-yl)-2,8-diazaspiro[4.5]decan-8-yl)-1-hydroxyethyl)-4-methylisobenzofuran-1(3H)-one. LC-MS (IE, m/z): 415.4 [M+1]⁺.

### Example 9

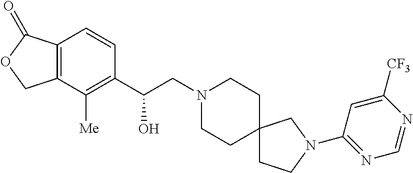 [SMILES: Cc1c([C@@H](O)CN2CCC3(CC2)CCN(c2cc(C(F)(F)F)ncn2)C3)ccc2c1COC2=O]

While working in an inert atmosphere glove box, to a 4 mL 1 dram vial containing 1 mL of 1,4-dioxane, which had been degassed under nitrogen for 1 hour, was charged (R)-5-(1-hydroxy-2-(2,8-diazaspiro[4.5]decan-8-yl)ethyl)-4-methylisobenzofuran-1(3H)-one (40 mg, 0.121 mmol), tris(dibenzylideneacetone)dipalladium(0) (5.5 mg, 0.006 mmol), (9,9-dimethyl-9H-xanthene-4,5-diyl)bis(diphenylphosphine) (14 mg, 0.024 mmol), 4-chloro-6-(trifluoromethyl)pyrimidine (27 mg, 0.15 mmol) and potassium tert-butoxide (1M in THF) (145 uL, 0.145 mmol). The vial was purged with nitrogen gas, sealed and placed on a heating/stir block at 85° C. for 16 hours. The solutions from the reaction was filtered and the product purified by semi-preparative HPLC (gradient of 0-40% acetonitrile over 12 min). The solvent was removed from the fractions containing the pure product (Genevac) and the resulting film was reconstituted in 1 mL of water/acetonitrile (9:1) and lyophilized to dryness to provide (R)-5-(1-hydroxy-2-(2-(6-(trifluoromethyl)pyrimidin-4-yl)-2, 8-diazaspiro[4.5]decan-8-yl)ethyl)-4-methylisobenzofuran-1(3H)-one. LC-MS (IE, m/z): 477.5 [M+1]⁺.

### Example 10

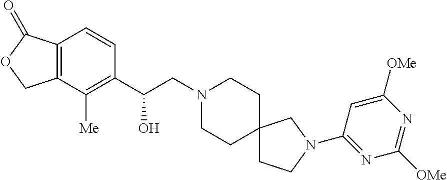 [SMILES: COc1cc(N2CCC3(CCN(C[C@H](O)c4ccc5c(c4C)COC5=O)CC3)C2)nc(OC)n1]

To a microwave vial was charged (R)-5-(1-hydroxy-2-(2,8-diazaspiro[4.5]decan-8-yl)ethyl)-4-methylisobenzofuran-1(3H)-one (40 mg, 0.121 mmol), 4-bromo-2,6-dimethoxypyrimidine (26.5 mg, 0.121 mmol), tris(dibenzylideneacetone)dipalladium(0) (5.54 mg, 6.05 μmol), 2-dicyclohexylphosphino-2′,4′,6′-triisopropylbiphenyl (11.5 mg, 0.024 mmol), and potassium phosphate (51.4 mg, 0.242 mmol). The vial was sealed, degased, and filled with dioxane (0.60 mL). The reaction mixture was heated at 100° C. overnight, diluted with water, extracted with EtOAc. The organic layer was washed with brined, dried, evaporated to give the crude product, which was purified by silica gel column chromatography (0-10% MeOH/DCM) to afford (R)-5-(2-(2-(2,6-dimethoxypyrimidin-4-yl)-2,8-diazaspiro[4.5]decan-8-yl)-1-hydroxyethyl)-4-methylisobenzofuran-1(3H)-one. LC-MS (ESI, m/z): 469 [M+1]⁺.

The compounds in Table 6 were prepared in an analogous fashion to EXAMPLES 7-10 starting from the indicated amine intermediate and commercially available halides.

TABLE 6

| EX. | INT. | EXAMPLE STRUCTURE/NAME | LC/MS [M + 1]⁺ |
|---|---|---|---|
| 11 | 26, method for Ex. 8 | 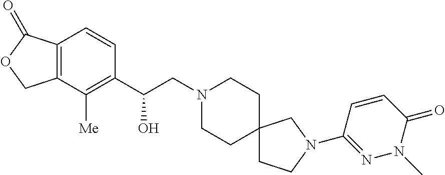 [SMILES: Cc1c([C@@H](O)CN2CCC3(CC2)CCN(c2ccc(=O)n(C)n2)C3)ccc2c1COC2=O] | 439.5 |
| | | (R)-6-(8-(2-hydroxy-2-(4-methyl-1-oxo-1,3- | |
| | | dihydroisobenzofuran-5-yl)ethyl)-2,8-diazaspiro[4.5]decan-2- | |
| | | yl)-2-methylpyridazin-3(2H)-one | |
| 12 | 26, method for Ex. 9 | 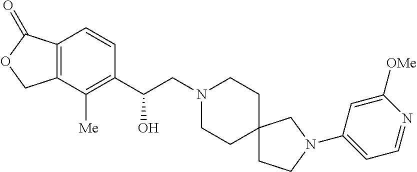 [SMILES: COc1cc(N2CCC3(CCN(C[C@H](O)c4ccc5c(c4C)COC5=O)CC3)C2)ccn1] | 438.5 |
| | | (R)-5-(1-hydroxy-2-(2-(2-methoxypyridin-4-yl)-2,8- | |
| | | diazaspiro[4.5]decan-8-yl)ethyl)-4-methylisobenzofuran- | |
| | | 1(3H)-one | |

### Example 13

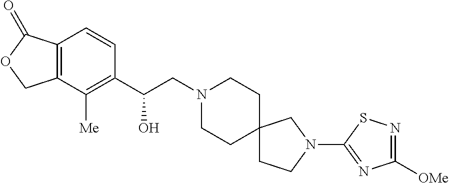 [SMILES: COc1nsc(N2CCC3(CCN(C[C@H](O)c4ccc5c(c4C)COC5=O)CC3)C2)n1]

A microwave vial was charged with (R)-4-methyl-5-(oxiran-2-yl)isobenzofuran-1(3H)-one (97 mg, 0.510 mmol), 3-methoxy-5-(2,8-diazaspiro[4.5]decan-2-yl)-1,2,4-thiadiazole (108 mg, 0.425 mmol), and ethanol (1.5 mL). The vial was sealed and heated via a microwave at 145° C. for 35 min. The solvent was evaporated and the residue was purified by prep-TLC (silica, 1000 μm, 7% MeOH:EtOAc as eluent) to afford (R)-5-(1-hydroxy-2-(2-(3-methoxy-1,2,4-thiadiazol-5-yl)-2,8-diazaspiro[4.5]decan-8-yl)ethyl)-4-methylisobenzofuran-1(3H)-one. LC/MS: [(M+1)]⁺=445.

The compounds In Table 7 were prepared in an analogous fashion to EXAMPLE 13 starting from epoxide and amine intermediates specified below.

TABLE 7

| EX. | INT. | EXAMPLE STRUCTURE/NAME | LC/MS [M + 1]⁺ |
|---|---|---|---|
| 14 | 4A, 30 | 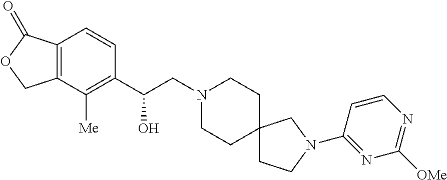 [SMILES: COc1nccc(N2CCC3(CCN(C[C@H](O)c4ccc5c(c4C)COC5=O)CC3)C2)n1] | 439 |
| | | (R)-5-(1-hydroxy-2-(2-(2-methoxypyrimidin-4-yl)-2,8- | |
| | | diazaspiro[4.5]decan-8-yl)ethyl)-4-methylisobenzofuran- | |
| | | 1(3H)-one | |
| 15 | 4A, 31 | 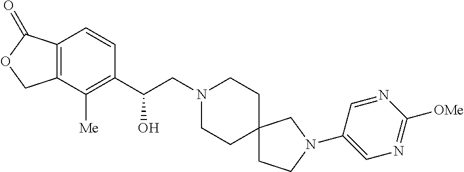 [SMILES: COc1ncc(N2CCC3(CCN(C[C@H](O)c4ccc5c(c4C)COC5=O)CC3)C2)cn1] | 439 |
| | | (R)-5-(1-hydroxy-2-(2-(2-methoxypyrimidin-5-yl)-2,8- | |
| | | diazaspiro[4.5]decan-8-yl)ethyl)-4-methylisobenzofuran- | |
| | | 1(3H)-one | |
| 16 | 4A, 32 | 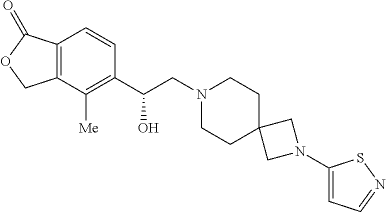 [SMILES: Cc1c([C@@H](O)CN2CCC3(CC2)CN(c2ccns2)C3)ccc2c1COC2=O] | 400 |
| | | (R)-5-(1-hydroxy-2-(2-(isothiazol-5-yl)-2,7- | |
| | | diazaspiro[3.5]decan-7-yl)ethyl)-4-methylisobenzofuran- | |
| | | 1(3H)-one | |
| 17 | 4A, 33 | 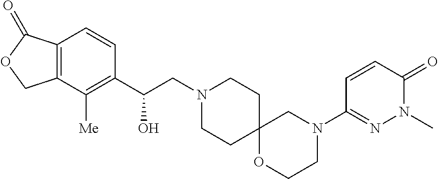 [SMILES: Cc1c([C@@H](O)CN2CCC3(CC2)CN(c2ccc(=O)n(C)n2)CCO3)ccc2c1COC2=O] | 455 |
| | | (R)-6-(9-(2-hydroxy-2-(4-methyl-1-oxo-1,3- | |
| | | dihydroisobenzofuran-5-yl)ethyl)-1-oxa-4,9- | |
| | | diazaspiro[5.5]undecan-4-yl)-2-methylpyridazin-3(2H)-one | |
| 18 | 10A, 33 | 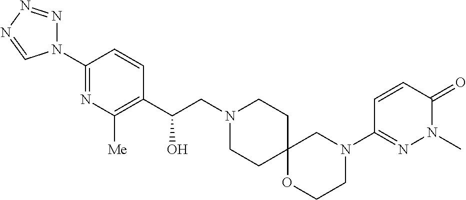 [SMILES: Cc1nc(-n2cnnn2)ccc1[C@@H](O)CN1CCC2(CC1)CN(c1ccc(=O)n(C)n1)CCO2] | 468 |
| | | (R)-6-(9-(2-hydroxy-2-(2-methyl-6-(1H-tetrazol-1-yl)pyridin- | |
| | | 3-yl)ethyl)-1-oxa-4,9-diazaspiro[5.5]undecan-4-yl)-2- | |
| | | methylpyridazin-3(2H)-one | |
| 19 | 11A, 33 | 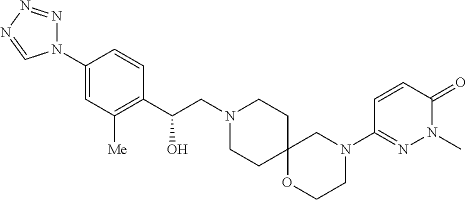 [SMILES: Cc1cc(-n2cnnn2)ccc1[C@@H](O)CN1CCC2(CC1)CN(c1ccc(=O)n(C)n1)CCO2] | 467 |
| | | (R)-6-(9-(2-hydroxy-2-(2-methyl-4-(1H-tetrazol-1- | |
| | | yl)phenyl)ethyl)-1-oxa-4,9-diazaspiro[5.5]undecan-4-yl)-2- | |
| | | methylpyridazin-3(2H)-one | |

### Example 20

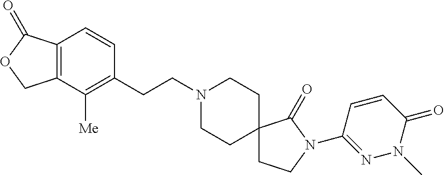 [SMILES: Cc1c(CCN2CCC3(CC2)CCN(c2ccc(=O)n(C)n2)C3=O)ccc2c1COC2=O]

To a microwave vial was added 5-ethenyl-4-methyl-2-benzofuran-1(3H)-one (133 mg, 0.762 mmol), 2-(1-methyl-6-oxo-1,6-dihydropyridazin-3-yl)-2,8-diazaspiro[4.5]decan-1-one (INTERMEDIATE 34, 50 mg, 0.191 mmol), bis(2-diphenylphosphinophenyl)ether (10.3 mg, 0.019 mmol) and bis(1,5-cyclooctadiene)rhodium(I) tetrafluoroborate (7.74 mg, 0.019 mmol). The vial was sealed, evacuated, and back filled with N₂ (repeat this twice). Toluene (0.5 mL) was added. The reaction mixture was heated at 70° C. for 48 h, filtered through CELITE®, rinsed with DCM, and the crude product was purifed with silica gel column chromatography (0-10% MeOH/DCM) to afford 8-(2-(4-methyl-1-oxo-1,3-dihydroisobenzofuran-5-yl)ethyl)-2-(1-methyl-6-oxo-1,6-dihydropyridazin-3-yl)-2,8-diazaspiro[4.5]decan-1-one. LC/MS: 437M+1]⁺.

### Example 21

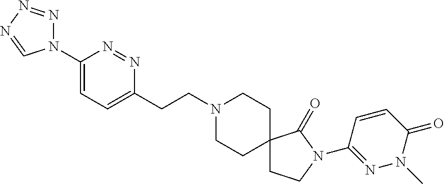 [SMILES: Cn1nc(N2CCC3(CCN(CCc4ccc(-n5cnnn5)nn4)CC3)C2=O)ccc1=O]

8-(2-(6-(1H-tetrazol-1-yl)pyridazin-3-yl)ethyl)-2-(1-methyl-6-oxo-1,6-dihydropyridazin-3-yl)-2,8-diazaspiro[4.5]decan-1-one was prepared from 3-(1H-tetrazol-1-yl)-6-vinylpyridazine (INTERMEDIATE 13) and 2-(1-methyl-6-oxo-1,6-dihydropyridazin-3-yl)-2,8-diazaspiro[4.5]decan-1-one (INTERMEDIATE 34) following the same procedure as described above for Example 20. LC-MS: 408.8 [M+1-28]⁺.

### Example 22

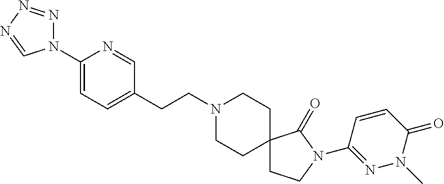 [SMILES: Cn1nc(N2CCC3(CCN(CCc4ccc(-n5cnnn5)nc4)CC3)C2=O)ccc1=O]

To 2-(1-methyl-6-oxo-1,6-dihydropyridazin-3-yl)-2,8-diazaspiro[4.5]decan-1-one (30 mg, 0.114 mmol) and 2-(6-(1H-tetrazol-1-yl)pyridin-3-yl)acetaldehyde (32.5 mg, 0.172 mmol) in THF (4.6 mL) was added acetic acid (39 μL, 0.686 mmol) and MP-cyanoborohydride (2.19 mmol/g) (172 mg, 0.377 mmol). The reaction mixture was put on shaker overnight, filtered, and rinsed with MeOH. The filtrate was concentrated, and purified by prep-TLC (5% MeOH/DCM) to give 8-(2-(6-(1H-tetrazol-1-yl)pyridin-3-yl)ethyl)-2-(1-methyl-6-oxo-1,6-dihydropyridazin-3-yl)-2,8-diazaspiro[4.5]decan-1-one. LC/MS: [(M+1-28)]⁺=408.0.

### Example 23

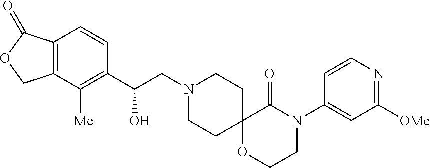 [SMILES: COc1cc(N2CCOC3(CCN(C[C@H](O)c4ccc5c(c4C)COC5=O)CC3)C2=O)ccn1]

(R)-9-(2-Hydroxy-2-(4-methyl-1-oxo-1,3-dihydroisobenzofuran-5-yl)ethyl)-1-oxa-4,9-diazaspiro[5.5]undecan-5-one (180 mg, 0.50 mmol) and 4-iodo-2-methoxypyridine (129 mg, 0.55 mmol), CuI (96 mg, 0.50 mmol), K₃PO₄ (106 mg, 0.50 mmol), (1R,2R)—N1,N2-dimethylcyclohexane-1,2-diamine (71 mg, 0.50 mmol) in toluene (6 mL) was stirred at 110° C. for 20 hours. The mixture was concentrated and the residue was purified by column chromatography to afford (R)-9-(2-hydroxy-2-(4-methyl-1-oxo-1,3-dihydroisobenzofuran-5-yl)ethyl)-4-(2-methoxypyridin-4-yl)-1-oxa-4,9-diazaspiro[5.5]undecan-5-one. LC-MS (ESI, m/z): 468 [M+1]⁺.

### Example 24

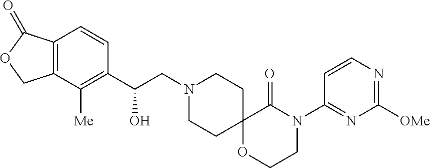 [SMILES: COc1nccc(N2CCOC3(CCN(C[C@H](O)c4ccc5c(c4C)COC5=O)CC3)C2=O)n1]

The mixture of (R)-9-(2-hydroxy-2-(4-methyl-1-oxo-1,3-dihydroisobenzofuran-5-yl)ethyl)-1-oxa-4,9-diazaspiro[5.5]undecan-5-one (400 mg, 1.11 mmol) and 4-bromo-2-methoxypyrimidine (420 mg, 2.22 mmol), Xantphos (40 mg, 10%), Pd₂(dba)₃ (40 mg, 10%), Cs₂CO₃ (545 mg, 1.67 mmol) in toluene (6 mL) was stirred at 110° C. for 20 hours. The mixture was concentrated and the residue was purified by prep-TLC (DCM/MeOH: 10/1) to afford (R)-9-(2-hydroxy-2-(4-methyl-1-oxo-1,3-dihydroisobenzofuran-5-yl)ethyl)-4-(2-methoxypyrimidin-4-yl)-1-oxa-4,9-diazaspiro[5.5]undecan-5-one. LC-MS (ESI, m/z): 469 [M+1]⁺.

### Example 25

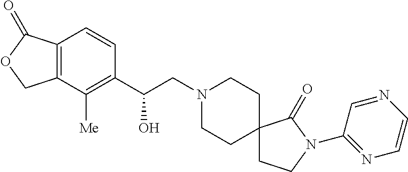 [SMILES: Cc1c([C@@H](O)CN2CCC3(CC2)CCN(c2cnccn2)C3=O)ccc2c1COC2=O]

To a mixture of (R)-8-(2-hydroxy-2-(4-methyl-1-oxo-1,3-dihydroisobenzofuran-5-yl)ethyl)-2,8-diazaspiro[4.5]decan-1-one (30 mg, 0.087 mmol), 2-chloropyrazine (20 mg, 0.174 mmol), RuPhos pre-catalyst (3.2 mg, 4.4 μmol) and RuPhos (4.1 mg, 8.8 μmol) was added anhydrous 2-methyl-2-butanol (1 ml) in a glove box. Then solid Cs₂CO₃ (85 mg, 0.261 mmol) was added. This reaction mixture was heated at 80° C. for 16 hr and then cooled to ambient temperature. The solution was filtrated and the solvent was evaporated under reduced pressure. The residue was dissolved in DMSO (1.5 mL) and the solution was filtered. The crude product was purified by reversed-phase HPLC (acetonitrile with 0.1% TFA: water with 0.1% TFA from 10% to 60%) to give (R)-8-(2-hydroxy-2-(4-methyl-1-oxo-1,3-dihydroisobenzofuran-5-yl)ethyl)-2-(pyrazin-2-yl)-2,8-diazaspiro[4.5]decan-1-one. LC-MS (IE, m/z): 423 [M+1]⁺.

The compounds in Table 8 were prepared in an analogous fashion to EXAMPLE 23-25 starting from spirolactam prepared as described above and other commercially available intermediates.

TABLE 8

| | | | LC/MS |
|---|---|---|---|
| EX. | INT. | EXAMPLE STRUCTURE/NAME | [M + 1]⁺ |
| 26 | 23 and 4- iodopyridine, method for Ex. 25 | 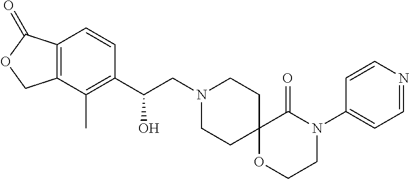 [SMILES: Cc1c([C@@H](O)CN2CCC3(CC2)OCCN(c2ccncc2)C3=O)ccc2c1COC2=O] (R)-9-(2-hydroxy-2-(4-methyl-1-oxo-1,3-dihydroisobenzofuran-5- yl)ethyl)-4-(pyridin-4-yl)-1-oxa-4,9-diazaspiro[5.5]undecan-5-one | 438 |
| 27 | 23 and 28, method for Ex. 25 | 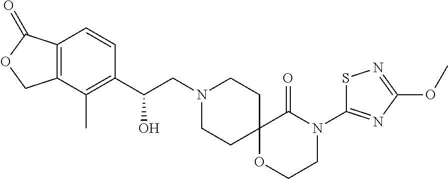 [SMILES: COc1nsc(N2CCOC3(CCN(C[C@H](O)c4ccc5c(c4C)COC5=O)CC3)C2=O)n1] (R)-9-(2-hydroxy-2-(4-methyl-1-oxo-1,3-dihydroisobenzofuran-5- yl)ethyl)-4-(3-methoxy-1,2,4-thiadiazol-5-yl)-1-oxa-4,9- diazaspiro[5.5]undecan-5-one | 478 |
| 28 | 23 and 35, method for Ex. 25 | 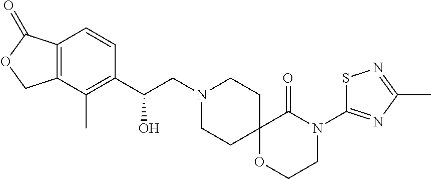 [SMILES: Cc1nsc(N2CCOC3(CCN(C[C@H](O)c4ccc5c(c4C)COC5=O)CC3)C2=O)n1] (R)-9-(2-hydroxy-2-(4-methyl-1-oxo-1,3-dihydroisobenzofuran-5- yl)ethyl)-4-(3-methyl-1,2,4-thiadiazol-5-yl)-1-oxa-4,9- diazaspiro[5.5]undecan-5-one | 459 |
| 29 | 24 and 6- bromo-2- methyl- pyridazin- 3(2H)- one, method for Ex. 25 | 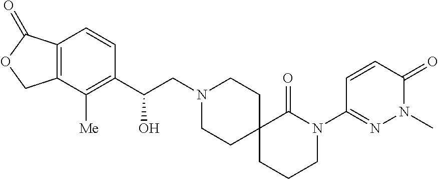 [SMILES: Cc1c([C@@H](O)CN2CCC3(CCCN(c4ccc(=O)n(C)n4)C3=O)CC2)ccc2c1COC2=O] (R)-9-(2-hydroxy-2-(4-methyl-1-oxo-1,3-dihydroisobenzofuran-5- yl)ethyl)-2-(1-methyl-6-oxo-1,6-dihydropyridazin-3-yl)-2,9- diazaspiro[5.5]undecan-1-one | 467.05 |
| 30 | 25 and 6- bromo-2- methyl- pyridazin- 3(2H)- one, method for Ex. 24 | 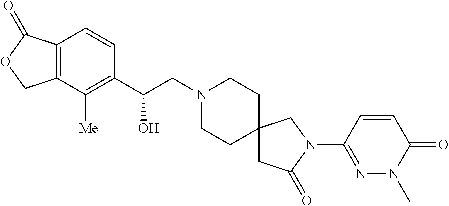 [SMILES: Cc1c([C@@H](O)CN2CCC3(CC2)CC(=O)N(c2ccc(=O)n(C)n2)C3)ccc2c1COC2=O] (R)-8-(2-hydroxy-2-(4-methyl-1-oxo-1,3-dihydroisobenzofuran-5- yl)ethyl)-2-(1-methyl-6-oxo-1,6-dihydropyridazin-3-yl)-2,8- diazaspiro[4.5]decan-3-one | 453.03 |
| 31 | 25 and 4- bromo-2- methoxy- pyrimidine, method for Ex. 24 | 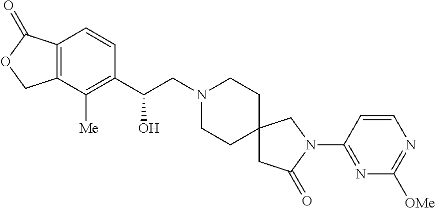 [SMILES: COc1nccc(N2CC3(CCN(C[C@H](O)c4ccc5c(c4C)COC5=O)CC3)CC2=O)n1] (R)-8-(2-hydroxy-2-(4-methyl-1-oxo-1,3-dihydroisobenzofuran-5- yl)ethyl)-2-(2-methoxypyrimidin-4-yl)-2,8-diazaspiro[4.5]decan- 3-one | 453.09 |
| 32 | 22 and 1- bromo-4- (trifluoromethoxy) benzene, method for Ex. 25 | 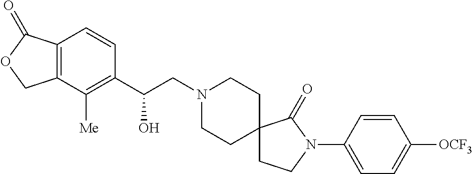 [SMILES: Cc1c([C@@H](O)CN2CCC3(CC2)CCN(c2ccc(OC(F)(F)F)cc2)C3=O)ccc2c1COC2=O] (R)-8-(2-hydroxy-2-(4-methyl-1-oxo-1,3-dihydroisobenzofuran-5- yl)ethyl)-2-(4-(trifluoromethoxy)phenyl)-2,8- diazaspiro[4.5]decan-1-one | 503.1 |
| 33 | 22 and 5- bromo-2- (difluoromethyl) pyridine, method for Ex. 25 | 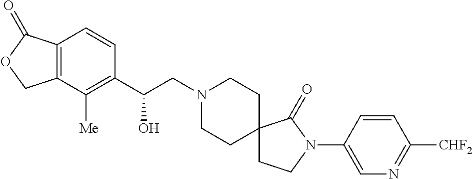 [SMILES: Cc1c([C@@H](O)CN2CCC3(CC2)CCN(c2ccc(C(F)F)nc2)C3=O)ccc2c1COC2=O] (R)-2-(6-(difluoromethyl)pyridin-3-yl)-8-(2-hydroxy-2-(4-methyl- 1-oxo-1,3-dihydroisobenzofuran-5-yl)ethyl)-2,8- diazaspiro[4.5]decan-1-one | 472.2 |
| 34 | 22 and 5- bromo-3- phenylisoxazole, method for Ex. 25 | 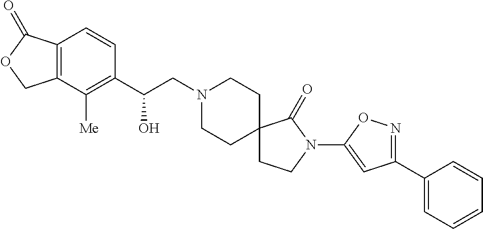 [SMILES: Cc1c([C@@H](O)CN2CCC3(CC2)CCN(c2cc(-c4ccccc4)no2)C3=O)ccc2c1COC2=O] (R)-8-(2-hydroxy-2-(4-methyl-1-oxo-1,3-dihydroisobenzofuran-5- yl)ethyl)-2-(3-phenylisoxazol-5-yl)-2,8-diazaspiro[4.5]decan-1- one | 488.4 |
| 35 | 22 and 3- chloropyridine, method for Ex. 26 | 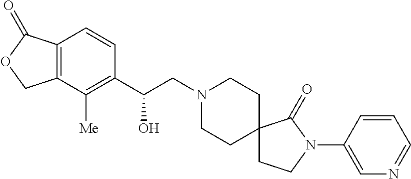 [SMILES: Cc1c([C@@H](O)CN2CCC3(CC2)CCN(c2cccnc2)C3=O)ccc2c1COC2=O] (R)-8-(2-hydroxy-2-(4-methyl-1-oxo-1,3-dihydroisobenzofuran-5- yl)ethyl)-2-(pyridin-3-yl)-2,8-diazaspiro[4.5]decan-1-one | 422 |
| 36 | 22 and 3- chloro-6- methoxypyridazine, method for Ex. 26 | 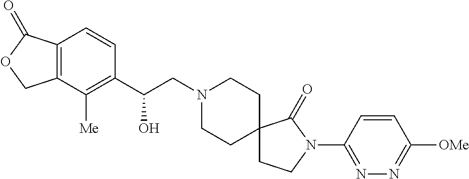 [SMILES: COc1ccc(N2CCC3(CCN(C[C@H](O)c4ccc5c(c4C)COC5=O)CC3)C2=O)nn1] (R)-8-(2-hydroxy-2-(4-methyl-1-oxo-1,3-dihydroisobenzofuran-5- yl)ethyl)-2-(6-methoxypyridazin-3-yl)-2,8-diazaspiro[4.5]decan- 1-one | 453 |
| 37 | 22 and 5- chloropyrimidine, method for Ex. 26 | 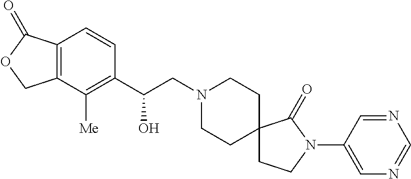 [SMILES: Cc1c([C@@H](O)CN2CCC3(CC2)CCN(c2cncnc2)C3=O)ccc2c1COC2=O] (R)-8-(2-hydroxy-2-(4-methyl-1-oxo-1,3-dihydroisobenzofuran-5- yl)ethyl)-2-(pyrimidin-5-yl)-2,8-diazaspiro[4.5]decan-1-one | 423 |
| 38 | 22 and 3- chloro-5- fluoropyridine, method for Ex. 26 | 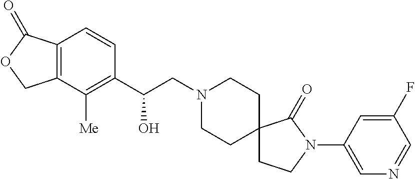 [SMILES: Cc1c([C@@H](O)CN2CCC3(CC2)CCN(c2cncc(F)c2)C3=O)ccc2c1COC2=O] (R)-2-(5-fluoropyridin-3-yl)-8-(2-hydroxy-2-(4-methyl-1-oxo-1,3- dihydroisobenzofuran-5-yl)ethyl)-2,8-diazaspiro[4.5]decan-1-one | 440 |
| 39 | 22 and 4- chloro-2- methoxypyrimidine (methoxy-d₃), method for Ex. 25 | 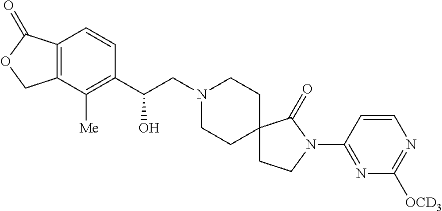 (R)-8-(2-hydroxy-2-(4-methyl-1-oxo-1,3-dihydroisobenzofuran-5- yl)ethyl)-2-(2-methoxypyrimidin-4-yl)-2,8-diazaspiro[4.5]decan- 1-one (methoxy-d₃) | 456 |
| 40 | 22 and 5- bromo-2- methoxypyrimidine, method for Ex. 24 | 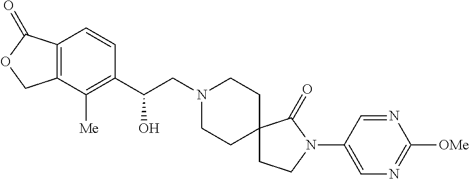 [SMILES: COc1ncc(N2CCC3(CCN(C[C@H](O)c4ccc5c(c4C)COC5=O)CC3)C2=O)cn1] (R)-8-(2-hydroxy-2-(4-methyl-1-oxo-1,3-dihydroisobenzofuran-5- yl)ethyl)-2-(2-methoxypyrimidin-5-yl)-2,8-diazaspiro[4.5]decan- 1-one | 453.03 |
| 41 | 22 and 4- bromo-2- methylpyrimidine, method for Ex. 24 | 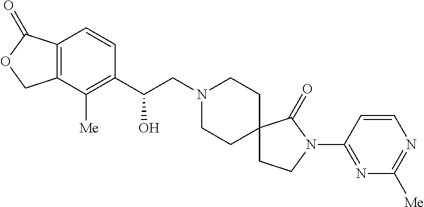 [SMILES: Cc1nccc(N2CCC3(CCN(C[C@H](O)c4ccc5c(c4C)COC5=O)CC3)C2=O)n1] (R)-8-(2-hydroxy-2-(4-methyl-1-oxo-1,3-dihydroisobenzofuran-5- yl)ethyl)-2-(2-methylpyrimidin-4-yl)-2,8-diazaspiro[4.5]decan-1- one | 437.07 |
| 42 | 22 and 4- bromo- 2,6- dimethoxypyrimidine, method for Ex. 24 | 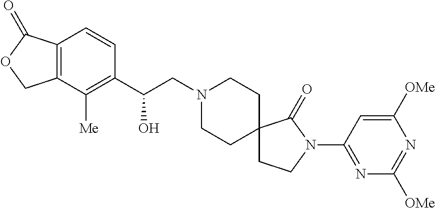 [SMILES: COc1cc(N2CCC3(CCN(C[C@H](O)c4ccc5c(c4C)COC5=O)CC3)C2=O)nc(OC)n1] (R)-2-(2,6-dimethoxypyrimidin-4-yl)-8-(2-hydroxy-2-(4-methyl- 1-oxo-1,3-dihydroisobenzofuran-5-yl)ethyl)-2,8- diazaspiro[4.5]decan-1-one | 483.10 |
| 43 | 22 and 4- bromo- N,N- dimethylpyrimidin- 2-amine, method for Ex. 24 | 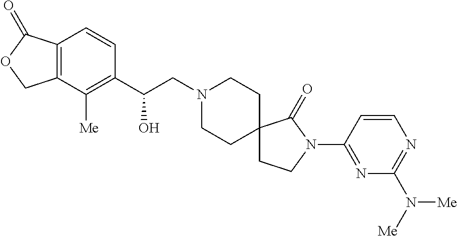 [SMILES: Cc1c([C@@H](O)CN2CCC3(CC2)CCN(c2ccnc(N(C)C)n2)C3=O)ccc2c1COC2=O] (R)-2-(2-(dimethylamino)pyrimidin-4-yl)-8-(2-hydroxy-2-(4- methyl-1-oxo-1,3-dihydroisobenzofuran-5-yl)ethyl)-2,8- diazaspiro[4.5]decan-1-one | 466.08 |
| 44 | 22 and 4- chloro-2- ethoxypyrimidine, method for Ex. 25 | 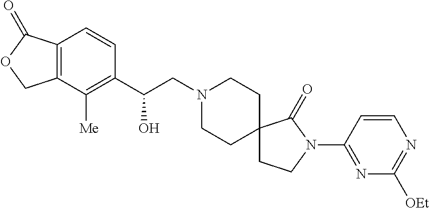 [SMILES: CCOc1nccc(N2CCC3(CCN(C[C@H](O)c4ccc5c(c4C)COC5=O)CC3)C2=O)n1] (R)-2-(2-ethoxypyrimidin-4-yl)-8-(2-hydroxy-2-(4-methyl-1-oxo- 1,3-dihydroisobenzofuran-5-yl)ethyl)-2,8-diazaspiro[4.5]decan-1- one | 467.03 |
| 45 | 22 and 4- bromo-2- ethylpyrimidine, method for Ex. 25 | 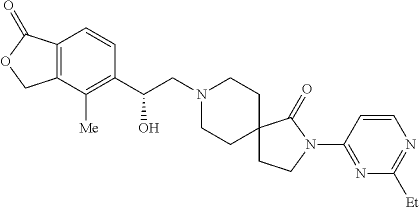 [SMILES: CCc1nccc(N2CCC3(CCN(C[C@H](O)c4ccc5c(c4C)COC5=O)CC3)C2=O)n1] (R)-2-(2-ethylpyrimidin-4-yl)-8-(2-hydroxy-2-(4-methyl-1-oxo- 1,3-dihydroisobenzofuran-5-yl)ethyl)-2,8-diazaspiro[4.5]decan-1- one | 451.11 |
| 46 | 22 and 4- bromopyrimidin- 2-amine, method for Ex. 24 | 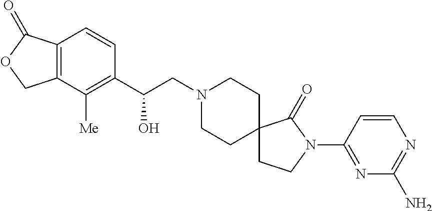 [SMILES: Cc1c([C@@H](O)CN2CCC3(CC2)CCN(c2ccnc(N)n2)C3=O)ccc2c1COC2=O] (R)-2-(2-aminopyrimidin-4-yl)-8-(2-hydroxy-2-(4-methyl-1-oxo- 1,3-dihydroisobenzofuran-5-yl)ethyl)-2,8-diazaspiro[4.5]decan-1- one | 438.02 |
| 47 | 22 and 4- bromo-N- methylpyrimidin- 2-amine, method for Ex. 24 | 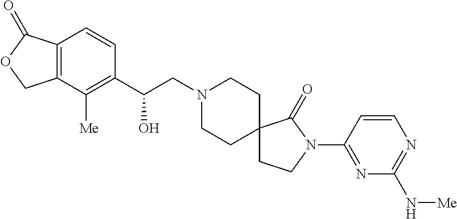 [SMILES: CNc1nccc(N2CCC3(CCN(C[C@H](O)c4ccc5c(c4C)COC5=O)CC3)C2=O)n1] (R)-8-(2-hydroxy-2-(4-methyl-1-oxo-1,3-dihydroisobenzofuran-5- yl)ethyl)-2-(2-(methylamino)pyrimidin-4-yl)-2,8- diazaspiro[4.5]decan-1-one | 452.06 |
| 48 | 22 and 4- chloro-2- (furan-3- yl) pyrimidine, method for Ex. 25 | 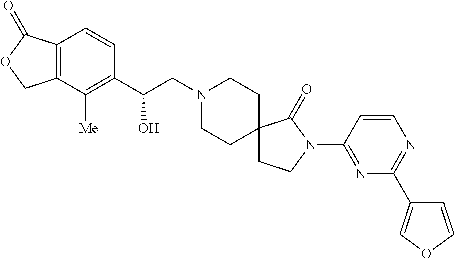 [SMILES: Cc1c([C@@H](O)CN2CCC3(CC2)CCN(c2ccnc(-c4ccoc4)n2)C3=O)ccc2c1COC2=O] (R)-2-(2-(furan-3-yl)pyrimidin-4-yl)-8-(2-hydroxy-2-(4-methyl-1- oxo-1,3-dihydroisobenzofuran-5-yl)ethyl)-2,8- diazaspiro[4.5]decan-1-one | 489.11 |
| 49 | 22 and 40, method for Ex. 25 | 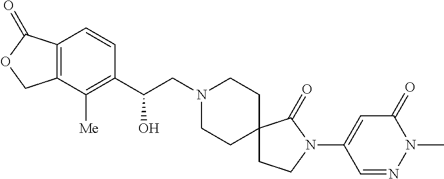 [SMILES: Cc1c([C@@H](O)CN2CCC3(CC2)CCN(c2cnn(C)c(=O)c2)C3=O)ccc2c1COC2=O] (R)-8-(2-hydroxy-2-(4-methyl-1-oxo-1,3-dihydroisobenzofuran-5- yl)ethyl)-2-(1-methyl-6-oxo-1,6-dihydropyridazin-4-yl)-2,8- diazaspiro[4.5]decan-1-one | 453.07 |
| 50 | 22 and 41, method for Ex. 25 | 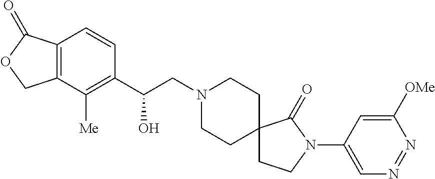 [SMILES: COc1cc(N2CCC3(CCN(C[C@H](O)c4ccc5c(c4C)COC5=O)CC3)C2=O)cnn1] (R)-8-(2-hydroxy-2-(4-methyl-1-oxo-1,3-dihydroisobenzofuran-5- yl)ethyl)-2-(6-methoxypyridazin-4-yl)-2,8-diazaspiro[4.5]decan- 1-one | 453.02 |
| 51 | 22 and 36, method for Ex. 24 | 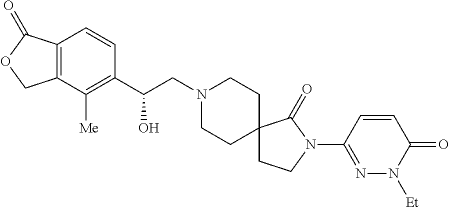 [SMILES: CCn1nc(N2CCC3(CCN(C[C@H](O)c4ccc5c(c4C)COC5=O)CC3)C2=O)ccc1=O] (R)-2-(1-ethyl-6-oxo-1,6-dihydropyridazin-3-yl)-8-(2-hydroxy-2- (4-methyl-1-oxo-1,3-dihydroisobenzofuran-5-yl)ethyl)-2,8- diazaspiro[4.5]decan-1-one | 467.11 |
| 52 | 22 and 42 method for Ex. 25 | 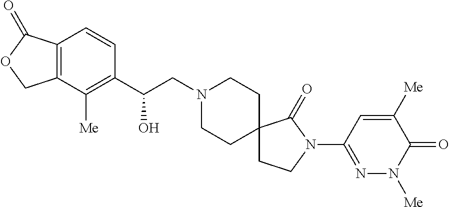 [SMILES: Cc1c([C@@H](O)CN2CCC3(CC2)CCN(c2cc(C)c(=O)n(C)n2)C3=O)ccc2c1COC2=O] (R)-2-(1,5-dimethyl-6-oxo-1,6-dihydropyridazin-3-yl)-8-(2- hydroxy-2-(4-methyl-1-oxo-1,3-dihydroisobenzofuran-5- yl)ethyl)-2,8-diazaspiro[4.5]decan-1-one | 467.03 |
| 53 | 22 and 37, method for Ex. 25 | 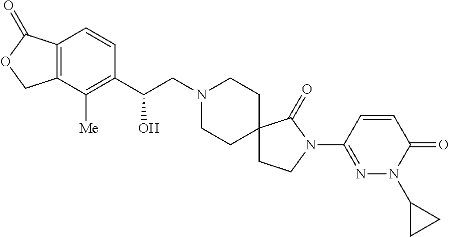 [SMILES: Cc1c([C@@H](O)CN2CCC3(CC2)CCN(c2ccc(=O)n(C4CC4)n2)C3=O)ccc2c1COC2=O] (R)-2-(1-cyclopropyl-6-oxo-1,6-dihydropyridazin-3-yl)-8-(2- hydroxy-2-(4-methyl-1-oxo-1,3-dihydroisobenzofuran-5- yl)ethyl)-2,8-diazaspiro[4.5]decan-1-one | 479.10 |
| 54 | 22 and 38, method for Ex. 24 | 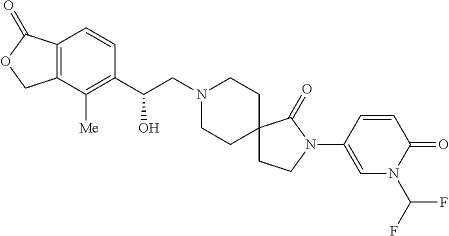 [SMILES: Cc1c([C@@H](O)CN2CCC3(CC2)CCN(c2ccc(=O)n(C(F)F)c2)C3=O)ccc2c1COC2=O] (R)-2-(1-(difluoromethyl)-6-oxo-1,6-dihydropyridin-3-yl)-8-(2- hydroxy-2-(4-methyl-1-oxo-1,3-dihydroisobenzofuran-5- yl)ethyl)-2,8-diazaspiro[4.5]decan-1-one | 488.02 |
| 55 | 22 and 39, method for Ex. 25 | 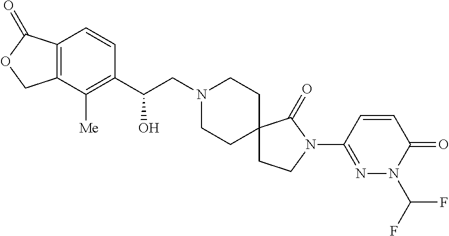 [SMILES: Cc1c([C@@H](O)CN2CCC3(CC2)CCN(c2ccc(=O)n(C(F)F)n2)C3=O)ccc2c1COC2=O] (R)-2-(1-(difluoromethyl)-6-oxo-1,6-dihydropyridazin-3-yl)-8-(2- hydroxy-2-(4-methyl-1-oxo-1,3-dihydroisobenzofuran-5- yl)ethyl)-2,8-diazaspiro[4.5]decan-1-one | 489.09 |

### Example 56

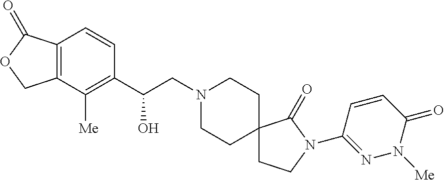 [SMILES: Cc1c([C@@H](O)CN2CCC3(CC2)CCN(c2ccc(=O)n(C)n2)C3=O)ccc2c1COC2=O]

To 2-(1-methyl-6-oxo-1,6-dihydropyridazin-3-yl)-2,8-diazaspiro[4.5]decan-1-one (INTERMEDIATE 34, 100 mg, 0.381 mmol) in ethanol (2 mL) was added (R)-4-methyl-5-(oxiran-2-yl)isobenzofuran-1(3H)-one (INTERMEDIATE 4A, 87 mg, 0.457 mmol). The reaction mixture was heated at 90° C. overnight. The reaction mixture was evaporated, and the crude product was purified by prep-TLC (2000 μm, 8% MeOH/EtOAc as eluent) to provide (R)-8-(2-hydroxy-2-(4-methyl-1-oxo-1,3-dihydroisobenzofuran-5-yl)ethyl)-2-(1-methyl-6-oxo-1,6-dihydropyridazin-3-yl)-2,8-diazaspiro[4.5]decan-1-one. ¹H NMR (500 MHz, CDCl₃), δ 8.51 (d, J=10.0 Hz, 1H), 7.81 (ABq, J=14.4 Hz, Δδ=8.0 Hz, 2H), 6.97 (d, J=10.0 Hz, 1H), 5.26 (s, 2H), 5.12 (dd, J=10.6, 3.1 Hz, 1H), 3.86 (t, J=7.0 Hz, 2H), 3.74 (s, 3H), 3.21-3.18 (m, 1H), 2.90-2.86 (m, 1H), 2.62-2.55 (m, 2H), 2.46-2.41 (m, 1H), 2.34-2.31 (m, 1H), 2.30 (s, 3H), 2.16-2.06 (m, 4H), 1.66-1.60 (m, 2H). LC-MS (IE, m/z): 453.08 [M+1]⁺.

The compounds in Table 9 were prepared in an analogous fashion to EXAMPLE 57 starting from epoxides and amines specified below.

TABLE 9

| | | | LC/MS |
|---|---|---|---|
| EX. | INT. | EXAMPLE STRUCTURE/NAME | [M + 1]⁺ |
| 57 | 4B and 34 | 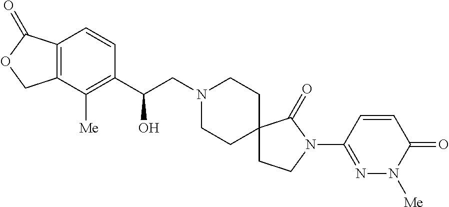 [SMILES: Cc1c([C@H](O)CN2CCC3(CC2)CCN(c2ccc(=O)n(C)n2)C3=O)ccc2c1COC2=O] (S)-8-(2-hydroxy-2-(4-methyl-1-oxo-1,3- dihydroisobenzofuran-5-yl)ethyl)-2-(1-methyl-6-oxo-1,6- dihydropyridazin-3-yl)-2,8-diazaspiro[4.5]decan-1-one [faster eluting enantiomer from SFC separation using CHIRALCEL ® AS column] | 452.96 |
| 58 | 4C and 34 | 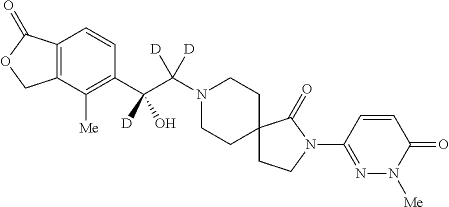 [SMILES: [2H]C([2H])(N1CCC2(CCN(c3ccc(=O)n(C)n3)C2=O)CC1)[C@]([2H])(O)c1ccc2c(c1C)COC2=O] (R)-8-(2-hydroxy-2-(4-methyl-1-oxo-1,3- dihydroisobenzofuran-5-yl)ethyl-d₃)-2-(1-methyl-6-oxo-1,6- dihydropyridazin-3-yl)-2,8-diazaspiro[4.5]decan-1-one, faster eluting enantiomer from SFC separation using CHIRALCEL ® AS column | 456.14 |
| 59 | 4C and 34 | 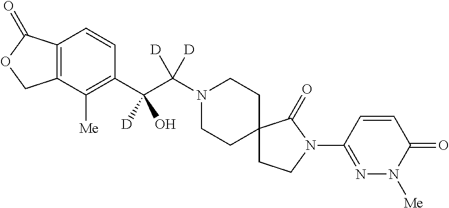 [SMILES: [2H]C([2H])(N1CCC2(CCN(c3ccc(=O)n(C)n3)C2=O)CC1)[C@@]([2H])(O)c1ccc2c(c1C)COC2=O] (S)-8-(2-hydroxy-2-(4-methyl-1-oxo-1,3- dihydroisobenzofuran-5-yl)ethyl-d₃)-2-(1-methyl-6-oxo-1,6- dihydropyridazin-3-yl)-2,8-diazaspiro[4.5]decan-1-one, slower eluting enantiomer from SFC separation using CHIRALCEL ® AS column | 456.18 |
| 60 | 4A and 45 | 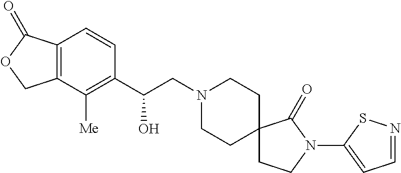 [SMILES: Cc1c([C@@H](O)CN2CCC3(CC2)CCN(c2ccns2)C3=O)ccc2c1COC2=O] (R)-8-(2-hydroxy-2-(4-methyl-1-oxo-1,3- dihydroisobenzofuran-5-yl)ethyl)-2-(isothiazol-5-yl)-2,8- diazaspiro[4.5]decan-1-one | 428.05 |
| 61 | 4A and 46 | 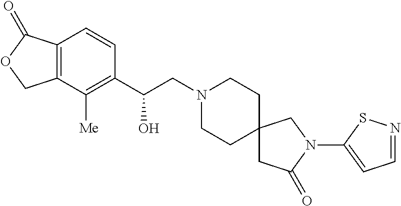 [SMILES: Cc1c([C@@H](O)CN2CCC3(CC2)CC(=O)N(c2ccns2)C3)ccc2c1COC2=O] (R)-8-(2-hydroxy-2-(4-methyl-1-oxo-1,3- dihydroisobenzofuran-5-yl)ethyl)-2-(isothiazol-5-yl)-2,8- diazaspiro[4.5]decan-3-one | 428.15 |
| 62 | 4A and 47 | 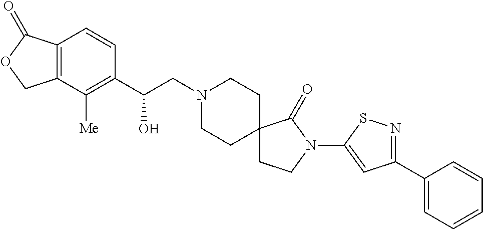 [SMILES: Cc1c([C@@H](O)CN2CCC3(CC2)CCN(c2cc(-c4ccccc4)ns2)C3=O)ccc2c1COC2=O] (R)-8-(2-hydroxy-2-(4-methyl-1-oxo-1,3- dihydroisobenzofuran-5-yl)ethyl)-2-(3-phenylisothiazol-5- yl)-2,8-diazaspiro[4.5]decan-1-one | 504.11 |
| 63 | 4A and 48 | 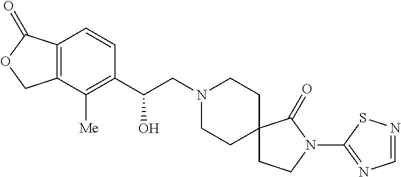 [SMILES: Cc1c([C@@H](O)CN2CCC3(CC2)CCN(c2ncns2)C3=O)ccc2c1COC2=O] (R)-8-(2-hydroxy-2-(4-methyl-1-oxo-1,3- dihydroisobenzofuran-5-yl)ethyl)-2-(1,2,4-thiadiazol-5-yl)- 2,8-diazaspiro[4.5]decan-1-one | 428.90 |
| 64 | 4A and 49 | 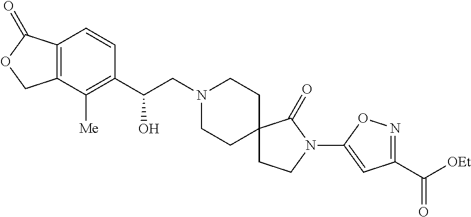 [SMILES: CCOC(=O)c1cc(N2CCC3(CCN(C[C@H](O)c4ccc5c(c4C)COC5=O)CC3)C2=O)on1] (R)-ethyl 5-(8-(2-hydroxy-2-(4-methyl-1-oxo-1,3- dihydroisobenzofuran-5-yl)ethyl)-1-oxo-2,8- diazaspiro[4.5]decan-2-yl)isoxazole-3-carboxylate | 483.94 |
| 65 | 4A and 44 | 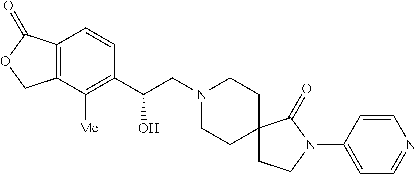 [SMILES: Cc1c([C@@H](O)CN2CCC3(CC2)CCN(c2ccncc2)C3=O)ccc2c1COC2=O] (R)-8-(2-hydroxy-2-(4-methyl-1-oxo-1,3- dihydroisobenzofuran-5-yl)ethyl)-2-(pyridin-4-yl)-2,8- diazaspiro[4.5]decan-1-one | 422.02 |
| 66 | 4A and 50 | 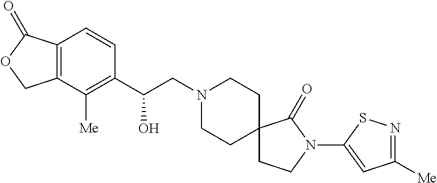 [SMILES: Cc1cc(N2CCC3(CCN(C[C@H](O)c4ccc5c(c4C)COC5=O)CC3)C2=O)sn1] (R)-8-(2-hydroxy-2-(4-methyl-1-oxo-1,3- dihydroisobenzofuran-5-yl)ethyl)-2-(3-methylisothiazol-5- yl)-2,8-diazaspiro[4.5]decan-1-one | 441.91 |
| 67 | 4A and 51 | 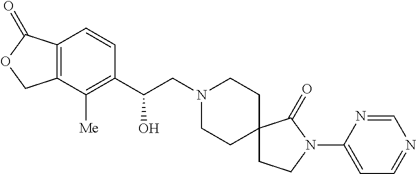 [SMILES: Cc1c([C@@H](O)CN2CCC3(CC2)CCN(c2ccncn2)C3=O)ccc2c1COC2=O] (R)-8-(2-hydroxy-2-(4-methyl-1-oxo-1,3- dihydroisobenzofuran-5-yl)ethyl)-2-(pyrimidin-4-yl)-2,8- diazaspiro[4.5]decan-1-one | 422.97 |
| 68 | 6A and 45 | 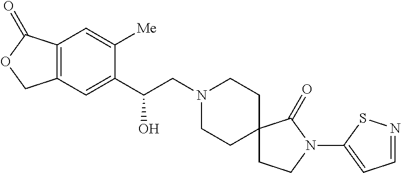 [SMILES: Cc1cc2c(cc1[C@@H](O)CN1CCC3(CC1)CCN(c1ccns1)C3=O)COC2=O] (R)-8-(2-hydroxy-2-(6-methyl-1-oxo-1,3- dihydroisobenzofuran-5-yl)ethyl)-2-(isothiazol-5-yl)-2,8- diazaspiro[4.5]decan-1-one | 427.94 |
| 69 | 6B and 45 | 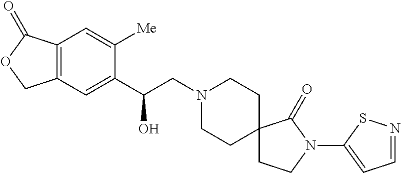 [SMILES: Cc1cc2c(cc1[C@H](O)CN1CCC3(CC1)CCN(c1ccns1)C3=O)COC2=O] (S)-8-(2-hydroxy-2-(6-methyl-1-oxo-1,3- dihydroisobenzofuran-5-yl)ethyl)-2-(isothiazol-5-yl)-2,8- diazaspiro[4.5]decan-1-one | 427.92 |
| 70 | 14B and 45 | 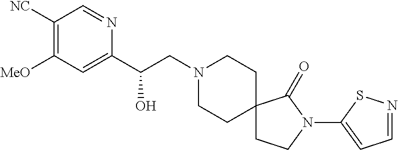 [SMILES: COc1cc([C@@H](O)CN2CCC3(CC2)CCN(c2ccns2)C3=O)ncc1C#N] (S)-6-(1-hydroxy-2-(2-(isothiazol-5-yl)-1-oxo-2,8- diazaspiro[4.5]decan-8-yl)ethyl)-4-methoxynicotinonitrile | 413.92 |
| 71 | 4A and 52 | 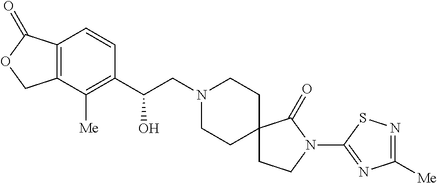 [SMILES: Cc1nsc(N2CCC3(CCN(C[C@H](O)c4ccc5c(c4C)COC5=O)CC3)C2=O)n1] (R)-8-(2-hydroxy-2-(4-methyl-1-oxo-1,3- dihydroisobenzofuran-5-yl)ethyl)-2-(3-methyl-1,2,4- thiadiazol-5-yl)-2,8-diazaspiro[4.5]decan-1-one | 442.95 |
| 72 | 4A and 53 | 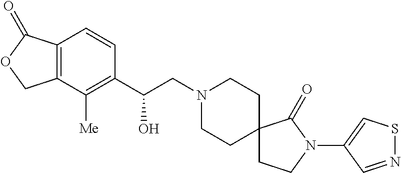 [SMILES: Cc1c([C@@H](O)CN2CCC3(CC2)CCN(c2cnsc2)C3=O)ccc2c1COC2=O] (R)-8-(2-hydroxy-2-(4-methyl-1-oxo-1,3- dihydroisobenzofuran-5-yl)ethyl)-2-(isothiazol-4-yl)-2,8- diazaspiro[4.5]decan-1-one | 427.95 |
| 73 | 4A and 54 | 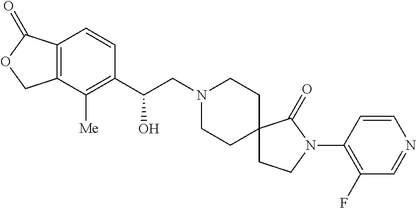 [SMILES: Cc1c([C@@H](O)CN2CCC3(CC2)CCN(c2ccncc2F)C3=O)ccc2c1COC2=O] (R)-2-(3-fluoropyridin-4-yl)-8-(2-hydroxy-2-(4-methyl-1- oxo-1,3-dihydroisobenzofuran-5-yl)ethyl)-2,8- diazaspiro[4.5]decan-1-one | 440.02 |
| 74 | 4A and 55 | 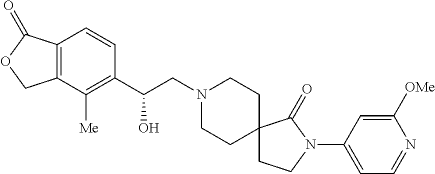 [SMILES: COc1cc(N2CCC3(CCN(C[C@H](O)c4ccc5c(c4C)COC5=O)CC3)C2=O)ccn1] (R)-8-(2-hydroxy-2-(4-methyl-1-oxo-1,3- dihydroisobenzofuran-5-yl)ethyl)-2-(2-methoxypyridin-4- yl)-2,8-diazaspiro[4.5]decan-1-one | 452.05 |
| 75 | 4A and 56 | 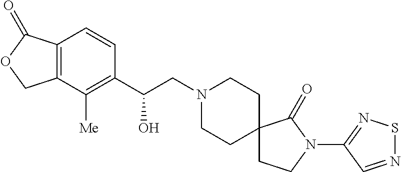 [SMILES: Cc1c([C@@H](O)CN2CCC3(CC2)CCN(c2cnsn2)C3=O)ccc2c1COC2=O] (R)-8-(2-hydroxy-2-(4-methyl-1-oxo-1,3- dihydroisobenzofuran-5-yl)ethyl)-2-(1,2,5-thiadiazol-3-yl)- 2,8-diazaspiro[4.5]decan-1-one | 428.98 |
| 76 | 4A and 57 | 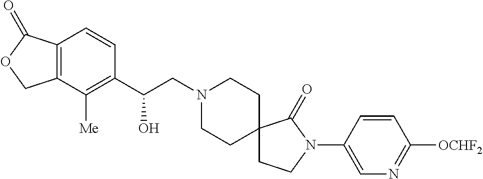 [SMILES: Cc1c([C@@H](O)CN2CCC3(CC2)CCN(c2ccc(OC(F)F)nc2)C3=O)ccc2c1COC2=O] (R)-2-(6-(difluoromethoxy)pyridin-3-yl)-8-(2-hydroxy-2-(4- methyl-1-oxo-1,3-dihydroisobenzofuran-5-yl)ethyl)-2,8- diazaspiro[4.5]decan-1-one | 488.00 |
| 77 | 4A and 58 | 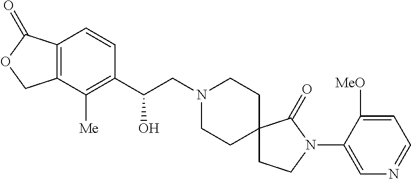 [SMILES: COc1ccncc1N1CCC2(CCN(C[C@H](O)c3ccc4c(c3C)COC4=O)CC2)C1=O] (R)-8-(2-hydroxy-2-(4-methyl-1-oxo-1,3- dihydroisobenzofuran-5-yl)ethyl)-2-(4-methoxypyridin-3- yl)-2,8-diazaspiro[4.5]decan-1-one | 452.00 |
| 78 | 4A and 29 | 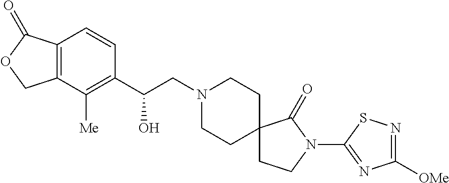 [SMILES: COc1nsc(N2CCC3(CCN(C[C@H](O)c4ccc5c(c4C)COC5=O)CC3)C2=O)n1] (R)-8-(2-hydroxy-2-(4-methyl-1-oxo-1,3- dihydroisobenzofuran-5-yl)ethyl)-2-(3-methoxy-1,2,4- thiadiazol-5-yl)-2,8-diazaspiro[4.5]decan-1-one | 458.99 |
| 79 | 4A and 59 | 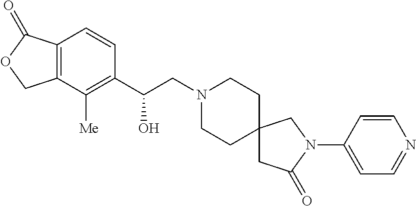 [SMILES: Cc1c([C@@H](O)CN2CCC3(CC2)CC(=O)N(c2ccncc2)C3)ccc2c1COC2=O] (R)-8-(2-hydroxy-2-(4-methyl-1-oxo-1,3- dihydroisobenzofuran-5-yl)ethyl)-2-(pyridin-4-yl)-2,8- diazaspiro[4.5]decan-3-one | 422.06 |
| 80 | 4A and 60 | 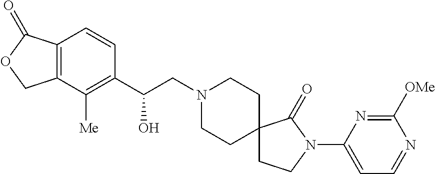 [SMILES: COc1nccc(N2CCC3(CCN(C[C@H](O)c4ccc5c(c4C)COC5=O)CC3)C2=O)n1] (R)-8-(2-hydroxy-2-(4-methyl-1-oxo-1,3- dihydroisobenzofuran-5-yl)ethyl)-2-(2-methoxypyrimidin-4- yl)-2,8-diazaspiro[4.5]decan-1-one | 453.07 |
| 81 | 4A and 61 | 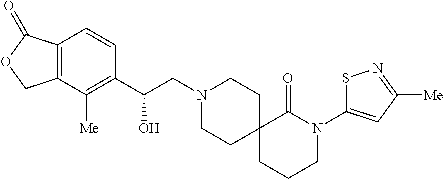 [SMILES: Cc1cc(N2CCCC3(CCN(C[C@H](O)c4ccc5c(c4C)COC5=O)CC3)C2=O)sn1] (R)-9-(2-hydroxy-2-(4-methyl-1-oxo-1,3- dihydroisobenzofuran-5-yl)ethyl)-2-(3-methylisothiazol-5- yl)-2,9-diazaspiro[5.5]undecan-1-one | 456.03 |
| 82 | 4A and 62 | 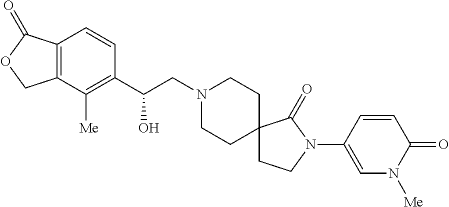 [SMILES: Cc1c([C@@H](O)CN2CCC3(CC2)CCN(c2ccc(=O)n(C)c2)C3=O)ccc2c1COC2=O] (R)-8-(2-hydroxy-2-(4-methyl-1-oxo-1,3- dihydroisobenzofuran-5-yl)ethyl)-2-(1-methyl-6-oxo-1,6- dihydropyridin-3-yl)-2,8-diazaspiro[4.5]decan-1-one | 452.07 |
| 83 | 4A and 63 | 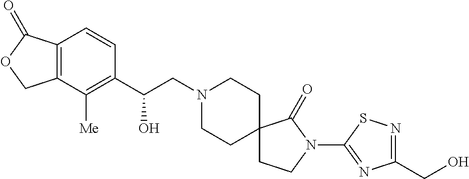 [SMILES: Cc1c([C@@H](O)CN2CCC3(CC2)CCN(c2nc(CO)ns2)C3=O)ccc2c1COC2=O] (R)-8-(2-hydroxy-2-(4-methyl-1-oxo-1,3- dihydroisobenzofuran-5-yl)ethyl)-2-(3-(hydroxymethyl)- 1,2,4-thiadiazol-5-yl)-2,8-diazaspiro[4.5]decan-1-one | 459.05 |
| 84 | 4A and 64 | 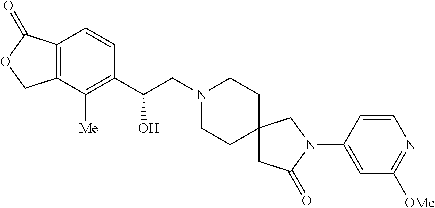 [SMILES: COc1cc(N2CC3(CCN(C[C@H](O)c4ccc5c(c4C)COC5=O)CC3)CC2=O)ccn1] (R)-8-(2-hydroxy-2-(4-methyl-1-oxo-1,3- dihydroisobenzofuran-5-yl)ethyl)-2-(2-methoxypyridin-4- yl)-2,8-diazaspiro[4.5]decan-3-one | 452.04 |
| 85 | 4A and 65 | 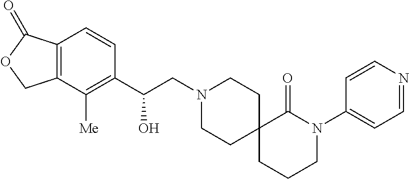 [SMILES: Cc1c([C@@H](O)CN2CCC3(CCCN(c4ccncc4)C3=O)CC2)ccc2c1COC2=O] (R)-9-(2-hydroxy-2-(4-methyl-1-oxo-1,3- dihydroisobenzofuran-5-yl)ethyl)-2-(pyridin-4-yl)-2,9- diazaspiro[5.5]undecan-1-one | 436.13 |
| 86 | 4A and 66 | 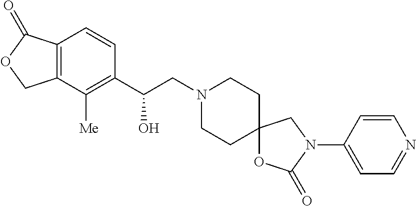 [SMILES: Cc1c([C@@H](O)CN2CCC3(CC2)CN(c2ccncc2)C(=O)O3)ccc2c1COC2=O] (R)-8-(2-hydroxy-2-(4-methyl-1-oxo-1,3- dihydroisobenzofuran-5-yl)ethyl)-3-(pyridin-4-yl)-1-oxa-3,8- diazaspiro[4.5]decan-2-one | 424.09 |
| 87 | 4A and 67 | 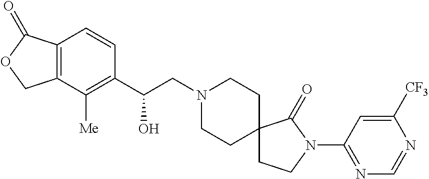 [SMILES: Cc1c([C@@H](O)CN2CCC3(CC2)CCN(c2cc(C(F)(F)F)ncn2)C3=O)ccc2c1COC2=O] (R)-8-(2-hydroxy-2-(4-methyl-1-oxo-1,3- dihydroisobenzofuran-5-yl)ethyl)-2-(6- (trifluoromethyl)pyrimidin-4-yl)-2,8-diazaspiro[4.5]decan-1- one | 491.4 |
| 88 | 4A and 68 | 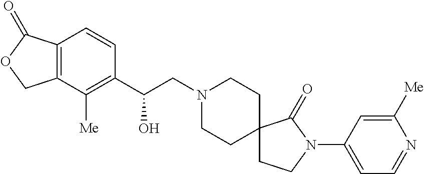 [SMILES: Cc1cc(N2CCC3(CCN(C[C@H](O)c4ccc5c(c4C)COC5=O)CC3)C2=O)ccn1] (R)-8-(2-hydroxy-2-(4-methyl-1-oxo-1,3- dihydroisobenzofuran-5-yl)ethyl)-2-(2-methylpyridin-4-yl)- 2,8-diazaspiro[4.5]decan-1-one | 436.05 |
| 89 | 9A and 60 | 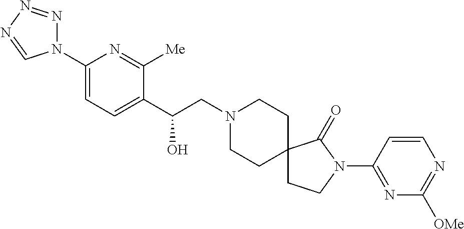 [SMILES: COc1nccc(N2CCC3(CCN(C[C@H](O)c4ccc(-n5cnnn5)nc4C)CC3)C2=O)n1] (R)-8-(2-hydroxy-2-(2-methyl-6-(1H-tetrazol-1-yl)pyridin-3- yl)ethyl)-2-(2-methoxypyrimidin-4-yl)-2,8- diazaspiro[4.5]decan-1-one | 466.07 |
| 90 | 4A and 69 | 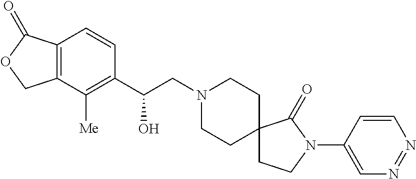 (R)-8-(2-hydroxy-2-(4-methyl-1-oxo-1,3- dihydroisobenzofuran-5-yl)ethyl)-2-(pyridazin-4-yl)-2,8- diazaspiro[4.5]decan-1-one | 423.02 |
| 91 | 4A and 70 | 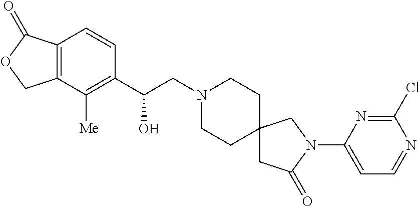 (R)-2-(2-chloropyrimidin-4-yl)-8-(2-hydroxy-2-(4-methyl-1- oxo-1,3-dihydroisobenzofuran-5-yl)ethyl)-2,8- diazaspiro[4.5]decan-3-one | 457.24 |
| 92 | 4A and 71 | 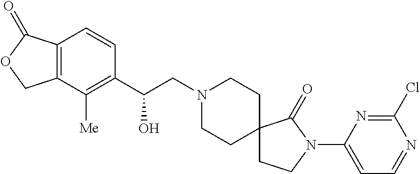 (R)-2-(2-chloropyrimidin-4-yl)-8-(2-hydroxy-2-(4-methyl-1- oxo-1,3-dihydroisobenzofuran-5-yl)ethyl)-2,8- diazaspiro[4.5]decan-1-one | 457.03 |
| 93 | 9A and 34 | 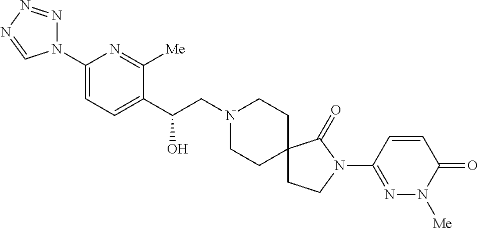 (R)-8-(2-hydroxy-2-(2-methyl-6-(1H-tetrazol-1-yl)pyridin-3- yl)ethyl)-2-(1-methyl-6-oxo-1,6-dihydropyridazin-3-yl)-2,8- diazaspiro[4.5]decan-1-one | 466.08 |
| 94 | 4A and 72 | 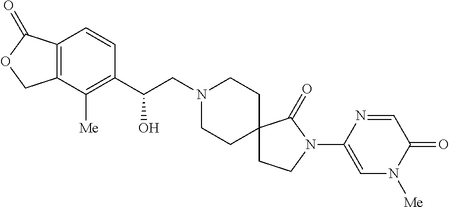 [SMILES: Cc1c([C@@H](O)CN2CCC3(CC2)CCN(c2cn(C)c(=O)cn2)C3=O)ccc2c1COC2=O] (R)-8-(2-hydroxy-2-(4-methyl-1-oxo-1,3- dihydroisobenzofuran-5-yl)ethyl)-2-(4-methyl-5-oxo-4,5- dihydropyrazin-2-yl)-2,8-diazaspiro[4.5]decan-1-one | 453.00 |
| 95 | 4A and 73 | 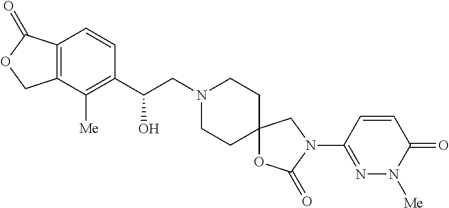 [SMILES: Cc1c([C@@H](O)CN2CCC3(CC2)CN(c2ccc(=O)n(C)n2)C(=O)O3)ccc2c1COC2=O] (R)-8-(2-hydroxy-2-(4-methyl-1-oxo-1,3- dihydroisobenzofuran-5-yl)ethyl)-3-(1-methyl-6-oxo-1,6- dihydropyridazin-3-yl)-1-oxa-3,8-diazaspiro[4.5]decan-2- one | 455.02 |
| 96 | 11B and 34 | 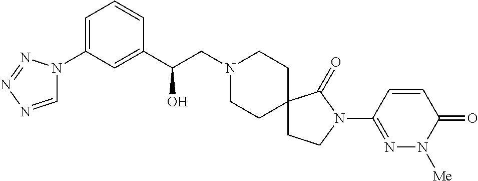 [SMILES: Cn1nc(N2CCC3(CCN(C[C@@H](O)c4cccc(-n5cnnn5)c4)CC3)C2=O)ccc1=O] (S)-8-(2-(3-(1H-tetrazol-1-yl)phenyl)-2-hydroxyethyl)-2-(1- methyl-6-oxo-1,6-dihydropyridazin-3-yl)-2,8- diazaspiro[4.5]decan-1-one | 451.01 |
| 97 | 4A and 74 | 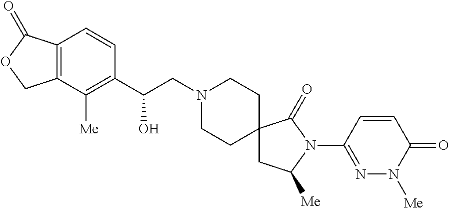 [SMILES: Cc1c([C@@H](O)CN2CCC3(CC2)C[C@H](C)N(c2ccc(=O)n(C)n2)C3=O)ccc2c1COC2=O] (S)-8-((R)-2-hydroxy-2-(4-methyl-1-oxo-1,3- dihydroisobenzofuran-5-yl)ethyl)-3-methyl-2-(1-methyl-6- oxo-1,6-dihydropyridazin-3-yl)-2,8-diazaspiro[4.5]decan-1- one | 467.28 |
| 98 | 4A and 75 | 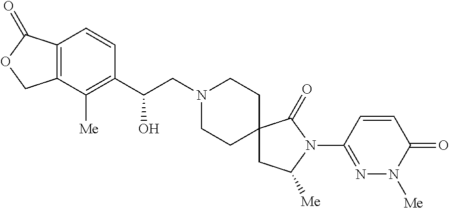 [SMILES: Cc1c([C@@H](O)CN2CCC3(CC2)C[C@@H](C)N(c2ccc(=O)n(C)n2)C3=O)ccc2c1COC2=O] (R)-8-((R)-2-hydroxy-2-(4-methyl-1-oxo-1,3- dihydroisobenzofuran-5-yl)ethyl)-3-methyl-2-(1-methyl-6- oxo-1,6-dihydropyridazin-3-yl)-2,8-diazaspiro[4.5]decan-1- one | 467.27 |
| 99 | 9A and 48 | 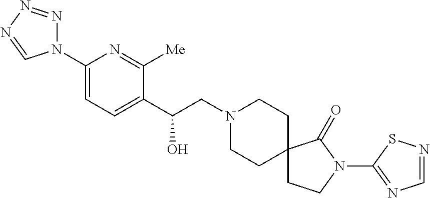 [SMILES: Cc1nc(-n2cnnn2)ccc1[C@@H](O)CN1CCC2(CC1)CCN(c1ncns1)C2=O] (R)-8-(2-hydroxy-2-(2-methyl-6-(1H-tetrazol-1-yl)pyridin-3- yl)ethyl)-2-(1,2,4-thiadiazol-5-yl)-2,8-diazaspiro[4.5]decan- 1-one | 442.1 |
| 100 | 7A and 45 | 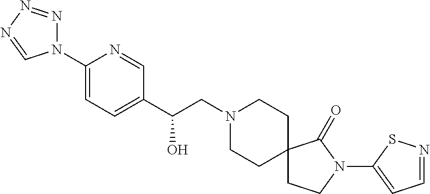 [SMILES: O=C1N(c2ccns2)CCC12CCN(C[C@H](O)c1ccc(-n3cnnn3)nc1)CC2] (R)-8-(2-(6-(1H-tetrazol-1-yl)pyridin-3-yl)-2-hydroxyethyl)- 2-(isothiazol-5-yl)-2,8-diazaspiro[4.5]decan-1-one | 427.2 |
| 101 | 7B and 45 | 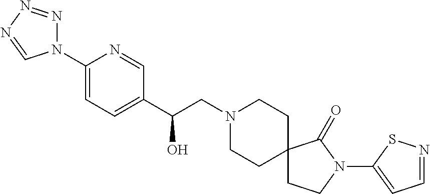 [SMILES: O=C1N(c2ccns2)CCC12CCN(C[C@@H](O)c1ccc(-n3cnnn3)nc1)CC2] (S)-8-(2-(6-(1H-tetrazol-1-yl)pyridin-3-yl)-2-hydroxyethyl)- 2-(isothiazol-5-yl)-2,8-diazaspiro[4.5]decan-1-one | 427.2 |
| 102 | 4A and 76 | 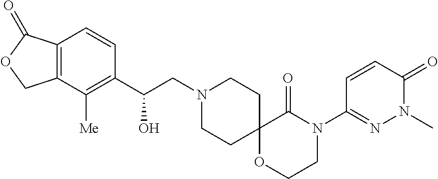 [SMILES: Cc1c([C@@H](O)CN2CCC3(CC2)OCCN(c2ccc(=O)n(C)n2)C3=O)ccc2c1COC2=O] (R)-9-(2-hydroxy-2-(4-methyl-1-oxo-1,3- dihydroisobenzofuran-5-yl)ethyl)-4-(1-methyl-6-oxo-1,6- dihydropyridazin-3-yl)-1-oxa-4,9-diazaspiro[5.5]undecan-5- one | 469 |
| 103 | 4A and 77 | 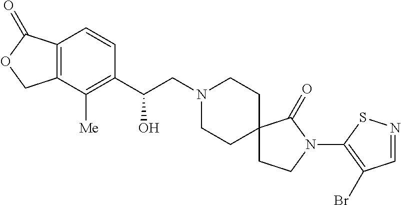 [SMILES: Cc1c([C@@H](O)CN2CCC3(CC2)CCN(c2sncc2Br)C3=O)ccc2c1COC2=O] (R)-2-(4-bromoisothiazol-5-yl)-8-(2-hydroxy-2-(4-methyl-1- oxo-1,3-dihydroisobenzofuran-5-yl)ethyl)-2,8- diazaspiro[4.5]decan-1-one | 507.89 |
| 104 | 4A and 78 | 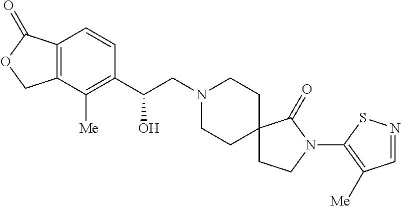 [SMILES: Cc1cnsc1N1CCC2(CCN(C[C@H](O)c3ccc4c(c3C)COC4=O)CC2)C1=O] (R)-8-(2-hydroxy-2-(4-methyl-1-oxo-1,3- dihydroisobenzofuran-5-yl)ethyl)-2-(4-methylisothiazol-5- yl)-2,8-diazaspiro[4.5]decan-1-one | 441.94 |
| 105 | 4A and 79 | 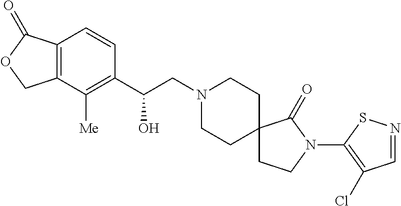 [SMILES: Cc1c([C@@H](O)CN2CCC3(CC2)CCN(c2sncc2Cl)C3=O)ccc2c1COC2=O] (R)-2-(4-chloroisothiazol-5-yl)-8-(2-hydroxy-2-(4-methyl-1- oxo-1,3-dihydroisobenzofuran-5-yl)ethyl)-2,8- diazaspiro[4.5]decan-1-one | 461.85 |

### Example 106

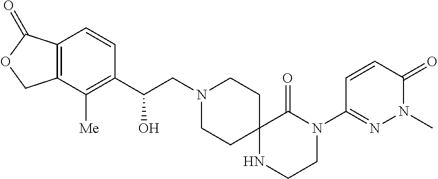 [SMILES: Cc1c([C@@H](O)CN2CCC3(CC2)NCCN(c2ccc(=O)n(C)n2)C3=O)ccc2c1COC2=O]

To (9H-fluoren-9-yl)methyl 4-(1-methyl-6-oxo-1,6-dihydropyridazin-3-yl)-5-oxo-1,4,9-triazaspiro[5.5]undecane-1-carboxylate (INTERMEDIATE 43, 29.5 mg, 0.055 mmol) in ethanol (2 mL) was added (R)-4-methyl-5-(oxiran-2-yl)isobenzofuran-1(3H)-one (INTERMEDIATE 4A, 10.5 mg, 0.055 mmol) and triethylamine (0.023 mL, 0.165 mmol). The reaction mixture was heated at 80° C. overnight. The reaction mixture was evaporated, and the crude product was purified by prep-TLC (2000 μm, 10% MeOH/DCM as eluent) to remove unreacted starting materials. The partially purified product was further purified by SFC-HPLC using a CHIRALCEL® OD column to provide (R)-9-(2-hydroxy-2-(4-methyl-1-oxo-1,3-dihydroisobenzofuran-5-yl)ethyl)-4-(1-methyl-6-oxo-1,6-dihydropyridazin-3-yl)-1,4,9-triazaspiro[5.5]undecan-5-one. LC-MS (IE, m/z): 468.06 (M+1)⁺.

### Example 107, 108, 109

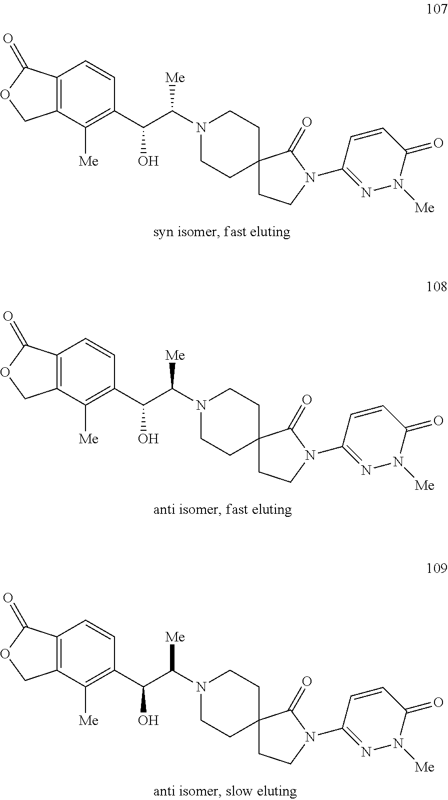 [SMILES: Cc1c([C@@H](O)[C@H](C)N2CCC3(CCN(c4ccc(=O)n(C)n4)C3=O)CC2)ccc2c1COC2=O ; Cc1c([C@@H](O)[C@@H](C)N2CCC3(CCN(c4ccc(=O)n(C)n4)C3=O)CC2)ccc2c1COC2=O ; Cc1c([C@H](O)[C@@H](C)N2CCC3(CCN(c4ccc(=O)n(C)n4)C3=O)CC2)ccc2c1COC2=O]

### Syn isomer, fast eluting 107: 8-((1R,2S)-1-hydroxy-1-(4-methyl-1-oxo-1,3-dihydroisobenzofuran-5-yl)propan-2-yl)-2-(1-methyl-6-oxo-1,6-dihydropyridazin-3-yl)-2,8-diazaspiro[4.5]decan-1-one

### Anti isomer, fast eluting 108: 8-((1R,2R)-1-hydroxy-1-(4-methyl-1-oxo-1,3-dihydroisobenzofuran-5-yl)propan-2-yl)-2-(1-methyl-6-oxo-1,6-dihydropyridazin-3-yl)-2,8-diazaspiro[4.5]decan-1-one

### Anti isomer, slow eluting 109: 8-41S,2S)-1-hydroxy-1-(4-methyl-1-oxo-1,3-dihydroisobenzofuran-5-yl)propan-2-yl)-2-(1-methyl-6-oxo-1,6-dihydropyridazin-3-yl)-2,8-diazaspiro[4.5]decan-1-one

To a MW vial was charged with 5-(1,2-dihydroxypropyl)-4-methylisobenzofuran-1(3H)-one (INTERMEDIATE 5, 150 mg, 0.675 mmol), 2-(1-methyl-6-oxo-1,6-dihydropyridazin-3-yl)-2,8-diazaspiro[4.5]decan-1-one (INTERMEDIATE 34, 212 mg, 0.810 mmol), 2-(dicyclohexylphosphino)-1-phenyl-1H-pyrrole (41.2 mg, 0.121 mmol), ruthenium carbonyl (25.9 mg, 0.040 mmol) and tert-amyl alcohol (1.4 mL). The MW vial was sealed, degassed, and heated at 145° C. for 60 h. The reaction mixture was diluted with DCM, dry-loaded to silica gel column, purified by silica gel column (0-10% MeOH/DCM as eluent) to give the syn and anti products, which were then respectively resolved by SFC-HPLC using a CHIRALPAK® AS-H column to provide the title compounds. LC-MS (IE, m/z): 466.88 (M+1)⁺.

### Example 110

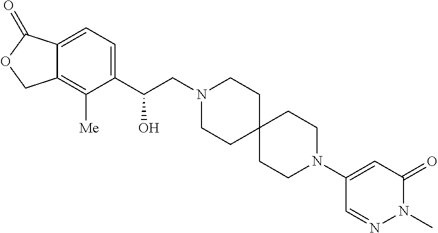 [SMILES: Cc1c([C@@H](O)CN2CCC3(CC2)CCN(c2cnn(C)c(=O)c2)CC3)ccc2c1COC2=O]

### (R)-5-(9-(2-hydroxy-2-(4-methyl-1-oxo-1,3-dihydroisobenzofuran-5-yl)ethyl)-3,9-diazaspiro[5.5]undecan-3-yl)-2-methylpyridazin-3(2H)-one

Example 110 was prepared in an analog fashion to Example 56 using Intermediates 4A and 83. LC/MS [(M+1)]′=453.

Preparative TLC separation (5% MeOH in CH₂Cl₂) provided the free base, which was dissolved in DCM and treated with 1N HCl in ether (1.05 eq.). After 1 h, the solvent was evaporated and the product dried under high vacuum to provide the title compound. LC/MS: [(M+1)]⁺=449

### Example 111

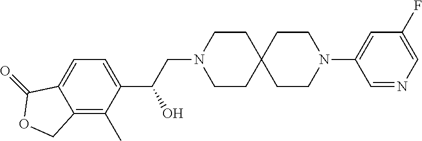 [SMILES: Cc1c([C@@H](O)CN2CCC3(CC2)CCN(c2cncc(F)c2)CC3)ccc2c1COC2=O]

### 9-(5-fluoropyridin-3-yl)-3-[(2R)-2-hydroxy-2-(4-methyl-1-oxo-1,3-dihydro-2-benzofuran-5-yl)ethyl]-9-aza-3-azoniaspiro[5.5]undecane

To a solution of (R)-5-(1-hydroxy-2-(3,9-diazaspiro[5.5]undecan-3-yl)ethyl)-4-methylisobenzofuran-1(3H)-one (INTERMEDIATE 81, 35 mg, 0.102 mmol) in anhydrous DMSO (1 mL) was quickly added Cu(OAc)₂ (10 mg, 0.055 mmol), (5-fluoropyridin-3-yl)boronic acid (29 mg, 0.204 mmol) and then DBU (50 μL, 0.31 mmol). The reaction was sealed and heated at 100° C. in microwave for 30 min. LC-MS showed that the product was formed. The reaction was cooled to ambient temperature and partitioned between EtOAc (4 mL×2) and ammonium (2N, 2 mL). The organic phase was combined and evaporated in vacuum. The residue was dissolved in DMSO (1.5 mL). The crude product was purified by using reversed-phase HPLC (acetonitrile with 0.1% formic acid: water with 0.1% formic acid from 10% to 60%) to give the product as a yellow solid. LC-MS (IE, m/z): 440 [M+1]⁺.

### Example 112

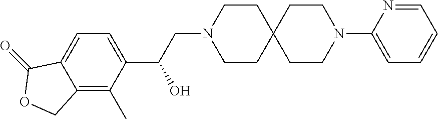 [SMILES: Cc1c([C@@H](O)CN2CCC3(CC2)CCN(c2ccccn2)CC3)ccc2c1COC2=O]

### 3-[(2R)-2-hydroxy-2-(4-methyl-1-oxo-1,3-dihydro-2-benzofuran-5-yl)ethyl]-9-pyridin-2-yl-9-aza-3-azoniaspiro[5.5]undecane

To a solution of (R)-5-(1-hydroxy-2-(3,9-diazaspiro[5.5]undecan-3-yl)ethyl)-4-methylisobenzofuran-1(3H)-one (INTERMEDIATE 81, 25 mg, 0.073 mmol) and 2-fluoropyridine (14 mg, 0.146 mmol) in anhydrous NMP (1 mL) was added NaHCO₃ (26 mg, 0.363 mmol). The resulting suspension was heated to 120° C. and shaked for 8 h. The reaction was cooled to ambient temperature and filtered. The crude product was purified by using reversed-phase HPLC (acetonitrile with 0.1% TFA: water with 0.1% TFA from 10% to 60%) to give the product as a yellow syrup. LC-MS (IE, m/z): 422 [M+1]⁺.

### Example 113

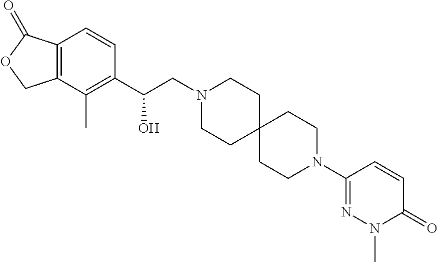 [SMILES: Cc1c([C@@H](O)CN2CCC3(CC2)CCN(c2ccc(=O)n(C)n2)CC3)ccc2c1COC2=O]

### (R)-6-(9-(2-hydroxy-2-(4-methyl-1-oxo-1,3-dihydroisobenzofuran-5-yl)ethyl)-3,9-diazaspiro[5.5]undecan-3-yl)-2-methylpyridazin-3(2H)-one

In 2 dram vial (8 mL) were added (R)-5-(1-hydroxy-2-(3,9-diazaspiro[5.5]undecan-3-yl)ethyl)-4-methylisobenzofuran-1(3H)-one dihydrochloride (INTERMEDIATE 81 45, 25 mg, 0.060 mmol), cesium carbonate (58.5 mg, 0.180 mmol), 6-bromo-2-methylpyridazin-3(2H)-one (22.64 mg, 0.120 mmol), 4,5-bis(diphenylphosphino)-9,9-dimethylxanthene (6.93 mg, 0.012 mmol) and tris(dibenzylideneacetone)dipalladium(0) (2.74 mg, 2.99 μmol) in THF (2 ml). The vial was degassed with N₂ and than stirred at 95° C. over night. The reaction mixture was filtered and purified with prep. LC/MS to give (R)-6-(9-(2-hydroxy-2-(4-methyl-1-oxo-1,3-dihydroisobenzofuran-5-yl)ethyl)-3,9-diazaspiro[5.5]undecan-3-yl)-2-methylpyridazin-3(2H)-one. LC-MS (IE, m/z): 453 [M+H]⁺.

The compounds in Table 10 were prepared in an analogous fashion to EXAMPLE 111-113 starting from (R)-5-(1-hydroxy-2-(3,9-diazaspiro[5.5]undecan-3-yl)ethyl)-4-methylisobenzofuran-1(3H)-one (INTERMEDIATE 81) or (R)-1-(6-(1H-tetrazol-1-yl)pyridin-3-yl)-2-(3,9-diazaspiro[5.5]undecan-3-yl)ethanol (INTERMEDIATE 82) and other commercially building intermediates.

TABLE 10

| | | | LC/MS |
|---|---|---|---|
| EX. | NAME | STRUCTURE | [M + 1]⁺ |
| 114 | (R)-5-(1-hydroxy-2-(9-(4- methoxypyrimidin-2-yl)- 3,9- diazaspiro[5.5]undecan-3- yl)ethyl)-4- methylisobenzofuran- 1(3H)-one | 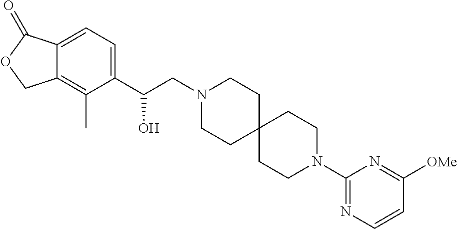 | 453 |
| 115 | (R)-5-(1-hydroxy-2-(9-(5- (trifluoromethyl)pyridin-2- yl)-3,9- diazaspiro[5.5]undecan-3- yl)ethyl)-4- methylisobenzofuran- 1(3H)-one | 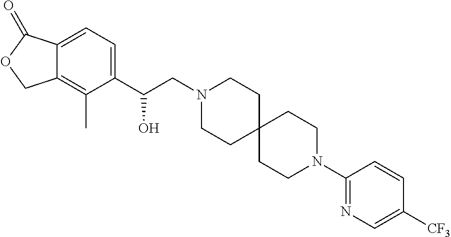 | 490 |
| 116 | (R)-5-(1-hydroxy-2-(9- (pyrazin-2-yl)-3,9- diazaspiro[5.5]undecan-3- yl)ethyl)-4- methylisobenzofuran- 1(3H)-one | 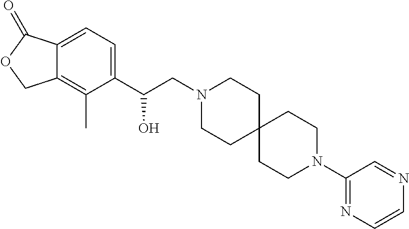 | 423 |
| 117 | (R)-5-(2-(9-(6- fluoropyrimidin-4-yl)-3,9- diazaspiro[5.5]undecan-3- yl)-1-hydroxyethyl)-4- methylisobenzofuran- 1(3H)-one | 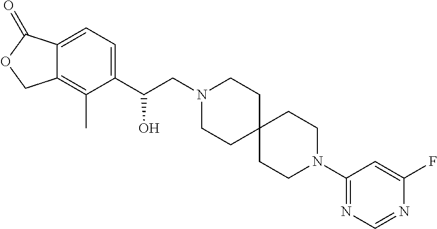 | 441 |
| 118 | (R)-5-(1-hydroxy-2-(9-(5- methoxypyrimidin-2-yl)- 3,9- diazaspiro[5.5]undecan-3- yl)ethyl)-4- methylisobenzofuran- 1(3H)-one | 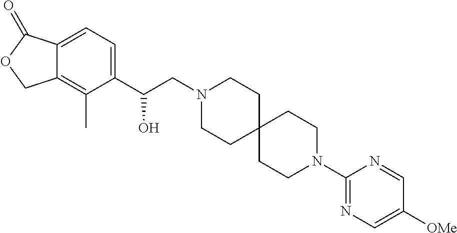 | 453 |
| 119 | (R)-5-(2-(9-(3-fluoro-5- (trifluoromethyl)pyridin-2- yl)-3,9- diazaspiro[5.5]undecan-3- yl)-1-hydroxyethyl)-4- methylisobenzofuran- 1(3H)-one | 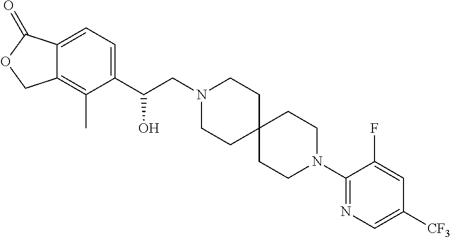 | 508 |
| 120 | (R)-5-(9-(2-hydroxy-2-(4- methyl-1-oxo-1,3- dihydroisobenzofuran-5- yl)ethyl)-3,9- diazaspiro[5.5]undecan-3- yl)pyrazine-2-carboxylic acid | 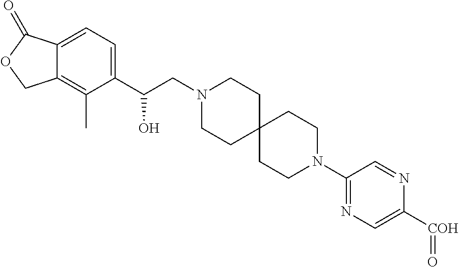 | 467 |
| 121 | (R)-6-(9-(2-hydroxy-2-(4- methyl-1-oxo-1,3- dihydroisobenzofuran-5- yl)ethyl)-3,9- diazaspiro[5.5]undecan-3- yl)pyrazine-2-carboxylic acid | 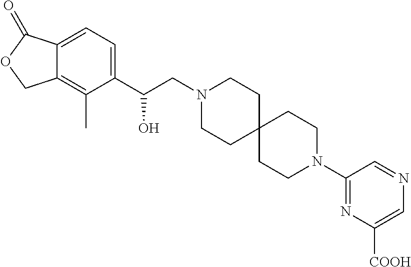 | 467 |
| 122 | (R)-5-(2-(9-(3- aminopyrazin-2-yl)-3,9- diazaspiro[5.5]undecan-3- yl)-1-hydroxyethyl)-4- methylisobenzofuran- 1(3H)-one | 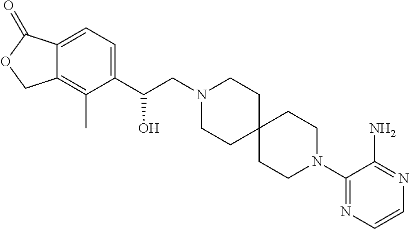 | 438 |
| 123 | (R)-5-(2-(9-(6- chloropyridazin-3-yl)-3,9- diazaspiro[5.5]undecan-3- yl)-1-hydroxyethyl)-4- methylisobenzofuran- 1(3H)-one | 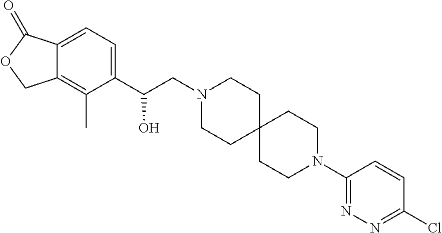 | 438 |
| 124 | (R)-5-(1-hydroxy-2-(9-(6- methylpyridazin-3-yl)-3,9- diazaspiro[5.5]undecan-3- yl)ethyl)-4- methylisobenzofuran- 1(3H)-one | 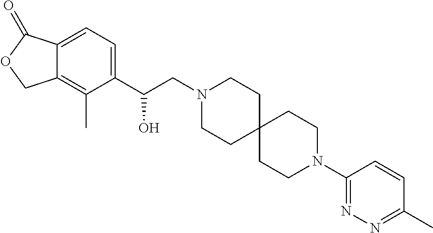 | 457 |
| 125 | (R)-5-(1-hydroxy-2-(9-(5- (trifluoromethyl)pyrazin-2- yl)-3,9- diazaspiro[5.5]undecan-3- yl)ethyl)-4- methylisobenzofuran- 1(3H)-one | 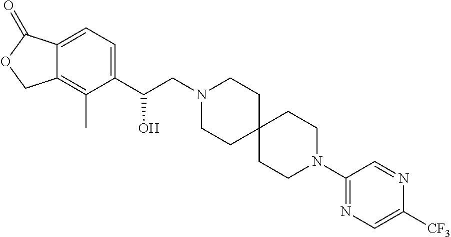 | 491 |
| 126 | (R)-5-(2-(9-(6- chloropyrazin-2-yl)-3,9- diazaspiro[5.5]undecan-3- yl)-1-hydroxyethyl)-4- methylisobenzofuran- 1(3H)-one | 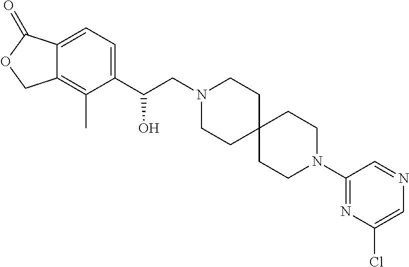 | 456 |
| 127 | (R)-5-(2-(9-(5- bromopyrazin-2-yl)-3,9- diazaspiro[5.5]undecan-3- yl)-1-hydroxyethyl)-4- methylisobenzofuran- 1(3H)-one | 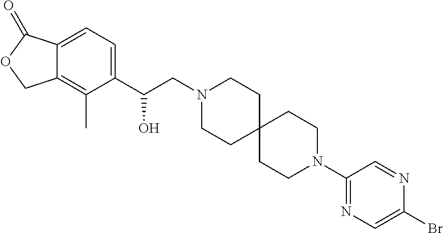 | 500 |
| 128 | (R)-5-(1-hydroxy-2-(9-(3- methylpyrazin-2-yl)-3,9- diazaspiro[5.5]undecan-3- yl)ethyl)-4- methylisobenzofuran- 1(3H)-one | 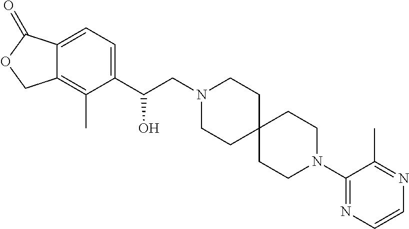 | 436 |
| 129 | (R)-5-(2-(9-(6- aminopyrazin-2-yl)-3,9- diazaspiro[5.5]undecan-3- yl)-1-hydroxyethyl)-4- methylisobenzofuran- 1(3H)-one | 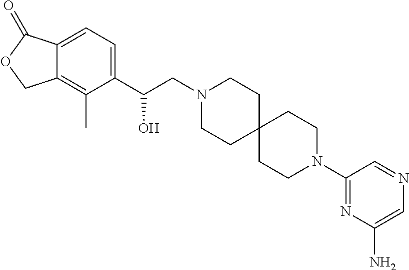 | 438 |
| 130 | (R)-5-(2-(9-(6-chloro-5- methylpyridazin-3-yl)-3,9- diazaspiro[5.5]undecan-3- yl)-1-hydroxyethyl)-4- methylisobenzofuran- 1(3H)-one | 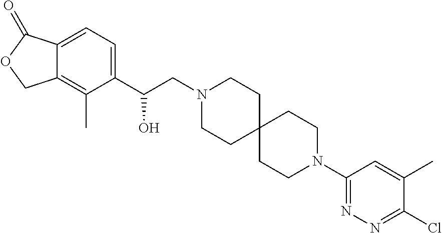 | 471 |
| 131 | (R)-5-(2-(9-(5- chloropyridazin-3-yl)-3,9- diazaspiro[5.5]undecan-3- yl)-1-hydroxyethyl)-4- methylisobenzofuran- 1(3H)-one | 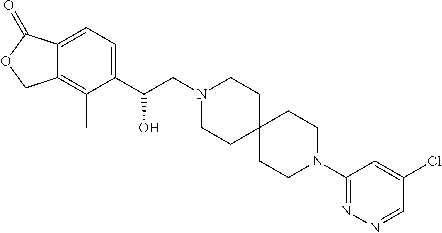 | 457 |
| 132 | (R)-5-(2-(9-(5- chloropyrazin-2-yl)-3,9- diazaspiro[5.5]undecan-3- yl)-1-hydroxyethyl)-4- methylisobenzofuran- 1(3H)-one | 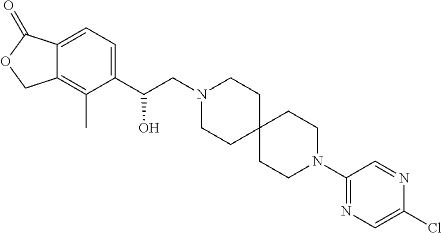 | 457 |
| 133 | (R)-5-(2-(9-(3- chloropyrazin-2-yl)-3,9- diazaspiro[5.5]undecan-3- yl)-1-hydroxyethyl)-4- methylisobenzofuran- 1(3H)-one | 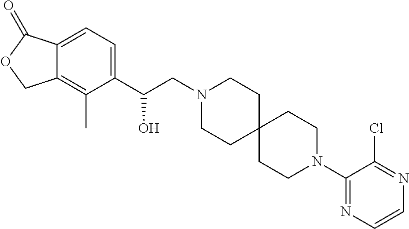 | 457 |
| 134 | (R)-1-(6-(1H-tetrazol-1- yl)pyridin-3-yl)-2-(9-(6- chloropyrazin-2-yl)-3,9- diazaspiro[5.5]undecan-3- yl)ethanol | 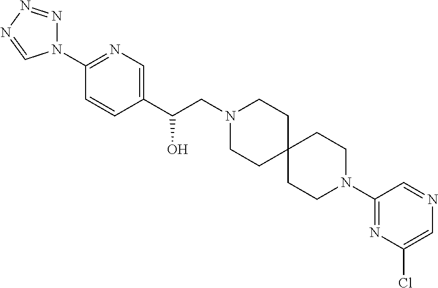 | 456 |
| 135 | (R)-1-(6-(1H-tetrazol-1- yl)pyridin-3-yl)-2-(9-(5- chloropyrazin-2-yl)-3,9- diazaspiro[5.5]undecan-3- yl)ethanol | 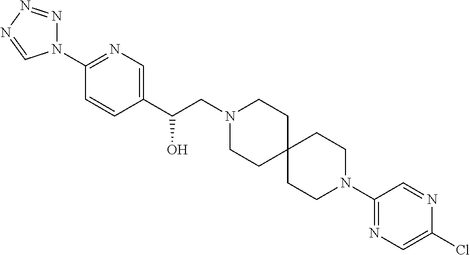 | 456 |
| 136 | (R)-5-(1-hydroxy-2-(9- (pyrimidin-2-yl)-3,9- diazaspiro[5.5]undecan-3- yl)ethyl)-4- methylisobenzofuran- 1(3H)-one | 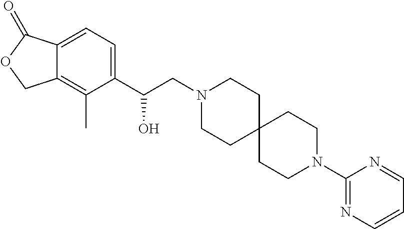 | 423 |
| 137 | (R)-5-(1-hydroxy-2-(9- (thiazol-2-yl)-3,9- diazaspiro[5.5]undecan-3- yl)ethyl)-4- methylisobenzofuran- 1(3H)-one | 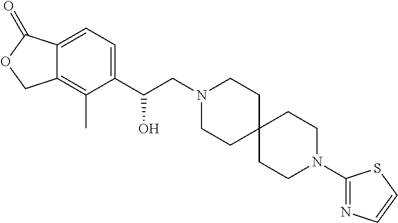 | 428 |
| 138 | (R)-1-(6-(1H-tetrazol-1- yl)pyridin-3-yl)-2-(9-(5- chloropyridazin-3-yl)-3,9- diazaspiro[5.5]undecan-3- yl)ethanol | 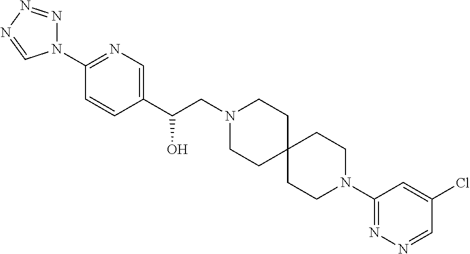 | 456 |
| 139 | (R)-5-(1-hydroxy-2-(9- (pyridazin-3-yl)-3,9- diazaspiro[5.5]undecan-3- yl)ethyl)-4- methylisobenzofuran- 1(3H)-one | 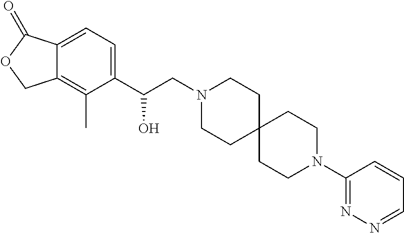 | 423 |
| 140 | (R)-methyl 2-(9-(2- hydroxy-2-(4-methyl-1- oxo-1,3- dihydroisobenzofuran-5- yl)ethyl)-3,9- diazaspiro[5.5]undecan-3- yl)thiazole-4-carboxylate | 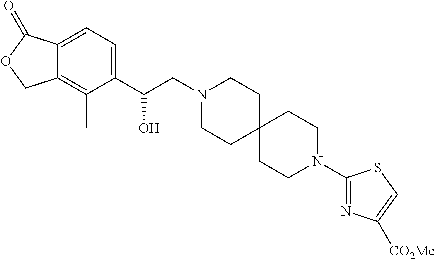 | 486 |
| 141 | (R)-5-(1-hydroxy-2-(9-(5- methoxypyrazin-2-yl)-3,9- diazaspiro[5.5]undecan-3- yl)ethyl)-4- methylisobenzofuran- 1(3H)-one | 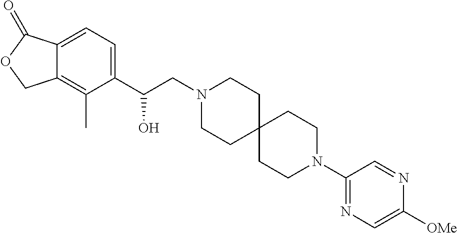 | 453 |
| 142 | (R)-5-(1-hydroxy-2-(9- (pyridazin-4-yl)-3,9- diazaspiro[5.5]undecan-3- yl)ethyl)-4- methylisobenzofuran- 1(3H)-one | 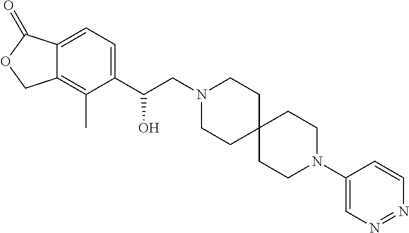 | 423 |

The following Thallium Flux Assay was performed on each of the final product compounds in the Examples.

### Thallium Flux Assay

### Cell Culture Conditions—

HEK293 cells stably expressing hROMK (hKᵢᵣ1.1) were grown at 37° C. in a 10% CO₂ humidified incubator in complete growth media: Dulbecco's Modified Eagle Medium supplemented with non-essential amino acids, Penicillin/Streptomycin/Glutamine, G418 and FBS. At >80% confluency, the media was aspirated from the flask and rinsed with 10 mL calcium/magnesium-free PBS. 5 mL of 1× trypsin (prepared in Ca/Mg Free PBS) was added to T-225 flask and flask was returned to 37° C./CO₂ incubator for 2-3 minutes. To dislodge the cell, the side of the flask was gently banged with one's hand. The cells completely titrated and then the cells were transferred to 25 mL complete media, centrifuged at 1,500 rpm for 6 min followed by resuspension in complete growth media and determine cell concentration. For typical re-seeding, 4E6 cells/T-225 flask will attain >80% confluency in 4 days. Under ideal growth conditions and appropriate tissue culture practices, this cell line is stable for 40-45 passages.

### FluxOR™ Kit Components (Invitrogen F10017)

FluxOR™ Reagent (Component A)
FluxOR™ Assay Buffer (Component B)—10× Concentrate
PowerLoad™ Concentrate (Component C)—100× Concentrate
Probenecid (Component D)—Lyophilized sample is kept at −20° C. Water soluble, 100× after solubilization in 1 mL water. Store at 4° C.
FluxOR™ Chloride-free Buffer (Component E)—5× Concentrate
Potassium sulfate (K₂SO₄) Concentrate (Component F)—125 mM in water. Store at 4° C.
Thallium sulfate (Tl₂SO₄) Concentrate (Component G)—50 mM in water. Store at 4° C.
DMSO (dimethyl sulfoxide, Component H)—1 mL (100%)

### Reagent Preparation: FluxOR Working Solutions

1000×FluxOR™ Reagent: Reconstitute a vial of component A in 100 μl DMSO; Mix well; Store 10 μl aliquots at −20° C.
1×FluxOR™ Assay Buffer: Dilute Component B 10-fold with water; Adjust pH to 7.4 with Hepes/NaOH; Filter and store at 4° C.
Probenecid/Assay Buffer: 100 mL of 1×FluxOR™ Assay Buffer; 1 mL of reconstituted component D; Store at 4° C.
Loading Buffer (per microplate): 10 μl 1000×FluxOR™ Reagent; 100 μl component C; 10 mL Probenecid/Assay Buffer
Compound Buffer (per microplate): 20 mL Probenecid/Assay Buffer; 0.3 mM ouabain (10 mM ouabain in water can be stored in amber bottle/aluminum foil at room temperature); Test compound
1×FluxOR™ Chloride-Free Buffer: Prepare 1× working solution in water. Can be stored at room temperature
Stimulant Buffer (prepared at 5× final concentration in 1×FluxOR™ Chloride-Free Buffer): 7.5 mM thallium sulfate and 0.75 mM potassium sulfate (to give a final assay concentration of 3 mM Thallium/0.3 mM potassium). Store at 4° C. when not in use. If kept sterile, this solution is good for months.

### Assay Protocol—

The ROMK channel functional thallium flux assay was performed in 384 wells, using the FLIPR-Tetra instrument. HEK-hKir1.1 cells were seeded in Poly-D-Lysine microplates and kept in a 37° C.-10% CO₂ incubator overnight. On the day of the experiment, the growth media was replaced with the FluxOR™ reagent loading buffer and incubated, protected from light, at ambient temperature (23-25° C.) for 90 min. The loading buffer was replaced with assay buffer±test compound followed by 30 min incubation at ambient temperature, where the thallium/potassium stimulant was added to the microplate.

### Step Protocol

1. Seed HEK-hKir1.1 cells (50 μl at 20,000 cells/well) in 384-well PDL coated Microplates
2. Allow cells to adhere overnight in humidified 37° C./10% CO₂ incubator
3. Completely remove cell growth media from microplate and replace with 25 μl loading buffer
4. Incubate Microplate at room temperature, protected form light, for 90 min
5. Remove loading buffer and replace with 25 μl 1× Assay Buffer±test compound.
6. Incubate microplate at room temperature, protected from light, for 30 min
7. At FLIPR-Tetra 384: Add stimulant (thallium/potassium) solution to microplate and monitor fluorescence. Excitation=400 nm, Emission=460 & 580 nm. Collect data for ˜10 min.

### Data Calculation—

The fluorescence intensity of wells containing 3 μM of a standard control ROMK inhibitor of the present invention was used to define the ROMK-sensitive component of thallium flux. Fluorescence in the presence of test compounds was normalized to control values to provide % fluorescence change. IC₅₀ values represent the concentration of compound that inhibited 50% of the ROMK thallium flux signal.

### Assay Standard—

Normally, a control compound is included to support that the assay is giving consistent results compared to previous measurements, although the control is not required to obtain the results for the test compounds. The control can be any compound of Formula I of the present invention, preferably with an IC₅₀ potency of less than 1 μM in this assay. Alternatively, the control could be another compound (outside the scope of Formula I) that has an IC₅₀ potency in this assay of less than 1 μM.

Data collected for compounds in the Examples of the present invention using the Thallium Flux Assay are shown in Table 11 below. All of the tested final product compounds in the Examples (diastereomeric mixtures and individual diastereomers) had IC₅₀ potencies less than 1 μM the Thallium Flux Assay.

TABLE 11

| Example | ROMK TI IC50 |
|---|---|
| 1 | 0.2508 |
| 2 | 0.9345 |
| 3 | 0.1004 |
| 4 | 0.04513 |
| 5 | 0.06478 |
| 6 | 0.4757 |
| 7 | 0.1491 |
| 8 | 0.5265 |
| 9 | 0.1735 |
| 10 | 0.7252 |
| 11 | 0.06758 |
| 12 | 0.9621 |
| 13 | 0.3364 |
| 14 | 0.4195 |
| 15 | 0.2839 |
| 16 | 0.4175 |
| 17 | 0.08834 |
| 18 | 0.3765 |
| 19 | 0.5398 |
| 20 | 0.05124 |
| 21 | 0.5684 |
| 22 | 0.4205 |
| 23 | 0.4066 |
| 24 | 0.03017 |
| 25 | 0.4368 |
| 26 | 0.2377 |
| 27 | 0.02502 |
| 28 | 0.1228 |
| 29 | 0.1446 |
| 30 | 0.0951 |
| 31 | 0.1534 |
| 32 | 0.4653 |
| 33 | 0.06308 |
| 34 | 0.5052 |
| 35 | 0.3788 |
| 36 | 0.3932 |
| 37 | 0.6763 |
| 38 | 0.08246 |
| 39 | 0.03878 |
| 40 | 0.1423 |
| 41 | 0.1926 |
| 42 | 0.8418 |
| 43 | 0.5207 |
| 44 | 0.06656 |
| 45 | 0.354 |
| 46 | 0.4854 |
| 47 | 0.05853 |
| 48 | 0.2857 |
| 49 | 0.1764 |
| 50 | 0.48 |
| 51 | 0.07704 |
| 52 | 0.4272 |
| 53 | 0.04063 |
| 54 | 0.2935 |
| 55 | 0.1334 |
| 56 | 0.0351 |
| 57 | 0.2523 |
| 58 | 0.036 |
| 59 | 0.462 |
| 60 | 0.005203 |
| 61 | 0.5466 |
| 62 | 0.34 |
| 63 | 0.1301 |
| 64 | 0.2415 |
| 65 | 0.0635 |
| 66 | 0.01752 |
| 67 | 0.4343 |
| 68 | 0.221 |
| 69 | 0.6726 |
| 70 | 0.6946 |
| 71 | 0.03714 |
| 72 | 0.01996 |
| 73 | 0.4175 |
| 74 | 0.05573 |
| 75 | 0.04664 |
| 76 | 0.3199 |
| 77 | 0.6966 |
| 78 | 0.02341 |
| 79 | 0.05666 |
| 80 | 0.0143 |
| 81 | 0.1749 |
| 82 | 0.4241 |
| 83 | 0.8317 |
| 84 | 0.1491 |
| 85 | 0.801 |
| 86 | 0.5607 |
| 87 | 0.6428 |
| 88 | 0.1295 |
| 89 | 0.7874 |
| 90 | 0.1196 |
| 91 | 0.7748 |
| 92 | 0.1099 |
| 93 | 0.2586 |
| 94 | 0.2721 |
| 95 | 0.4877 |
| 96 | 0.7023 |
| 97 | 0.3778 |
| 98 | 0.05467 |
| 99 | 0.2423 |
| 100 | 0.7692 |
| 101 | 0.2026 |
| 102 | 0.3494 |
| 103 | 0.06139 |
| 104 | 0.06549 |
| 105 | 0.05418 |
| 106 | 0.3608 |
| 107 | 0.04197 |
| 108 | 0.056 |
| 109 | 0.01998 |
| 110 | 0.279 |
| 111 | 0.1846 |
| 112 | 0.2816 |
| 113 | 0.616 |
| 114 | 0.3838 |
| 115 | 0.2251 |
| 116 | 0.006279 |
| 117 | 0.4033 |
| 118 | 0.1253 |
| 119 | 0.9674 |
| 120 | 0.2361 |
| 121 | 0.3139 |
| 122 | 0.1603 |
| 123 | 0.00845 |
| 124 | 0.0371 |
| 125 | 0.4917 |
| 126 | 0.2815 |
| 127 | 0.02987 |
| 128 | 0.5812 |
| 129 | 0.06828 |
| 130 | 0.553 |
| 131 | 0.02376 |
| 132 | 0.02277 |
| 133 | 0.2738 |
| 134 | 0.7468 |
| 135 | 0.8746 |
| 136 | 0.3304 |
| 137 | 0.2828 |
| 138 | 0.387 |
| 139 | 0.2511 |
| 140 | 0.364 |
| 141 | 0.3498 |
| 142 | 0.1703 |

While the invention has been described with reference to certain particular embodiments thereof, numerous alternative embodiments will be apparent to those skilled in the art from the teachings described herein. The scope of the claims should not be limited by the preferred embodiments set forth in the examples, but should be given the broadest interpretation consistent with the description as a whole. Recitation or depiction of a specific compound in the claims (i.e., a species) without a specific stereoconfiguration designation, or with such a designation for less than all chiral centers, is intended to encompass the racemate, racemic mixtures, each individual enantiomer, a diastereoisomeric mixture and each individual diastereomer of the compound where such forms are possible due to the presence of one or more asymmetric centers. All patents, patent applications and publications cited herein are incorporated by reference in their entirety.

1. A compound of the formula 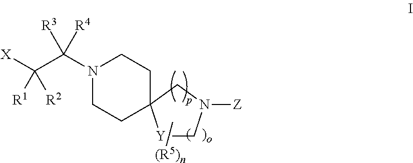
or a pharmaceutically acceptable salt thereof,
wherein:
X is 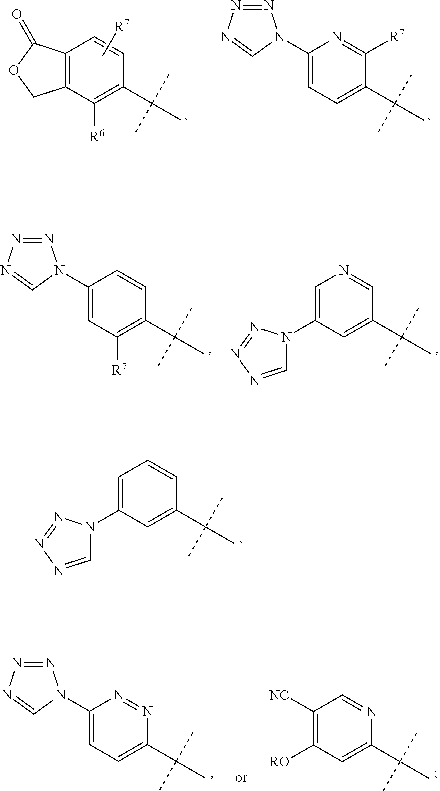
Y is —O—, —NH— or a bond;
Z is 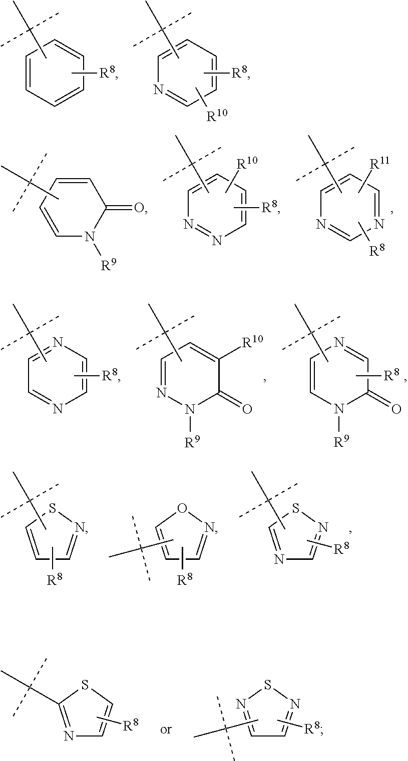
R is independently H, alkyl or haloalkyl;
R¹ is H, D or —OH;
R² is H or D;
R³ is H, D or alkyl;
R⁴ is H or D;
R⁵ is independently oxo or alkyl optionally substituted by 1-5 fluorine atoms;
R⁶ is H or alkyl;
R⁷ is H or alkyl;
R⁸ is H; halo; alkyl optionally substituted by —OR, —C(O)OR¹², —OC(O)—R¹², or 1-5 halogen atoms; —OR; phenyl; —C(O)OR¹³; —N(R¹⁴)(R¹⁵); furanyl; or —OCD₃;
R⁹ is H, alkyl optionally substituted by 1-5 halogen atoms, or cycloalkyl;
R¹⁰ is H, halo, or alkyl optionally substituted by 1-5 halogen atoms;
R¹¹ is H, alkyl optionally substituted by 1-5 halogen atoms, or —OR;
R¹² is H or alkyl;
R¹³ is H or alkyl;
R¹⁴ is H or alkyl;
R¹⁵ is H or alkyl;
n is 0, 1 or 2;
o is 1, 2 or 3; and
p is 1 or 2.

2. A compound as defined in claim 1, which has the formula 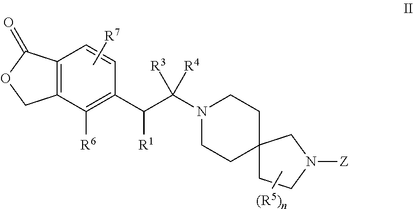
or a pharmaceutically acceptable salt thereof.

3. A compound as defined in claim 1, which has the formula 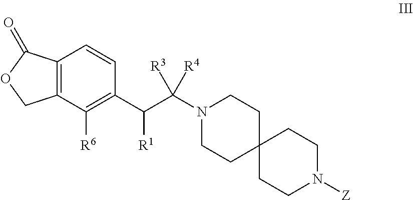
or a pharmaceutically acceptable salt thereof
wherein:
Z is

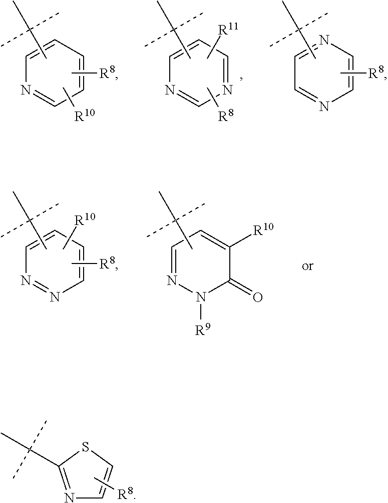

4. A compound as defined in claim 2, which one of the following formulae: 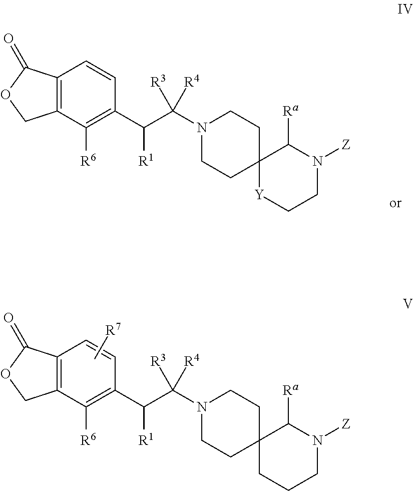
or a pharmaceutically acceptable salt thereof
wherein:
R^{a} is H or oxo;
Y is —O— or —NH—;
Z is

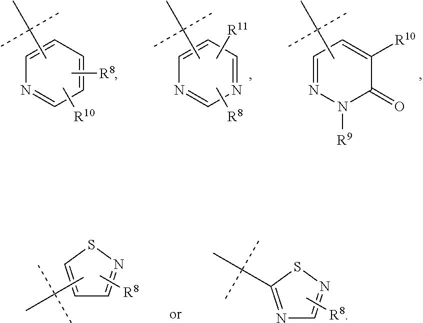

5. A compound as defined in claim 1, which has the formula 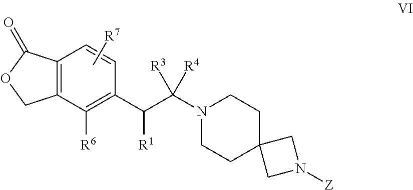
or a pharmaceutically acceptable salt thereof,
wherein:
Z is

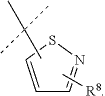

6. A compound as defined in claim 1, which has one of the following formulae: 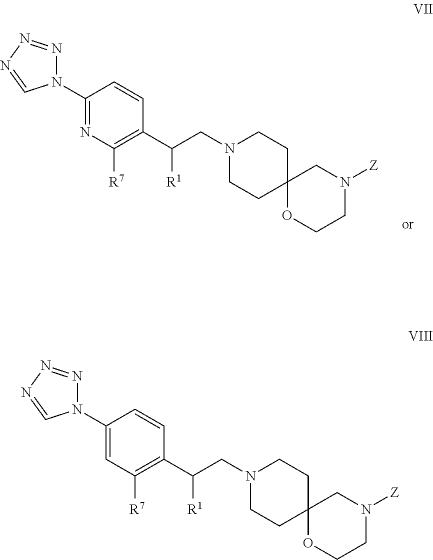
or a pharmaceutically acceptable salt thereof
wherein:
Z is

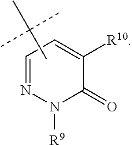

7. A compound as defined in claim 1, which has the formula 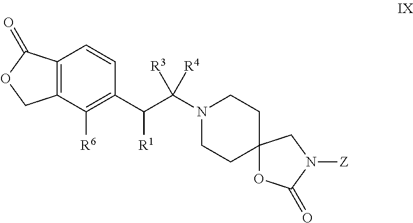
or a pharmaceutically acceptable salt thereof
wherein:
Z is

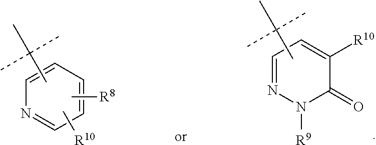

8. A compound as defined in claim 1 which has the formula 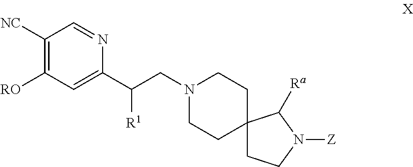
or a pharmaceutically acceptable salt thereof,
wherein:
R^{a} is H or oxo; and
Z is

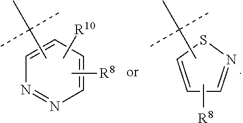

9. A compound as defined in claim 1 which has the formula 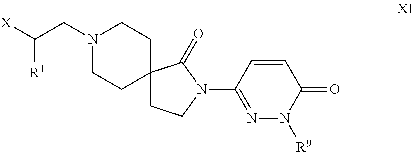
or a pharmaceutically acceptable salt thereof
wherein:
X is

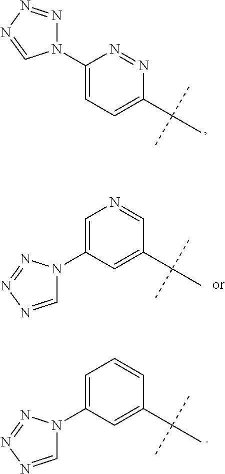

10. A compound as defined in claim 1 which has the formula: 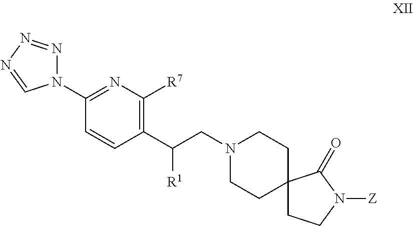
or a pharmaceutically acceptable salt thereof,
wherein:
Z is

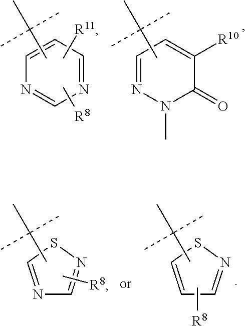

11. A compound as defined in claim 1, which has the formula 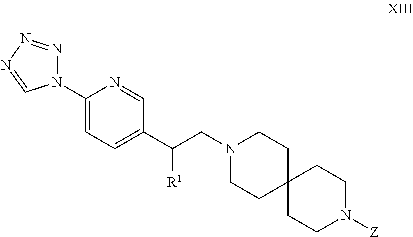
or a pharmaceutically acceptable salt thereof
wherein:
Z is 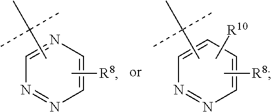
and
R¹ is H or —OH.

12. A compound as defined in claim 2 or a pharmaceutically acceptable salt thereof, which has the formula: 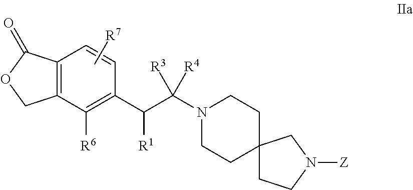
wherein:
Z is

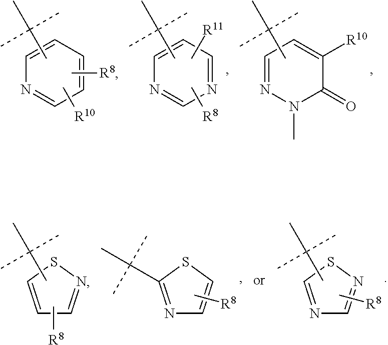

13. A compound as defined in claim 2 or a pharmaceutically acceptable salt thereof, which has the formula 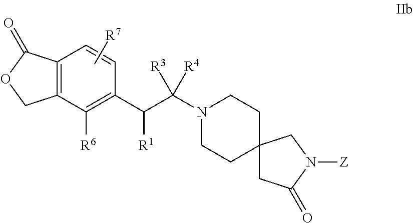
wherein
Z is

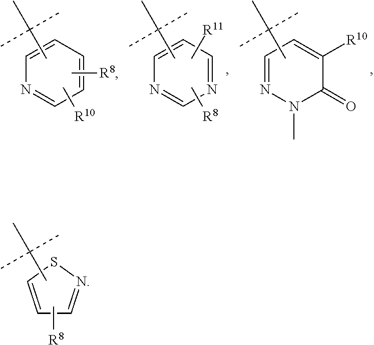

14. A compound as defined in claim 2 or a pharmaceutically acceptable salt thereof, which has the formula: 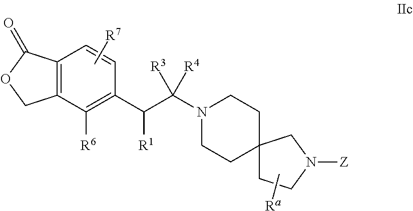
wherein:
R^{a} is H or alkyl; and
Z is

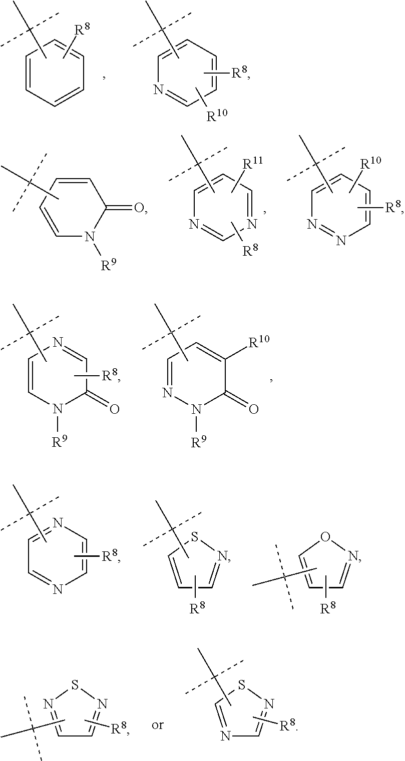

15. A compound of claim 1 which is:
(R)-8-(2-hydroxy-2-(4-methyl-1-oxo-1,3-dihydroisobenzofuran-5-yl)ethyl)-2-(isothiazol-5-yl)-2,8-diazaspiro[4.5]decan-1-one;
(R)-8-(2-hydroxy-2-(4-methyl-1-oxo-1,3-dihydroisobenzofuran-5-yl)ethyl)-2-(pyridin-4-yl)-2, 8-diazaspiro[4.5]decan-1-one;
(R)-8-(2-hydroxy-2-(4-methyl-1-oxo-1,3-dihydroisobenzofuran-5-yl)ethyl)-2-(3-methylisothiazol-5-yl)-2, 8-diazaspiro[4.5]decan-1-one
(R)-8-(2-hydroxy-2-(4-methyl-1-oxo-1,3-dihydroisobenzofuran-5-yl)ethyl)-2-(3-methyl-1,2,4-thiadiazol-5-yl)-2,8-diazaspiro[4.5]decan-1-one
R)-8-(2-hydroxy-2-(4-methyl-1-oxo-1,3-dihydroisobenzofuran-5-yl)ethyl)-2-(isothiazol-4-yl)-2,8-diazaspiro[4.5]decan-1-one;
(R)-8-(2-hydroxy-2-(4-methyl-1-oxo-1,3-dihydroisobenzofuran-5-yl)ethyl)-2-(2-methoxypyridin-4-yl)-2, 8-diazaspiro[4.5]decan-1-one;
(R)-8-(2-hydroxy-2-(4-methyl-1-oxo-1,3-dihydroisobenzofuran-5-yl)ethyl)-2-(3-methoxy-1,2,4-thiadiazol-5-yl)-2,8-diazaspiro[4.5]decan-1-one;
(R)-8-(2-hydroxy-2-(4-methyl-1-oxo-1,3-dihydroisobenzofuran-5-yl)ethyl)-2-(pyridin-4-yl)-2, 8-diazaspiro[4.5]decan-3-one;
(R)-8-(2-hydroxy-2-(4-methyl-1-oxo-1,3-dihydroisobenzofuran-5-yl)ethyl)-2-(2-methoxypyrimidin-4-yl)-2, 8-diazaspiro[4.5]decan-1-one;
(R)-8-(2-hydroxy-2-(4-methyl-1-oxo-1,3-dihydroisobenzofuran-5-yl)ethyl)-2-(2-methoxypyrimidin-4-yl)-2,8-diazaspiro[4.5]decan-1-one (methoxy-d₃);
(R)-8-(2-hydroxy-2-(4-methyl-1-oxo-1,3-dihydroisobenzofuran-5-yl)ethyl)-2-(2-(methylamino)pyrimidin-4-yl)-2,8-diazaspiro[4.5]decan-1-one;
(R)-8-(2-hydroxy-2-(4-methyl-1-oxo-1,3-dihydroisobenzofuran-5-yl)ethyl)-2-(1-methyl-6-oxo-1,6-dihydropyridazin-3-yl)-2,8-diazaspiro[4.5]decan-1-one;
(R)-8-(2-hydroxy-2-(4-methyl-1-oxo-1,3-dihydroisobenzofuran-5-yl)ethyl-d₃)-2-(1-methyl-6-oxo-1,6-dihydropyridazin-3-yl)-2,8-diazaspiro[4.5]decan-1-one;
8-((1R,2S)-1-hydroxy-1-(4-methyl-1-oxo-1,3-dihydroisobenzofuran-5-yl)propan-2-yl)-2-(1-methyl-6-oxo-1,6-dihydropyridazin-3-yl)-2,8-diazaspiro[4.5]decan-1-one;
8-((1 S,2S)-1-hydroxy-1-(4-methyl-1-oxo-1,3-dihydroisobenzofuran-5-yl)propan-2-yl)-2-(1-methyl-6-oxo-1,6-dihydropyridazin-3-yl)-2,8-diazaspiro[4.5]decan-1-one; or
(R)-9-(2-hydroxy-2-(4-methyl-1-oxo-1,3-dihydroisobenzofuran-5-yl)ethyl)-4-(3-methoxy-1,2,4-thiadiazol-5-yl)-1-oxa-4,9-diazaspiro[5.5]undecan-5-one
or a pharmaceutically acceptable salt thereof.

16. A pharmaceutical composition comprising a therapeutically effective amount of a compound as defined in claim 1 or a pharmaceutically acceptable salt thereof and a pharmaceutically acceptable carrier.

17. The pharmaceutical composition as defined in claim 16, which further comprises a therapeutically effective amount of at least one additional therapeutic agent.

18. The pharmaceutical composition as defined in claim 17, wherein the additional therapeutic agent is losartan, valsartan, candesartan, olmesartan, telmesartan, eprosartan, irbesartan, amlodipine, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril, amiloride, spironolactone, epleranone or triamterene, or a pro-drug thereof, or a pharmaceutically acceptable salt of any of the foregoing.

19. A method for causing natriuresis or both, comprising administering a compound of claim 1 or a pharmaceutically acceptable salt thereof in a therapeutically effective amount to a patient in need thereof.

20. A method for the treatment of one or more disorders selected from hypertension, acute heart failure, chronic heart failure, pulmonary arterial hypertension, cardiovascular disease, diabetes, endothelial dysfunction, diastolic dysfunction, stable and unstable angina pectoris, thromboses, restenosis, myocardial infarction, stroke, cardiac insufficiency, pulmonary hypertonia, atherosclerosis, hepatic cirrhosis, ascitis, pre-eclampsia, cerebral edema, nephropathy, nephrotic syndrome, acute kidney insufficiency, chronic kidney disease, hypercalcemia, Dent's disease, Meniere's disease, or edematous states in a patient in need thereof comprising administering an effective amount of a compound as defined in claim 1 or a pharmaceutically acceptable salt thereof to said patient.

21. A method for inhibiting ROMK comprising administering in a patient in need thereof which comprises administering an effective amount of a compound of the formula 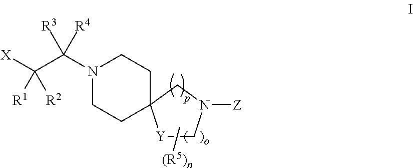
or a pharmaceutically acceptable salt thereof,
wherein:
X is 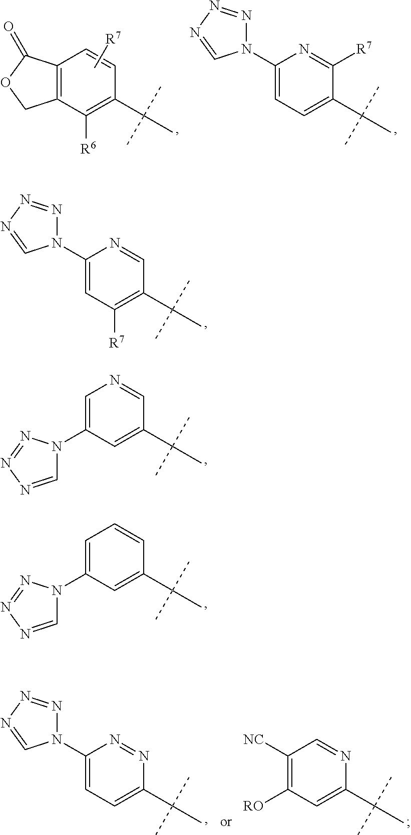
Y is —O—, —NH— or a bond;
Z is 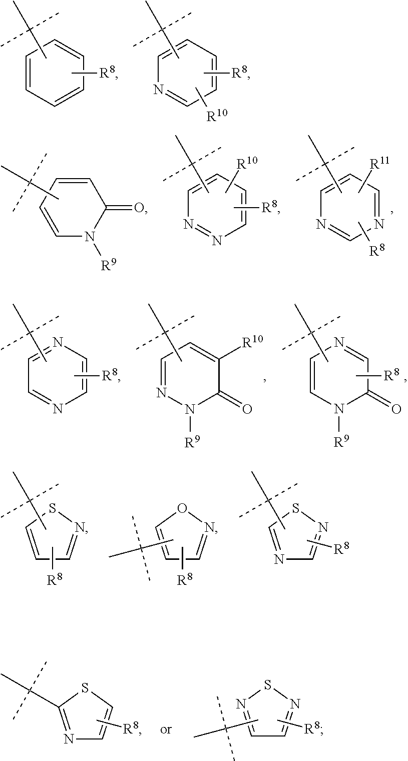
R is independently H, alkyl or haloalkyl;
R¹ is H, D or —OH;
R² is H or D;
R³ is H, D or alkyl;
R⁴ is H or D;
R⁵ is independently oxo or alkyl optionally substituted by 1-5 fluorine atoms;
R⁶ is H or alkyl;
R⁷ is H or alkyl;
R⁸ is H; halo; alkyl optionally substituted by —OR, —C(O)OR¹², —OC(O)—R¹², or 1-5 halogen atoms; —OR; phenyl; —C(O)OR¹³; —N(R¹⁴)(R¹⁵); furanyl; or —OCD₃;
R⁹ is H, alkyl optionally substituted by 1-5 halogen atoms, or cycloalkyl;
R¹⁰ is H, halo, or alkyl optionally substituted by 1-5 halogen atoms;
R¹¹ is H, alkyl optionally substituted by 1-5 halogen atoms, or —OR;
R¹² is H or alkyl;
R¹³ is H or alkyl;
R¹⁴ is H or alkyl;
R¹⁵ is H or alkyl;
n is 0, 1 or 2;
o is 1, 2 or 3; and
p is 1 or 2.
